(19) United States
(12) Patent Application Publication
(10) Pub. No.: US 2024/0343794 A1
(43) Pub. Date: Oct. 17, 2024

(54) COMPOSITIONS AND METHODS FOR THE TREATMENT OF INTESTINAL CANCER

(71) Applicant: United Kingdom Research and Innovation, Swindon (GB)

(72) Inventors: Andrew MCKENZIE, Cambridge (GB); Eric JOU, Cambridge (GB)

(21) Appl. No.: 18/037,063

(22) PCT Filed: Nov. 15, 2021
(86) PCT No.: PCT/EP2021/081750

§ 371 Date: May 15, 2023

(30) Foreign Application Priority Data
Nov. 16, 2020 (GB) 2018015.4

(51) Int. Cl. C07K 16/24; A61K 39/395; A61K 45/06; A61P 35/00

(52) U.S. Cl. CPC C07K 16/244; A61K 39/3955; A61K 45/06; A61P 35/00

(57) ABSTRACT

The present invention relates to the treatment of cancer of the intestine. The invention extends to inhibitors of the IL-25:IL-25R signalling pathway, which specifically bind to IL-25 or to IL-25R, for use in methods of treating, preventing, or amelio-rating cancer of the intestine.

### FIELD OF THE INVENTION

The present invention relates to the treatment of cancer of the intestine. The invention extends to inhibitors of the IL-25:IL-25R signalling pathway for use in methods of treating, preventing, or ameliorating cancer of the intestine.

### BACKGROUND

Intestinal cancer is a common cause of cancer-related death. For example, colorectal cancer (CRC) is the second most common cause of cancer-related death worldwide, and is expected to increase by a further 60% by 2030. The aim is to diagnose early and allow curative treatments, usually involving surgery, and in some cases in combination with adjuvant and neoadjuvant treatments. For patients intolerant to surgery, or with unresectable lesions, the goal is to maximally shrink the tumours and suppress further tumour growth. Unfortunately, current checkpoint inhibitor therapies are ineffectual in CRC. Approximately 80% of human CRC have mutations in the adenomatous polyposis coli (APC) tumour suppressor gene, and follow a well-defined pathway of loss of heterozygosity (LOH) of APC in intestinal epithelial cells, aberrant activation of Wnt signalling and polyp formation.

Despite the recent advances in immune checkpoint inhibitor therapy, showing efficacy in various different types of cancers, the response in CRC patients is largely limited to mismatch-repair-deficient (dMMR) CRC, comprising only a small proportion (15%) of total CRC cases. By contrast, the vast majority of CRC, following the conventional pathway initiated by mutations in the APC gene, are mismatch-repair-proficient (pMMR), and rarely respond to immune checkpoint inhibitors. Furthermore, in the past decade, efforts to develop novel immunotherapies for CRC, such as tumour vaccines and adoptive T cell transfer, have shown little efficacy. Recently, the phase III clinical trial IMblaze370 using the anti-PD-L1 checkpoint inhibitor atezolizumab to treat CRC patients (>98% had mismatch-repair-proficient CRC) failed to meet the primary end point.

Thus, there is a need for new drugs to treat intestinal cancer.

Interleukin-25 (IL-25) is a cytokine that is expressed by epithelial cells in the intestine, and studies have implied that IL-25 may play an inhibitory role in the growth and development of colonic tumours. Thelen et al. reported that acute blockade of IL-25 in a colitis associated colon cancer model leads to increased tumour burden (Thelen, T., Green, R. & Ziegler, S. Acute blockade of IL-25 in a colitis associated colon cancer model leads to increased tumor burden. Sci Rep 6, 25643 (2016)).

Benatar et al. reported that injection of recombinant IL-25 resulted in significant antitumour activity for melanoma, lung, breast, colon and pancreatic cancer in human tumour xenograft models (Benatar, T., Cao, M. Y., Lee, Y. et al. IL-17E, a proinflammatory cytokine, has antitumor efficacy against several tumor types in vivo. Cancer Immunol Immunother 59, 805-817 (2010)).

Thus, in light of these studies, it might be expected that agonists of the IL-25:IL-25R signalling pathway would be useful in the treatment of intestinal cancer.

Group 2 innate lymphoid cells (ILC2s) are a type of immune cell that can help to defend the host against intestinal parasitic helminth infection, and are associated with inappropriate allergic reactions. Moral et al. reported that ILC2s infiltrate pancreatic ductal adenocarcinomas (PDACs) to activate tissue-specific tumour immunity, and suggest that ILC2s are anti-cancer immune cells for PDAC (Moral, J. A., Leung, J.,

Rojas, L. A. et al. ILC2s amplify PD-1 blockade by activating tissue-specific cancer immunity. Nature 579, 130-135 (2020)).

Thus, in light of this study, it might be expected that activation of ILC2s would be useful in the treatment of cancer.

WO2017/194554 discloses experiments relating to the effects of IL-25 on triple-negative breast cancer (TNBC) cell lines. The authors suggest that IL-25 might contribute to TNBC tumour resistance to EGRF inhibitors.

### SUMMARY OF THE INVENTION

In an aspect of the invention, there is provided an inhibitor of the IL-25:IL-25R signalling pathway for use in a method of treating, preventing, or ameliorating cancer of the intestine, wherein the inhibitor specifically binds to IL-25 or to IL-25R.

The cancer may be colorectal cancer. The cancer may comprise a mutation in the adenomatous polyposis coli (APC) tumour suppressor gene. The cancer may comprise a tumour that has an increased frequency of group 2 innate lymphoid cells (ILC2s) compared to the ILC2 frequency in adjacent normal intestine

The inhibitor may specifically bind to IL-25. The inhibitor may bind to one or more amino acid sequences selected from the group consisting of amino acid residues 46-63 of SEQ ID NO: 48, amino acid residues 66-84 of SEQ ID NO: 48, and amino acid residues 129-135 of SEQ ID NO: 48.

The inhibitor may be or may comprise an anti-IL-25 antibody, or functional fragment or derivative thereof. Thus, there is provided an anti-IL-25 antibody, or functional fragment or derivative thereof for use in a method of treating, preventing, or ameliorating cancer of the intestine.

The anti-IL-25 antibody, or functional fragment or derivative thereof, may comprise:
an antibody VH domain, or functional fragment or derivative thereof, comprising:
   a VH CDR1 comprising the amino acid sequence shown in any one of SEQ ID NOs: 1, 2, 12, 13, 14, 15, 16, 17, 18, or 19;
   a VH CDR2 comprising the amino acid sequence shown in SEQ ID NO: 3; and
   a VH CDR3 comprising the amino acid sequence shown in any one of SEQ ID NOs: 4, 20, or 21; and
an antibody a VL domain, or functional fragment or derivative thereof, comprising:
   a VL CDR1 comprising the amino acid sequence shown in SEQ ID NO: 5 or SEQ ID NO: 6;
   a VL CDR2 comprising the amino acid sequence shown in SEQ ID NO: 7; and
   a VL CDR3 comprising the amino acid sequence shown in any one of SEQ ID NOs: 8, 9, 44, or 79.

The anti-IL-25 antibody, or functional fragment or derivative thereof, may comprise:
an antibody VH domain, or functional fragment or derivative thereof, comprising:
   a VH CDR1 comprising the amino acid sequence shown in SEQ ID NO: 12;
   a VH CDR2 comprising the amino acid sequence shown in SEQ ID NO: 3; and
   a VH CDR3 comprising the amino acid sequence shown in SEQ ID NO: 20; and
an antibody a VL domain, or functional fragment or derivative thereof, comprising:
   a VL CDR1 comprising the amino acid sequence shown in SEQ ID NO: 5 or SEQ ID NO: 6;
   a VL CDR2 comprising the amino acid sequence shown in SEQ ID NO: 7; and
   a VL CDR3 comprising the amino acid sequence shown in SEQ ID NO: 8 or SEQ ID NO: 9.

The anti-IL-25 antibody, or functional fragment or derivative thereof, may comprise:
an antibody VH domain, or functional fragment or derivative thereof, comprising:
   a VH CDR1 comprising the amino acid sequence shown in SEQ ID NO: 12;
   a VH CDR2 comprising the amino acid sequence shown in SEQ ID NO: 3; and
   a VH CDR3 comprising the amino acid sequence shown in SEQ ID NO: 20; and
an antibody a VL domain, or functional fragment or derivative thereof, comprising:
   a VL CDR1 comprising the amino acid sequence shown in SEQ ID NO: 6;
   a VL CDR2 comprising the amino acid sequence shown in SEQ ID NO: 7; and
   a VL CDR3 comprising the amino acid sequence shown in SEQ ID NO: 44 or SEQ ID NO: 79.

The anti-IL-25 antibody, or functional fragment or derivative thereof, may comprise:
an antibody VH domain comprising the amino acid sequence shown in any one of SEQ ID NOs: 10, 22, 24, 25, 26, 27, 28, 29, 30, 31, 32, 33, 34, 35, 36, 37, or 38, and
an antibody VL domain comprising the amino acid sequence shown in any one of SEQ ID NOS: 11, 41, or 47;
wherein the antibody VH domain and/or antibody VL domain may optionally comprise from 1 to 10, from 1 to 5, from 1 to 3, one amino acid substitution, or no amino acid substitutions, compared to the recited sequence, in residues that are not within a CDR.

The anti-IL-25 antibody, or functional fragment or derivative thereof, may comprise:
an antibody VH domain comprising the amino acid sequence shown in SEQ ID NO: 24, and
an antibody VL domain comprising the amino acid sequence shown in SEQ ID NO: 11 or SEQ ID NO: 41.

The anti-IL-25 antibody, or functional fragment or derivative thereof, may comprise:
an antibody VH domain comprising the amino acid sequence shown in SEQ ID NO: 25, and
an antibody VL domain comprising the amino acid sequence shown in SEQ ID NO: 41.

The anti-IL-25 antibody, or functional fragment or derivative thereof, may comprise:
an antibody VH domain comprising the amino acid sequence shown in SEQ ID NO: 26, and
an antibody VL domain comprising the amino acid sequence shown in SEQ ID NO: 41.

The anti-IL-25 antibody, or functional fragment or derivative thereof, may comprise:
an antibody VH domain comprising the amino acid sequence shown in SEQ ID NO: 25, and
an antibody VL domain comprising the amino acid sequence shown in SEQ ID NO: 46.

The inhibitor may specifically bind to IL-25R. The inhibitor may be or may comprise an anti-IL-25R antibody, or functional fragment or derivative thereof. Thus, there is provided an anti-IL-25R antibody, or functional fragment or derivative thereof for use in a method of treating, preventing, or ameliorating cancer of the intestine.

The anti-IL-25R antibody, or functional fragment or derivative thereof may comprise:
an antibody VH domain, or functional fragment or derivative thereof, comprising:
   a VH CDR1 comprising the amino acid sequence shown in SEQ ID NO: 49;
   a VH CDR2 comprising the amino acid sequence shown in any one of SEQ ID NOS: 50, 57, 58, 59, 60, 61, 62, 63, or 64; and
   a VH CDR3 comprising the amino acid sequence shown in SEQ ID NO: 51; and
an antibody a VL domain, or functional fragment or derivative thereof, comprising:
   a VL CDR1 comprising the amino acid sequence shown in SEQ ID NO: 52;
   a VL CDR2 comprising the amino acid sequence shown in SEQ ID NO: 53; and
   a VL CDR3 comprising the amino acid sequence shown in SEQ ID NO: 54 or SEQ ID NO: 65.

The anti-IL-25R antibody, or functional fragment or derivative thereof may comprise:
an antibody VH domain, or functional fragment or derivative thereof, comprising:
   a VH CDR1 comprising the amino acid sequence shown in SEQ ID NO: 49;
   a VH CDR2 comprising the amino acid sequence shown in SEQ ID NO: 50 or SEQ ID NO: 64; and
   a VH CDR3 comprising the amino acid sequence shown in SEQ ID NO: 51; and
an antibody a VL domain, or functional fragment or derivative thereof, comprising:
   a VL CDR1 comprising the amino acid sequence shown in SEQ ID NO: 52;
   a VL CDR2 comprising the amino acid sequence shown in SEQ ID NO: 53; and
   a VL CDR3 comprising the amino acid sequence shown in SEQ ID NO: 65.

The anti-IL-25R antibody, or functional fragment or derivative thereof may comprise:
an antibody VH domain comprising the amino acid sequence shown in any one of SEQ ID NOs: 66, 67, 68, 69, 70, 80, 81, 82, 83, 84, 85, or 86, and
an antibody VL domain comprising the amino acid sequence shown in any one of SEQ ID NOs: 56, 71, 72, 87, 88, 89, 90, or 91;
wherein the antibody VH domain and/or antibody VL domain may optionally comprise from 1 to 10, from 1 to 5, from 1 to 3, one amino acid substitution, or no amino acid substitutions, compared to the recited sequence, in residues that are not within a CDR.

The anti-IL-25R antibody, or functional fragment or derivative thereof may comprise:
an antibody VH domain comprising the amino acid sequence shown in any one of SEQ ID NOS: 55, 66, 67, 68, 69, or 70, and
an antibody VL domain comprising the amino acid sequence shown in any one of SEQ ID NOs: 56, 71, or 72;
wherein the antibody VH domain and/or antibody VL domain may optionally comprise from 1 to 10, from 1 to 5, from 1 to 3, one amino acid substitution, or no amino acid substitutions, compared to the recited sequence, in residues that are not within a CDR.

The anti-IL-25R antibody, or functional fragment or derivative thereof may comprise:
an antibody VH domain comprising the amino acid sequence shown in any one of SEQ ID NOs: 68, 69, or 70, and
an antibody VL domain comprising the amino acid sequence shown in SEQ ID NO: 72.

The anti-IL-25 antibodies or the anti-IL-25R antibodies disclosed herein may optionally comprise at least one CDR sequence that comprises one, two, or three amino acid substitutions compared to a CDR recited sequence recited herein. In other embodiments, the anti-IL-25 antibodies or the anti-IL-25R antibodies disclosed herein may optionally comprise at least two, three, four, five, or six CDR sequences that comprise one, two, or three amino acid substitutions compared to a CDR recited sequence recited herein

The inhibitor of the IL-25:IL-25R signalling pathway may be an agent that competes for binding with any one of the inhibitors disclosed herein.

The inhibitor of the IL-25:IL-25R signalling pathway may be an antibody mimetic, or a functional fragment or derivative thereof.

The methods of treating, preventing, or ameliorating cancer of the intestine disclosed herein, may further comprise the administration of an immune checkpoint inhibitor. The immune checkpoint inhibitor may be an inhibitor of the PD-1: PD-L1 pathway.

### DETAILED DESCRIPTION

The inventors provide data herein that demonstrate a role for IL-25-activated ILC2s in the intestine where they create a cancer-permissive microenvironment.

The inventors show that in a mouse model of spontaneous intestinal tumorigenesis exogenous IL-25 increases tumours, whilst ablation of IL-25-signalling reduces tumour burden and virtually doubles life-expectancy, while reducing IL-25-responsive tumour

ILC2s and shifting the balance towards cytotoxic CD8 T cell infiltration. Without being bound to a particular theory, the inventors provide evidence herein to show that blockade of IL-25:IL25R signalling has a therapeutic effect due to effects mediated by ILC2S.

Thus, in a first aspect of the invention, there is provided an inhibitor of the IL-25:IL-25R signalling pathway for use in a method of treating, preventing, or ameliorating cancer of the intestine, wherein the inhibitor specifically binds to IL-25 or to IL-25R.

Previous reports addressing the role of IL-25:IL-25R signalling in intestinal cancer have reached the opposite conclusions to the present inventors. As discussed in the background section, Thelen et al. and Benatar et al. suggest that IL-25 has an anti-tumour effect, and thus suggest that the blockade of IL-25 activity would not be a suitable therapeutic strategy for the treatment of colonic tumours. The inventors believe that previous studies may have failed to discover the present invention due to deficiencies of the previously examined models of intestinal cancer. For instance, several studies have shown that chemical-induced mouse models of colitis-associated cancer (CAC), such as those that rely on the induction of cancer by treatment with azoxymethane (AOM) and dextran sodium sulphate (DSS), do not accurately reflect the genomic alterations found in sporadic human CRC. Thelen et al. makes use of a model involving AOM and DSS. In addition, xenograft models featuring immunodeficient mice, such as those used in Benatar et al. may not accurately recreate the tumour microenvironment, particularly with respect to immune cell infiltration.

The present inventors make use of the Apc^{1322T/+} mouse model of intestinal cancer. Intestinal tumours in APC-mutant mouse models are driven by the loss of the APC tumour suppressor gene. Specifically, the Apc^{1322T/+} model very closely mimics the mutations found in human cancer (Jackstadt, R. & Sansom, O. J. Mouse models of intestinal cancer. J Pathol 238, 141-151, doi: 10.1002/path.4645 (2016)).

A previous report has suggested that ILC2s are anti-cancer immune cells for PDAC (Moral et al; discussed in the background section), and hence play the opposite role in PDAC. The inventors conclude that the roles of the epithelium-derived cytokines IL-25 and IL-33, and ILC2s in tumorigenesis are likely to be location-dependent and organ specific.

Thus, the present invention is particularly surprising in light of previous studies.

The inhibitor of the IL-25:IL-25R signalling pathway may be any which, upon administration or application to a patient, is capable of binding to IL-25 or IL-25R and blocking signalling resulting from the binding of IL-25 to its receptor. The inhibitor may inhibit the functional interaction of IL-25R and IL-25.

Whether an agent is capable of inhibiting the IL-25:IL-25R signalling pathway may be measured by any technique known in the art. For instance, an in vitro assay may be used to measure whether the inhibitor is able to reduce or abolish IL-13 release from non B/non T (NB/NT) cells isolated from the mesenteric lymph nodes of mice. In this assay cells are incubated with or without IL-25 and varying concentrations of inhibitor or control. The IL-25 may be recombinant IL-25 at a concentration of 10 ng/ml. As a readout, IL-13 levels may be measured post stimulation, for instance three days post stimulation. IL-13 levels may be measured by ELISA. In some embodiments, the agent is considered to be an inhibitor of the IL-25:IL-25R signalling pathway if it is capable of reducing IL-13 release to the same extent as an anti-IL-25 or anti-IL-25R antibody disclosed herein. In an embodiment, the agent is considered to be an inhibitor of the IL-25:IL-25R signalling pathway if it is capable of reducing IL-13 release to the same extent as the antibody 2C3 or the antibody D9.2 disclosed herein, or their humanised versions. The humanised versions may include RH2.5_R71V, RH2.5_S30T, RH2.5, or M6 (discussed herein). In some embodiments, an agent or an antibody is considered to be an inhibitor of the IL-25:IL-25R signalling pathway if it is capable of ablating IL-13 production when tested at a concentration of 4 μg/ml, 2 μg/ml, 0.5 μg/ml, 0.25 μg/ml, or 0.1 μg/ml in the in vitro assay. For potential inhibitors with a different molecular weight to antibodies, the concentrations may be adjusted accordingly.

In another embodiment, whether an agent is an inhibitor of the IL-25:IL-25R signalling pathway is measured by determining whether the agent is able to reduce the activation of STAT, e.g. determined by measuring p-STAT5 levels, in a IL-25R⁺ cell exposed to IL-25 (Wu et al.; J Immunol. 2015 May 1; 194 (9): 4528-4534; herein incorporated by reference).

In other embodiments, whether an agent is an inhibitor of the IL-25:IL-25R signalling pathway is measured by determining whether the agent is able to reduce the incidence of tumours in young-adult Apc^{1322T/+} mice treated with said inhibitor, but has no effect or a reduced effect in young-adult Apc^{1322T/+} mice that are deficient in IL-25R and/or IL-25. Treatment of a young-adult mouse may be treatment from three weeks of age for a further two, three, four, five, six, seven, or more weeks. In a particular embodiment, the treatment is for seven weeks. The treatment may be one, twice, thrice, four times, five times, six times, or seven times a week. In a particular embodiment, the treatment is twice a week. In another embodiment, whether an agent is an inhibitor of the IL-25:IL-25R signalling pathway is measured by determining whether the agent is able to reduce the incidence of tumours in adult Apc^{1322T/+} mice treated with said inhibitor, but has no effect or a reduced effect in adult Apc^{1322T/+} mice that are deficient in IL-25R and/or IL-25. In an embodiment, the adult Apc^{1322T/+} mice may have already developed tumours at the time of the treatment, for instance the mice may be seven weeks old. Treatment of an adult mouse may be treatment from seven weeks of age for a further two, four, seven, 14, 21, 28, or more days. In a particular embodiment, the treatment is for four weeks. The treatment may be one, twice, thrice, four times, five times, six times, or seven times a week. In a particular embodiment, the treatment is twice a week. Further information is disclosed in the Examples herein.

In some embodiments, a saturating amount of the inhibitor of the IL-25:IL-25R signalling pathway can reduce IL-25:IL-25R signalling by at least 50%, at least 60%, at least 70%, at least 80%, at least 85%, at least 90%, at least 95%, at least 98%, at least 99%, at least 99.9%, about 100%, or 100%. In a particular embodiment, a saturating amount of the inhibitor of the IL-25:IL-25R signalling pathway abolishes IL-25:IL-25R signalling.

For the avoidance of doubt, an inhibitor of the IL-25:IL-25R signalling pathway is not an agent that acts globally or generically. For instance, an agent that acts to reduce IL-25:IL-25R signalling due to general cytotoxic effects or due to global effects on cell signalling is not an inhibitor of the IL-25:IL-25R signalling pathway.

The IL-25 may be human IL-25, and the sequence may be as set out below.

(SEQ ID NO: 48)
MYSHWPSCCPSKGQDTSEELLRWSTVPVPPLEPARPNRHPESCRASEDG
PLNSRAISPWRYELDRDLNRLPQDLYHARCLCPHCVSLQTGSHMDPRGN
SELLYHNQTVFYRRPCHGEKGTHKGYCLERRLYRVSLACVCVRPRVMG.

The IL-25R may be human IL-25R, and the sequence may be as set out below. This sequence is isoform 1 (identifier Q9NRM6-1).

(SEQ ID NO: 92)
MSLVLLSLAALCRSAVPREPTVQCGSETGPSPEWMLQHDLIPGDLRDLR
VEPVTTSVATGDYSILMNVSWVLRADASIRLLKATKICVTGKSNFQSYS
CVRCNYTEAFQTQTRPSGGKWTFSYIGFPVELNTVYFIGAHNIPNANMN
EDGPSMSVNFTSPGCLDHIMKYKKKCVKAGSLWDPNITACKKNEETVEV
NFTTTPLGNRYMALIQHSTIIGFSQVFEPHQKKQTRASVVIPVTGDSEG
ATVQLTPYFPTCGSDCIRHKGTVVLCPQTGVPFPLDNNKSKPGGWLPLL
LLSLLVATWVLVAGIYLMWRHERIKKTSFSTTTLLPPIKVLVVYPSEIC
FHHTICYFTEFLQNHCRSEVILEKWQKKKIAEMGPVQWLATQKKAADKV
VFLLSNDVNSVCDGTCGKSEGSPSENSQDLFPLAFNLFCSDLRSQIHLH
KYVVVYFREIDTKDDYNALSVCPKYHLMKDATAFCAELLHVKQQVSAGK
RSQACHDGCCSL

In another embodiment, the IL-25R may be isoform 2 (identifier: Q9NRM6-2), and may have the sequence set out below.

(SEQ ID NO: 93)
MSLVLLSLAALCRSAVPREPTVQCGSETGPSPEWMLQHDLIPGDLRDLR
VEPVTTSVATGDYSILMNVSWVLRADASIRLLKATKICVTGKSNFQSYS
CVRCNYTEAFQTQTRPSGGKWTFSYIGFPVELNTVYFIGAHNIPNANMN
EDGPSMSVNFTSPGCLDHIMKYKKKCVKAGSLWDPNITACKKNEETVEV
NFTTTPLGNRYMALIQHSTIIGFSQVFEPHQKKQTRASVVIPVTGDSEG
ATVQVKFSELLWGGKGHRRLFHHSLLLRMSSLLSNALLPADTS

Alleles of IL-25 and IL-25R are also encompassed by the present disclosure. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridisation, amplification and/or database sequence comparison).

The inhibitor may act by binding to IL-25 and sterically blocking, preventing, or reducing the interaction with IL-25R. The inhibitor may act by binding to IL-25R and sterically blocking, preventing, or reducing the interaction with IL-25. For instance, the inhibitor may bind to a region of IL-25 that interacts with IL-25R, or the inhibitor May bind to a region of IL-25R that interacts with IL-25. Alternatively, the inhibitor may bind to either IL-25 or IL-25R in any position, and may lead to a conformational change that prevents or reduces the IL-25:IL-25R interaction.

The IL-25 and/or IL-25R upon which the inhibitor acts may be human IL-25 and/or human IL-25R. In some embodiments, the inhibitor is able to act upon cynomolgus monkey IL-25 and/or murine IL-25, as well as human IL-25. In some embodiments, the inhibitor is able to act upon cynomolgus monkey IL-25R and/or murine IL-25R, as well as human IL-25R. In other embodiments, the inhibitor acts upon the IL-25 and/or IL-25R found within the subject to be treated.

The inhibitor of the IL-25:IL-25R signalling pathway may bind one or more amino acid sequences selected from the group consisting of amino acid residues 46-63 of SEQ ID NO: 48, amino acid residues 66-84 of SEQ ID NO: 48, and amino acid residues 129-135 of SEQ ID NO: 48. In particular embodiment, the inhibitor may bind to amino acid residues 56-63 of SEQ ID NO: 48 and amino acid residues 66-74 of SEQ ID NO: 48.

In some embodiments, the inhibitor of the IL-25:IL-25R signalling pathway competes for binding with any IL-25 or IL-25R binding molecule disclosed herein. The inhibitor may bind to the same epitope, or bind to an overlapping epitope, compared to any IL-25 or IL-25R binding molecule disclosed herein. In particular embodiments, the inhibitor of the IL-25:IL-25R signalling pathway competes for binding with, binds to the same epitope as, or binds to an overlapping epitope with any one of the antibodies: 2C3, any humanised 2C3, RH2.5_R71V, RH2.5_S30T, RH2.5, M6, D9.2, or any humanised D9.2. Two binding molecules may be considered to “compete” when the binding of one to a target is capable of detectably decreasing the binding of the other to the same target.

The inhibitor may be or may comprise an anti-IL-25 antibody, derivative, or functional fragment thereof. The inhibitor may be or may comprise an anti-IL-25R antibody, derivative, or functional fragment thereof. An anti-IL-25 or anti-IL-25R antibody is an antibody capable of specifically binding to its target. “Specifically binding” may mean that the antibody does not bind to the target due to only non-specific interactions, and this property can be determined by comparison to an isotype control or similar. Specific binding does not necessarily require, although it may include, exclusive binding to a single target.

Thus, in an aspect of the invention, there is provided an anti-IL-25 antibody, derivative, or functional fragment thereof for use in a method of treating, preventing, or ameliorating cancer of the intestine.

In another aspect of the invention, there is provided an anti-IL-25R antibody, derivative, or functional fragment thereof for use in a method of treating, preventing, or ameliorating cancer of the intestine.

The anti-IL-25 antibody or anti-IL-25R antibody may be a monoclonal or a polyclonal antibody. The anti-IL-25 antibody or anti-IL-25R antibody may be referred to as a neutralising or blocking antibody. The inhibitor of the IL-25:IL-25R signalling pathway may be an antibody that is: human, i.e. having an amino acid sequence corresponding to that of an antibody produced by a human and/or which have been made using any of the techniques for making human antibodies; humanised, i.e. derived from a parent antibody generated using a non-human system and modified to contain minimal human sequences; or chimeric, i.e. a fusion including regions from more than one species.

A functional fragment or derivative of any agent or antibody disclosed herein may be a part of said agent or antibody that retains the ability to specifically bind to a given target, and so is an antigen-binding fragment. Functionality does not necessarily require, although it may include, retention of the same binding affinity. An antibody fragment may retain the six CDRs of the parent antibody. A fragment or derivative of any antibody disclosed herein may, for instance, be a Fab fragment (e.g. Fab, Fab′, or F(ab′)2), an Fv fragment, an Fd fragment, a single chain variable fragment (scFv), a minibody (e.g. VL-VH-CH3), a diabody (e.g. (scFv)₂ or sc (Fv)₂), a single-domain antibody (e.g. VH or VL), triabody, a tetrabody, or any suitable arrangement or fragment. The inhibitor may also have specificity for another antigen, and hence bispecific antibodies, functional fragments, and derivatives are envisaged. The term derivative encompasses antibodies that have undergone humanisation, further humanisation, affinity maturation, fusion to a modified constant domain, or fusion to an antibody constant domain of a different antibody subtype or of a different species. Thus, the inhibitor of the IL-25:IL-25R signalling pathway may be or may comprise any of the fragments disclosed herein, and may be any derivative of an antibody disclosed herein.

The affinity of an antibody, functional fragment, or derivative described herein is the extent or strength of binding to an epitope. Affinity may be expressed as the dissociation constant, K_{D}. The K_{D} may be measured under standard conditions, for instance in a Biacore assay.

An inhibitor of the IL-25:IL-25R signalling pathway may bind to its target with an equilibrium dissociation constant or KD of less than 5×10⁶ M, less than 1×10⁶ M, less than 5×10⁷ M, less than 1×10⁷ M, less than 5×10⁸ M, less than 1×10⁸ M, less than 5×10⁹ M, less than 1×10⁹ M, less than 5×10¹⁰ M, less than 1×10¹⁰ M, less than 5×10¹¹ M, less than 1×10¹¹ M, less than 5×10¹² M, or less than 1×10¹² M.

An inhibitor of the IL-25:IL-25R signalling pathway that binds to IL-25, such as an anti-IL-25 antibody, may have a K_{D} that is similar to that of any anti-IL-25 antibody disclosed herein, including 2C3, humanised 2C3, RH2.5_R71V, RH2.5_S30T, RH2.5, or M6. A similar K_{D} may be the same order of magnitude, or may be 10% higher or lower.

An inhibitor of the IL-25:IL-25R signalling pathway that binds to IL-25, such as an anti-IL-25R antibody, may have a K_{D} that is similar to that of any anti-IL25R antibody disclosed herein, including D9.2 and humanised variants. A similar K_{D} may be the same order of magnitude, or may be 10% higher or lower.

An antibody, functional fragment, or derivative described herein may be modified so as to be more suitable for the purposes disclosed herein. For instance, a constant region may be chosen or modified so as to minimise effects such as antibody-dependent cell-mediated cytotoxicity, complement fixation, or binding to inhibitory and/or activating Fc receptors. An antibody, functional fragment, or derivative described herein may be modified so as to have an improved half-life, or improved stability in a particular tissue or environment such as the intestine.

The inhibitor of the IL-25:IL-25R signalling pathway may be an antibody of any class, such as: IgA, IgD, IgE, IgG, or IgM, or subclass (isotype), e.g., IgG1, IgG2, IgG3, IgG4, IgA1, or IgA2. The relevant constant regions may be combined with any VH or VL domains disclosed herein, including VH or VL domains defined only by the complementarity determining regions (CDRs).

The inhibitor of the IL-25:IL-25R signalling pathway may be or may comprise a monobody or functional fragment or derivative thereof, and hence may be based upon a fibronectin type III domain scaffold.

The inhibitor of the IL-25:IL-25R signalling pathway may be or may comprise any antibody mimetic, or a functional fragment or derivative thereof. The antibody mimetic may, for example, be any described in Yu et al. including adnectins (monobodies), affibodies, affilins, affimers, affitins, alphabodies, anticalins, aptamers, armadillo repeat protein-based scaffolds, atrimers, avimers, darpins, fynomers, knottins, kunitz domain peptides, or nanofitins (Yu, Yang, Dikici, Deo, and Daunert; Annu Rev Anal Chem (Palo Alto Calif). 2017 Jun. 12; 10 (1): 293-320, incorporated herein by reference in its entirety).

Thus, in another aspect of the invention, there is provided an antibody mimetic, or a functional fragment or derivative thereof for use in a method of treating, preventing, or ameliorating cancer of the intestine, wherein the antibody mimetic, functional fragment, or derivative thereof specifically binds to IL-25.

Thus, in another aspect of the invention, there is provided an antibody mimetic, or a functional fragment or derivative thereof for use in a method of treating, preventing, or ameliorating cancer of the intestine, wherein the antibody mimetic, functional fragment, or derivative thereof specifically binds to IL-25R.

The inhibitor of the IL-25:IL-25R signalling pathway may be based upon IL-25R itself. For instance, a soluble IL-25R capable of binding, and hence sequestering, IL-25. Such soluble IL-25R molecules may comprise an Fc domain.

In some embodiments, the inhibitor of the IL-25:IL-25R signalling pathway does not have a direct cytotoxic effect on IL-25R⁺ cancer cells or IL-25R⁺ cancer stem cells. For instance, in some embodiments the inhibitor is not an antibody-drug conjugate capable of inducing cell death in IL-25R⁺ colorectal cancer stem cells. In some embodiments, the inhibitor of the IL-25:IL-25R signalling pathway has little to no effect on Tuft cell numbers. In some embodiments, the inhibitor of the IL-25:IL-25R signalling pathway reduces Tuft cell numbers by no more than 5%, 10%, 20%, 40%, or 50%.

In a particular embodiment, the inhibitor of the IL-25:IL-25R signalling pathway is or comprises the antibody “2C3” or any antibody or binding molecule, or functional fragment or derivative thereof, as defined in WO2008/129263, EP2144934B1, U.S. Pat. No. 8,206,717B2, or any member of said patent family (each herein incorporated by reference in its entirety).

The inhibitor of the IL-25:IL-25R signalling pathway may be or may comprise an antibody, or functional fragment or derivative thereof, that comprises the CDRs of 2C3. The CDRs may be determined by any technique or method, or a combination of such methods. The CDRs may be determined by any method discussed herein.

The CDRs of 2C3, determined according to Kabat, are as follows:

2c3 VH CDR 1:
(SEQ ID NO: 1)
DYTMN;
or
(SEQ ID NO: 2)
YTMN
203 VH CDR 2:
(SEQ ID NO: 3)
LINPYNGGTSYNQNFKG
2c3 VH CDR 3:
(SEQ ID NO: 4)
EGYDGYLYFAMDY
2c3 VL CDR 1:
(SEQ ID NO: 5)
SASQGISNYLNWYQQ;
or
(SEQ ID NO: 6)
SASQGISNYLN
23 VL CDR 2:
(SEQ ID NO: 7)
YTSSLHS
2c3 VL CDR 3:
(SEQ ID NO: 8)
QQYSKLPYT;
or
(SEQ ID NO: 9)
QQYSKLPYTF

The VH domain of 2C3 may have a sequence as follows:

(SEQ ID NO: 10)
EVQLQQSGPELVKPGASMKISCKASGYSFTDYTMNWVKQSHGKNLEWIG
LINPYNGGTSYNQNFKGKATLTVDKSSSTAYMELLSLTSEDSAVYYCAR
EGYDGYLYFAMDYWGQGTSVTVS;
or
(SEQ ID NO: 22)
EVQLQQSGPELVKPGASMKISCKASGYSFTDYTMNWVKQSHGKNLEWIG
LINPYNGGTSYNQNFKGKATLTVDKSSSTAYMELLSLTSEDSAVYYCAR
EGYDGYLYFAMDYWGQGTSVTVSS

The VL domain of 2C3 may have a sequence as follows:

(SEQ ID NO: 11)
DIQMTQTTSSLSASLGDRVTISCSASQGISNYLNWYQQKADGTVELLIY
YTSSLHSGVPSRFSGSGSGTDYSLTISNLEPEDIATYYCQQYSKLPYTF
GGGTKLEIK.

Thus, in an embodiment, the inhibitor of the IL-25:IL-25R signalling pathway is capable of binding to IL-25, and comprises:
an antibody VH domain, or functional fragment or derivative thereof, comprising:
   a VH CDR1 comprising the amino acid sequence shown in SEQ ID NO: 1 or SEQ ID NO: 2;
   a VH CDR2 comprising the amino acid sequence shown in SEQ ID NO: 3; and
   a VH CDR3 comprising the amino acid sequence shown in SEQ ID NO: 4; and
an antibody a VL domain, or functional fragment or derivative thereof, comprising:
   a VL CDR1 comprising the amino acid sequence shown in SEQ ID NO: 5 or SEQ ID NO: 6;
   a VL CDR2 comprising the amino acid sequence shown in SEQ ID NO: 7; and
   a VL CDR3 comprising the amino acid sequence shown in SEQ ID NO: 8 or SEQ ID NO: 9.

Therefore, in an aspect of the invention, there is provided an anti-IL-25 antibody for use in a method of treating, preventing, or ameliorating cancer of the intestine, wherein the anti-IL-25 antibody comprises:
an antibody VH domain, or functional fragment or derivative thereof, comprising:
   a VH CDR1 comprising the amino acid sequence shown in SEQ ID NO: 1 or SEQ ID NO: 2;
   a VH CDR2 comprising the amino acid sequence shown in SEQ ID NO: 3; and
   a VH CDR3 comprising the amino acid sequence shown in SEQ ID NO: 4; and
an antibody a VL domain, or functional fragment or derivative thereof, comprising:
   a VL CDR1 comprising the amino acid sequence shown in SEQ ID NO: 5 or SEQ ID NO: 6;
   a VL CDR2 comprising the amino acid sequence shown in SEQ ID NO: 7; and
   a VL CDR3 comprising the amino acid sequence shown in SEQ ID NO: 8 or SEQ ID NO: 9.

The above recited CDRs may comprise one, two, or three amino acid substitutions compared to the recited sequence, which may be conservative substitutions. The substitutions may be such that the properties of the inhibitor are not affected, or are not negatively affected. For instance, binding affinity may be retained or improved.

In some embodiments, the inhibitor of the IL-25:IL-25R signalling pathway comprises an antibody VH domain comprising the amino acid sequence shown in SEQ ID NO: 10 or SEQ ID NO: 22, optionally comprising at least one, from one to ten, from one to five, from one to three, or one, two, three, four, five, six, seven, eight, nine, or ten amino acid substitutions. The substitutions may be in residues that are not within a CDR. In some embodiments, said amino acid substitutions are conservative amino acid substitutions. The substitutions may be such that the properties of the inhibitor are not affected, or are not negatively affected. For instance, binding affinity may be retained or improved. In a particular embodiment, the domain comprises from one to three conservative amino acid substitutions.

In some embodiments, the inhibitor of the IL-25:IL-25R signalling pathway comprises an antibody VL domain comprising the amino acid sequence shown in SEQ ID NO: 11, optionally comprising at least one, from one to ten, from one to five, from one to three, or one, two, three, four, five, six, seven, eight, nine, or ten amino acid substitutions. The substitutions may be in residues that are not within a CDR. In some embodiments, said amino acid substitutions are conservative amino acid substitutions. The substitutions may be such that the properties of the inhibitor are not affected, or are not negatively affected. For instance, binding affinity may be retained or improved. In a particular embodiment, the domain comprises from one to three conservative amino acid substitutions.

In a particular embodiment, the inhibitor of the IL-25:IL-25R signalling pathway is capable of binding to IL-25, and comprises an antibody VH domain comprising the amino acid sequence shown in SEQ ID NO: 22, and an antibody VL domain comprising the amino acid sequence shown in SEQ ID NO: 11.

Thus, in a particular aspect of the invention, there is provided an anti-IL-25 antibody for use in a method of treating, preventing, or ameliorating cancer of the intestine, wherein the anti-IL-25 antibody comprises an antibody VH domain comprising the amino acid sequence shown in SEQ ID NO: 22 and an antibody VL domain comprising the amino acid sequence shown in SEQ ID NO: 11.

In another embodiment, the inhibitor of the IL-25:IL-25R signalling pathway may comprise a target binding member that binds IL-25 and which comprises an antibody VH domain comprising a VH CDR3 with the amino acid sequence of SEQ ID NO: 4. In more specific embodiments, the target binding member is a VH domain which comprises a VH CDR3 of SEQ ID NO: 4 together with a CDR1 of SEQ ID NO: 1 and a CDR2 of SEQ ID NO: 3. The VH domain may have human framework regions, or the framework regions shown in SEQ ID NO: 22. The VH domains may be paired with a VL domain of the invention, e.g. a VL domain with a CDR1 of SEQ ID NO: 5, a CDR2 of SEQ ID NO: 7, and a CDR3 of SEQ ID NO: 8. These CDRs may be in a VL domain having human framework regions or may be the VL domain of SEQ ID NO: 11. Thus in a particular embodiment, the target binding member may bind IL-25 and comprise the 2C3 VH domain (SEQ ID NO: 22) and/or the 2C3 VL domain (SEQ ID NO: 11).

The inhibitor of the IL-25:IL-25R signalling pathway may be or may comprise an antibody, or fragment or derivative thereof, that binds IL-25 and which comprises an antibody VH domain or a substantial portion thereof comprising a VH CDR1 having an amino acid sequence as set out in SEQ ID NO: 1; a VH CDR2 having an amino acid sequence as set out in SEQ ID NO: 3; and a VH CDR3 having an amino acid sequence as set out in SEQ ID NO: 4, and which further comprises a VL domain or a substantial portion thereof comprising a CDR1 having an amino acid sequence as set out in SEQ ID NO: 5, a CDR2 having an amino acid sequence as set out in SEQ ID NO: 7; and a CDR3 having an amino acid sequence as set out in SEQ ID NO: 8. Optionally, the VH domain or substantial portion thereof comprises a human framework region or the 2C3 amino acid sequence as set out in SEQ ID NO: 22. Optionally, the VL domain comprises a human framework region or the 2C3 amino acid sequence as set out in SEQ ID NO: 11. The 2C3 antibody may, in some embodiments, comprise an IgG1 or IgG4 constant region.

In another particular embodiment, the inhibitor of the IL-25:IL-25R signalling pathway is or comprises a humanised form of the antibody 2C3 or any antibody or binding molecule, or functional fragment or derivative thereof, as defined in WO2010/038155, EP2344538B1, U.S. Pat. No. 8,658,169B2, or any member of said patent family (each herein incorporated by reference in its entirety). In particular, the inhibitor may be, or may comprise, the antibodies RH2.5_R71V, RH2.5_S30T, or RH2.5, or a functional fragment or derivative thereof.

The inhibitor of the IL-25:IL-25R signalling pathway may be or may comprise an antibody, or functional fragment or derivative thereof, that comprises the CDRs of a humanised 2C3 as defined in the WO2010/038155 patent family, for instance RH2.5_R71V, RH2.5_S30T, or RH2.5. The CDRs may be determined by any technique or method, or a combination of such methods. The CDRs may be determined by any method discussed herein.

The CDRs of humanised 2C3, according to one method of determination, may have any of the following sequences.

Humanised 2c3 VH CDR 1:
SEQ ID NO: 1
;
SEQ ID NO: 2
;
(SEQ ID NO: 12)
GYTMN;
(SEQ ID NO: 13)
AYTMN;
(SEQ ID NO: 14)
SYTMN;
(SEQ ID NO: 15)
VYTMN;
(SEQ ID NO: 16)
NYTMN;
(SEQ ID NO: 17)
KYTMN;
(SEQ ID NO: 18)
YYTMN;
or
(SEQ ID NO: 19)
MYTMN
Humanised 2c3 VH CDR 2:
SEQ ID NO: 3
Humanised 2c3 VH CDR 3:
SEQ ID NO: 4
;
(SEQ ID NO: 20)
EDYDGYLYFAMDY;
or
(SEQ ID NO: 21)
ENYDGYLYFAMDY
Humanised 2c3 VL CDR 1:
SEQ ID NO: 5
;
or
SEQ ID NO: 6
Humanised 2c3 VL CDR 2:
SEQ ID NO: 7
Humanised 2c3 VL CDR 3:
SEQ ID NO: 8
;
or
SEQ ID NO: 9

The VH domain of humanised 2C3, or of a 2C3 derivative, may have any of the following sequences:

EVQLVESGAEVKKPGASVKVSCKASGYSFX₁X₂YTMNWVRQAPGQRLEW
X₃GLINPYNGGTSYNQNFKGRVTLTX₄DTSASTAYLELNSLRSEDTGVYY
CAREX₅YDGYLYFAMDYWGQGTLVTVSS

wherein:
X₁ is S or T;
X₂ is G, D, A, S, V, N, K, Y or M;
X₃ is M or I;
X₄ is V or R; and
X₅ is D, N or G (SEQ ID NO: 24);

(SEQ ID NO: 25)
EVQLVESGAEVKKPGASVKVSCKASGYSFSGYTMNWVRQAPGQRLEWMG
LINPYNGGTSYNQNFKGRVTLTVDTSASTAYLELNSLRSEDTGVYYCAR
EDYDGYLYFAMDYWGQGTLVTVSS;
(SEQ ID NO: 26)
EVQLVESGAEVKKPGASVKVSCKASGYSFTGYTMNWVRQAPGQRLEWMG
LINPYNGGTSYNQNFKGRVTLTRDTSASTAYLELNSLREDTGVYYCARE
DYDGYLYFAMDYWGQGTLVVSS;
(SEQ ID NO: 27)
EVQLVESGAEVKKPGASVKVSCKASGYSFSGYTMNWVRQAPGQRLEWMG
LINPYNGGTSYNQNFKGRVTLTRDTSASTAYLELNSLRSEDTGVYYCAR
EDYDGYLYFAMDYWGQGTLVTVSS;
(SEQ ID NO: 28)
EVQLVESGAEVKKPGASVKVSCKASGYSFTGYTMNWVRQAPGQRLEWMG
LINPYNGGTSYNQNFKGRVTLTVDTSASTAYLELNSLRSEDTGVYYCAR
EDYDGYLYFAMDYWGQGTLVTVSS;
(SEQ ID NO: 29)
EVQLVESGAEVKKPGASVKVSCKASGYSFSDYTMNWVRQAPGQRLEWMG
LINPYNGGTSYNQNFKGRVTLTVDTSASTAYLELNSLRSEDTGVYYCAR
EDYDGYLYFAMDYWGQGTLVTVSS;
(SEQ ID NO: 30)
EVQLVESGAEVKKPGASVKVSCKASGYSFTDYTMNWVRQAPGQRLEWMG
LINPYNGGTSYNQNFKGRVTLTRDTSASTAYLELNSLRSEDTGVYYCAR
EDYDGYLYFAMDYWGQGTLVTVSS;
(SEQ ID NO: 31)
EVQLVESGAEVKKPGASVKVSCKASGYSFTDYTMNWVRQAPGQRLEWMG
LINPYNGGTSYNQNFKGRVTLTVDTSASTAYLELNSLRSEDTGVYYCAR
EDYDGYLYFAMDYWGQGTLVTVSS;
(SEQ ID NO: 32)
EVQLVESGAEVKKPGASVKVSCKASGYSFSGYTMNWVRQAPGQRLEWIG
LINPYNGGTSYNQNFKGRVTLTVDTSASTAYLELNSLRSEDTGVYYCAR
EDYDGYLYFAMDYWGQGTLVTVSS;
(SEQ ID NO: 33)
EVQLVESGAEVKKPGASVKVSCKASGYSFTGYTMNWVRQAPGQRLEWIG
LINPYNGGTSYNQNFKGRVTLTRDTSASTAYLELNSLRSEDTGVYYCAR
EDYDGYLYFAMDYWGQGTLVTVSS;
(SEQ ID NO: 34)
EVQLVESGAEVKKPGASVKVSCKASGYSFTGYTMNWVRQAPGQRLEWIG
LINPYNGGTSYNQNFKGRVTLTVDTSASTAYLELNSLRSEDTGVYYCAR
EDYDGYLYFAMDYWGQGTLVTVSS;
(SEQ ID NO: 35)
EVQLVESGAEVKKPGASVKVSCKASGYSFSDYTMNWVRQAPGQRLEWIG
LINPYNGGTSYNQNFKGRVTLTVDTSASTAYLELNSLRSEDTGVYYCAR
EDYDGYLYFAMDYWGQGTLVTVSS;
(SEQ ID NO: 36)
EVQLVESGAEVKKPGASVKVSCKASGYSFTDYTMNWVRQAPGQRLEWIG
LINPYNGGTSYNQNFKGRVTLTRDTSASTAYLELNSLRSEDTGVYYCAR
EDYDGYLYFAMDYWGQGTLVTVSS;
(SEQ ID NO: 37)
EVQLVESGAEVKKPGASVKVSCKASGYSFTDYTMNWVRQAPGQRLEWIG
LINPYNGGTSYNQNFKGRVTLTVDTSASTAYLELNSLRSEDTGVYYCAR
EDYDGYLYFAMDYWGQGTLVTVSS;
or
(SEQ ID NO: 38)
EVRLVQSGAEVKKPGESLKISCKASGYSFTDYTMNWVRQMPGKGLEWIG
LINPYNGGTSYNQNFKGQVTISVDKSINTAYLQWSSLKATDTAMYYCAR
EGYDGYLYFAMDYWGQGTLVIVSS.

The VL domain of humanised 2C3, or of a 2C3 derivative, may have any of the following sequences:

SEQ ID NO: 11
MRVPAQLLGLLLLWLPDTRCDIQMTQSPSSLSASVGDRVTITCSASQGI
SNYLNWYQQKPGKVPKLLIYYTSSLHSGVPSRFSGSGSGTDFTLTISSL
QPEDVATYYCQQYSKLPYTFGQGTKLEIK (SEQ ID NO: 40;
includes leader sequence)
(SEQ ID NO: 41)
DIQMTQSPSSLSASVGDRVTITCSASQGISNYLNWYQQKPGKVPKLLIY
YTSSLHSGVPSRFSGSGSGTDFTLTISSLQPEDVATYYCQQYSKLPYTF
GQGTKLEIK

Thus, in an embodiment, the inhibitor of the IL-25:IL-25R signalling pathway is capable of binding to IL-25, and comprises:
an antibody VH domain, or functional fragment or derivative thereof, comprising:
   a VH CDR1 comprising the amino acid sequence shown in any one of SEQ ID NO: 1, SEQ ID NO: 2, SEQ ID NO: 12, SEQ ID NO: 13, SEQ ID NO: 14, SEQ ID NO: 15, SEQ ID NO: 16, SEQ ID NO: 17, SEQ ID NO: 18, or SEQ ID NO: 19;
   a VH CDR2 comprising the amino acid sequence shown in SEQ ID NO: 3; and
   a VH CDR3 comprising the amino acid sequence shown in any one of SEQ ID NO: 4, SEQ ID NO: 20, or SEQ ID NO: 21; and
an antibody a VL domain, or functional fragment or derivative thereof, comprising:
   a VL CDR1 comprising the amino acid sequence shown in SEQ ID NO: 5 or SEQ ID NO: 6;
   a VL CDR2 comprising the amino acid sequence shown in SEQ ID NO: 7; and
   a VL CDR3 comprising the amino acid sequence shown in SEQ ID NO: 8 or SEQ ID NO: 9.

Therefore, in an aspect of the invention, there is provided an anti-IL-25 antibody for use in a method of treating, preventing, or ameliorating cancer of the intestine, wherein the anti-IL-25 antibody comprises:
an antibody VH domain, or functional fragment or derivative thereof, comprising:
   a VH CDR1 comprising the amino acid sequence shown in any one of SEQ ID NO: 1, SEQ ID NO: 2, SEQ ID NO: 12, SEQ ID NO: 13, SEQ ID NO: 14, SEQ ID NO: 15, SEQ ID NO: 16, SEQ ID NO: 17, SEQ ID NO: 18, or SEQ ID NO: 19;
   a VH CDR2 comprising the amino acid sequence shown in SEQ ID NO: 3; and
   a VH CDR3 comprising the amino acid sequence shown in any one of SEQ ID NO: 4, SEQ ID NO: 20, or SEQ ID NO: 21; and
an antibody a VL domain, or functional fragment or derivative thereof, comprising:
   a VL CDR1 comprising the amino acid sequence shown in SEQ ID NO: 5 or SEQ ID NO: 6;
   a VL CDR2 comprising the amino acid sequence shown in SEQ ID NO: 7; and
   a VL CDR3 comprising the amino acid sequence shown in SEQ ID NO: 8 or SEQ ID NO: 9.

The above recited CDRs may comprise one, two, or three amino acid substitutions compared to the recited sequence, which may be conservative substitutions. The substitutions may be such that the properties of the inhibitor are not affected, or are not negatively affected. For instance, binding affinity may be retained or improved.

In another embodiment, the inhibitor of the IL-25:IL-25R signalling pathway is capable of binding to IL-25, and comprises:
an antibody VH domain, or functional fragment or derivative thereof, comprising:
   a VH CDR1 comprising the amino acid sequence shown in SEQ ID NO: 12;
   a VH CDR2 comprising the amino acid sequence shown in SEQ ID NO: 3; and
   a VH CDR3 comprising the amino acid sequence shown in SEQ ID NO: 20; and
an antibody a VL domain, or functional fragment or derivative thereof, comprising:
   a VL CDR1 comprising the amino acid sequence shown in SEQ ID NO: 5 or SEQ ID NO: 6;
   a VL CDR2 comprising the amino acid sequence shown in SEQ ID NO: 7; and
   a VL CDR3 comprising the amino acid sequence shown in SEQ ID NO: 8 or SEQ ID NO: 9.

In another embodiment, the inhibitor of the IL-25:IL-25R signalling pathway comprises an antibody VH domain comprising the amino acid sequence shown in any one of SEQ ID NOS: 10, 24, 25, 26, 27, 28, 29, 30, 31, 32, 33, 34, 35, 36, 37, or 38, optionally comprising at least one, from one to ten, from one to five, from one to three, or one, two, three, four, five, six, seven, eight, nine, or ten amino acid substitutions. The substitutions may be in residues that are not within a CDR. In some embodiments, said amino acid substitutions are conservative amino acid substitutions. The substitutions may be such that the properties of the inhibitor are not affected, or are not negatively affected. For instance, binding affinity may be retained or improved. In a particular embodiment, the domain comprises from one to three conservative amino acid substitutions.

In another embodiment, the inhibitor of the IL-25:IL-25R signalling pathway comprises an antibody VL domain comprising the amino acid sequence shown in SEQ ID NO: 11 or SEQ ID NO: 41, optionally comprising at least one, from one to ten, from one to five, from one to three, or one, two, three, four, five, six, seven, eight, nine, or ten amino acid substitutions. The substitutions may be in residues that are not within a CDR. In some embodiments, said amino acid substitutions are conservative amino acid substitutions. The substitutions may be such that the properties of the inhibitor are not affected, or are not negatively affected. For instance, binding affinity may be retained or improved. In a particular embodiment, the domain comprises from one to three conservative amino acid substitutions.

In a particular embodiment, the inhibitor of the IL-25:IL-25R signalling pathway is capable of binding to IL-25, and comprises an antibody VH domain comprising the amino acid sequence shown in SEQ ID NO: 24, and an antibody VL domain comprising the amino acid sequence shown in SEQ ID NO: 11 or SEQ ID NO: 41.

In a particular embodiment, the inhibitor of the IL-25:IL-25R signalling pathway is capable of binding to IL-25, and comprises an antibody VH domain comprising the amino acid sequence shown in SEQ ID NO: 25, and an antibody VL domain comprising the amino acid sequence shown in SEQ ID NO: 41.

In a particular embodiment, the inhibitor of the IL-25:IL-25R signalling pathway is capable of binding to IL-25, and comprises an antibody VH domain comprising the amino acid sequence shown in SEQ ID NO: 26, and an antibody VL domain comprising the amino acid sequence shown in SEQ ID NO: 41.

Thus, in a particular aspect of the invention, there is provided an anti-IL-25 antibody for use in a method of treating, preventing, or ameliorating cancer of the intestine, wherein the anti-IL-25 antibody comprises an antibody VH domain comprising the amino acid sequence shown in any one of SEQ ID NOs: 24, 25, or 26, and an antibody VL domain comprising the amino acid sequence shown in any one of SEQ ID NOs: 11 or 41.

In an embodiment, the inhibitor of the IL-25:IL-25R signalling pathway may be a target binding member that binds IL-25, and comprises an antibody VH domain which comprises SEQ ID NO:24:

Glu Val Gln Leu Val Glu Ser Gly Ala Glu Val Lys
Lys Pro Gly Ala Ser Val Lys Val
Ser Cys Lys Ala Ser Gly Tyr Ser Phe Xa1 Xa2 Tyr
Thr Met Asn Trp Val Arg Gln
Ala Pro Gly Gln Arg Leu Glu Trp Xa3 Gly Leu Ile
Asn Pro Tyr Asn Gly Gly Thr
Ser Tyr Asn Gln Asn Phe Lys Gly Arg Val Thr Leu
Thr Xa4 Asp Thr Ser Ala Ser
Thr Ala Tyr Leu Glu Leu Asn Ser Leu Arg Ser Glu
Asp Thr Gly Val Tyr Tyr Cys
Ala Arg Glu Xa5 Tyr Asp Gly Tyr Leu Tyr Phe Ala
Met Asp Tyr Trp Gly Gln Gly
Thr Leu Val Thr Val Ser Ser

wherein:
Xa1 is Ser or Thr;
Xa2 is Gly, Asp, Ala, Ser, Val, Asn, Lys, Tyr or Met;
Xa3 is Met or Ile;
Xa4 is Val or Arg; and
Xa5 is Asp, Asn or Gly; optionally further comprising an antibody VL domain which comprises Kabat CDRs 1-3 as set out as residues 24-34 (SEQ ID NO:6); 50-56 (SEQ ID NO:7) and 89-97 (SEQ ID NO:8) respectively of SEQ ID NO: 11.

In some embodiments:
(a) Xa2 is Gly and Xa5 is Asp or Asn;
(b) Xa2 is Gly and Xa5 is Asp
(c) Xa1 is Ser;
(d) Xa1 is Thr;
(e) Xa3 is Met;
(f) Xa3 is Ile;
(g) Xa4 is Val; or
(h) Xa4 is Arg.

In some embodiments, the residues Xa1-Xa5 are in the following combinations:

Kabat Res:

| | | | | |
|---|---|---|---|---|
| 30 | 31 | 48 | 71 | 96 |

Position in SEQ ID NO: 24

| | | | | |
|---|---|---|---|---|
| 30 | 31 | 48 | 72 | 100 |

Seq List Res:

| | Xa1 | Xa2 | Xa3 | Xa4 | Xa5 |
|---|---|---|---|---|---|
| SEQ ID NO: 25 | Ser | Gly | Met | Val | Asp |
| SEQ ID NO: 26 | Thr | Gly | Met | Arg | Asp |
| SEQ ID NO: 27 | Ser | Gly | Met | Arg | Asp |
| SEQ ID NO: 28 | Thr | Gly | Met | Val | Asp |
| SEQ ID NO: 29 | Ser | Asp | Met | Val | Asp |
| SEQ ID NO: 30 | Thr | Asp | Met | Arg | Asp |
| SEQ ID NO: 31 | Thr | Asp | Met | Val | Asp |
| SEQ ID NO: 32 | Ser | Gly | Ile | Val | Asp |
| SEQ ID NO: 33 | Thr | Gly | Ile | Arg | Asp |
| SEQ ID NO: 34 | Thr | Gly | Ile | Val | Asp |
| SEQ ID NO: 35 | Ser | Asp | Ile | Val | Asp |
| SEQ ID NO: 36 | Thr | Asp | Ile | Arg | Asp |
| SEQ ID NO: 37 | Thr | Asp | Ile | Val | Asp |

In some embodiments:
(a) the VL domain further includes residues 35-38 of SEQ ID NO:11 adjacent to CDR1 (SEQ ID NO:6);
(b) the target binding member comprises SEQ ID NO:11;
(c) the VL domain is humanised; or
(d) the VL domain is humanised, and the sequence of the target binding member comprises amino acids 21-127 of SEQ ID NO:40.

The humanised 2C3 antibody may comprise a constant region, for instance an IgG1 or IgG4 constant region.

In another particular embodiment, the inhibitor of the IL-25:IL-25R signalling pathway is or comprises a humanised form of the antibody 2C3 or any antibody or binding molecule, or functional fragment or derivative thereof, as defined in WO2011/123507, EP2552961B1, U.S. Pat. No. 8,785,605B2, or any member of said patent family (each herein incorporated by reference in its entirety). In a particular embodiment, the inhibitor is or comprises the antibody M6, or a functional fragment or derivative thereof.

The inhibitor of the IL-25:IL-25R signalling pathway may be or may comprise an antibody, or functional fragment or derivative thereof, that comprises the CDRs of a humanised 2C3, for instance M6, as defined in the WO2011/123507 patent family. The CDRs may be determined by any technique or method, or a combination of such methods. The CDRs may be determined by any method discussed herein.

The CDRs of M6, according to one method of determination, may have any of the following sequences.

M6 VH CDR 1:
SEQ ID NO: 12
M6 VH CDR 2:
SEQ ID NO: 3
M6 VH CDR 3:
SEQ ID NO: 20
M6 VL CDR 1:
SEQ ID NO: 6
M6 VL CDR 2:
SEQ ID NO: 7
M6 VL CDR 3:
(SEQ ID NO: 44)
QQYLAFPYT;
or
(SEQ ID NO: 79)
QQYLAFPYTF

The VH domain of M6 may the following sequence:
SEQ ID NO: 25

The heavy chain of M6 may have the following sequence:

(SEQ ID NO: 45)
EVQLVESGAEVKKPGASVKVSCKASGYSFSGYTMNWVRQAPGQRLEWMG
LINPYNGGTSYNQNFKGRVTLTVDTSASTAYLELNSLRSEDTGVYYCAR
EDYDGYLYFAMDYWGQGTLVTVSSASTKGPSVFPLAPSSKSTSGGTAAL
GCLVKDYFPEPVTVSWNSGALTSGVHTFPAVLQSSGLYSLSSVVTVPSS
SLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISK
AKGQPREPQVYTLPPSRDELTKNQVSLTCLVKGFYPSDIAVEWESNGQP
ENNYKTTPPVLDSDGSFFLYSKLTVDKSRWQQGNVFSCSVMHEALHNHY
TQKSLSLSPGK

The VL domain of M6 may the following sequence:

(SEQ ID NO: 46)
DIQMTQSPSSLSASVGDRVTITCSASQGISNYLNWYQQKPGKVPKLLIY
YTSSLHSGVPSRFSGSGSGTDFTLTISSLQPEDVATYYCQQYLAFPYTF
GQGTKLEIK

The light chain of M6 may have the following sequence:

(SEQ ID NO: 47)
DIQMTQSPSSLSASVGDRVTITCSASQGISNYLNWYQQKPGKVPKLLIY
YTSSLHSGVPSRFSGSGSGTDFTLTISSLQPEDVATYYCQQYLAFPYTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

Thus, in an embodiment, the inhibitor of the IL-25:IL-25R signalling pathway is capable of binding to IL-25, and comprises:
an antibody VH domain, or functional fragment or derivative thereof, comprising:
   a VH CDR1 comprising the amino acid sequence shown in SEQ ID NO: 12;
   a VH CDR2 comprising the amino acid sequence shown in SEQ ID NO: 3; and
   a VH CDR3 comprising the amino acid sequence shown in SEQ ID NO: 20; and
an antibody a VL domain, or functional fragment or derivative thereof, comprising:
   a VL CDR1 comprising the amino acid sequence shown in SEQ ID NO: 6;
   a VL CDR2 comprising the amino acid sequence shown in SEQ ID NO: 7; and
   a VL CDR3 comprising the amino acid sequence shown in SEQ ID NO: 44 or SEQ ID NO: 79.

Therefore, in an aspect of the invention, there is provided an anti-IL-25 antibody for use in a method of treating, preventing, or ameliorating cancer of the intestine, wherein the anti-IL-25 antibody comprises:
an antibody VH domain, or functional fragment or derivative thereof, comprising:
   a VH CDR1 comprising the amino acid sequence shown in SEQ ID NO: 12;
   a VH CDR2 comprising the amino acid sequence shown in SEQ ID NO: 3; and
   a VH CDR3 comprising the amino acid sequence shown in SEQ ID NO: 20; and
an antibody a VL domain, or functional fragment or derivative thereof, comprising:
   a VL CDR1 comprising the amino acid sequence shown in SEQ ID NO: 6;
   a VL CDR2 comprising the amino acid sequence shown in SEQ ID NO: 7; and
   a VL CDR3 comprising the amino acid sequence shown in SEQ ID NO: 44 or SEQ ID NO: 79.

The above recited CDRs may comprise one, two, or three amino acid substitutions compared to the recited sequence, which may be conservative substitutions. The substitutions may be such that the properties of the inhibitor are not affected, or are not negatively affected. For instance, binding affinity may be retained or improved.

In another embodiment, the inhibitor of the IL-25:IL-25R signalling pathway comprises an antibody VH domain comprising the amino acid sequence shown in any one of SEQ ID NOS: 25, optionally comprising at least one, from one to ten, from one to five, from one to three, or one, two, three, four, five, six, seven, eight, nine, or ten amino acid substitutions. The substitutions may be in residues that are not within a CDR. In some embodiments, said amino acid substitutions are conservative amino acid substitutions. The substitutions may be such that the properties of the inhibitor are not affected, or are not negatively affected. For instance, binding affinity may be retained or improved. In a particular embodiment, the domain comprises from one to three conservative amino acid substitutions.

In another embodiment, the inhibitor of the IL-25:IL-25R signalling pathway comprises an antibody VL domain comprising the amino acid sequence shown in SEQ ID NO: 46, optionally comprising at least one, from one to ten, from one to five, from one to three, or one, two, three, four, five, six, seven, eight, nine, or ten amino acid substitutions. The substitutions may be in residues that are not within a CDR. In some embodiments, said amino acid substitutions are conservative amino acid substitutions. The substitutions may be such that the properties of the inhibitor are not affected, or are not negatively affected. For instance, binding affinity may be retained or improved. In a particular embodiment, the domain comprises from one to three conservative amino acid substitutions.

In a particular embodiment, the inhibitor of the IL-25:IL-25R signalling pathway is capable of binding to IL-25, and comprises an antibody VH domain comprising the amino acid sequence shown in SEQ ID NO: 25, and an antibody VL domain comprising the amino acid sequence shown in SEQ ID NO: 46.

Thus, in a particular aspect of the invention, there is provided an anti-IL-25 antibody for use in a method of treating, preventing, or ameliorating cancer of the intestine, wherein the anti-IL-25 antibody comprises an antibody VH domain comprising the amino acid sequence shown in SEQ ID NO: 25, and an antibody VL domain comprising the amino acid sequence shown in SEQ ID NO: 46.

In a particular embodiment, the inhibitor of the IL-25:IL-25R signalling pathway is capable of binding to IL-25, and comprises a polypeptide comprising the amino acid sequence shown in SEQ ID NO: 45, and polypeptide comprising the amino acid sequence shown in SEQ ID NO: 47.

In one embodiment, the inhibitor of the IL-25:IL-25R signalling pathway is or comprises a target binding member that binds IL-25, wherein the target binding member binds one or more amino acid sequences selected from the group consisting of amino acid residues 46-63 of SEQ ID NO: 48, amino acid residues 66-84 of SEQ ID NO: 48 and amino acid residues 129-135 of SEQ ID NO: 48. In a particular embodiment, the target binding member of the invention binds amino acid residues 56-63 of SEQ ID NO: 48 and amino acid residues 66-74 of SEQ ID NO: 48. In another embodiment, the target binding member comprises an antibody VL domain comprising a CDR3 having the amino acid sequence QQYLAFPYTF (SEQ ID NO: 79).

In another embodiment, the inhibitor of the IL-25:IL-25R signalling pathway is or comprises a target binding member that binds IL-25, wherein the target binding member comprises:
a) an antibody VL domain comprising a CDR1 having the amino acid sequence SASQGISNYLN (SEQ ID NO: 6), a CDR2 having the amino acid sequence YTSSLHS (SEQ ID NO: 7) and a CDR3 having the amino acid sequence QQYLAFPYTF (SEQ ID NO: 79); and
b) an antibody VH domain comprising a CDR1 having the amino acid sequence GYTMN (SEQ ID NO: 12), a CDR2 having the amino acid sequence LINPYNGGTSYNQNFKG (SEQ ID NO: 3) and a CDR3 having the amino acid sequence EDYDGYLYFAMDY (SEQ ID NO: 20).

In a particular embodiment, the inhibitor of the IL-25:IL-25R signalling pathway is or comprises a target binding member comprises a VL domain comprising SEQ ID NO: 47 and a VH domain comprising SEQ ID NO: 45. In a further embodiment, the target binding member comprises a whole antibody.

In another embodiment, the inhibitor of the IL-25:IL-25R signalling pathway is or comprises a target binding member that competes for binding to IL-25 with a target binding member that binds one or more amino acid sequences selected from the group consisting of amino acid residues 46-63 of SEQ ID NO: 48, amino acid residues 66-84 of SEQ ID NO: 48 and amino acid residues 129-135 of SEQ ID NO: 48. In a particular embodiment, the target binding member has a binding affinity for human IL-25 that is less than or equal to about 50 pM.

In another embodiment, the inhibitor of the IL-25:IL-25R signalling pathway is or comprises a target binding member of the invention comprising:
a) an antibody VL domain comprising an amino acid sequence having from 1 to about 20 amino acid substitutions relative to the amino acid sequence of SEQ ID NO: 47;
b) an antibody VH domain comprising an amino acid sequence having from 1 to about 20 amino acid substitutions relative to the amino acid sequence of SEQ ID NO: 45; or
c) a combination thereof.

In another embodiment, the inhibitor of the IL-25:IL-25R signalling pathway is or comprises a humanized antibody or antigen binding fragment thereof, that binds IL-25, wherein the antibody or antigen binding fragment thereof comprises:
a) an antibody VL domain comprising a CDR1 having the amino acid sequence SASQGISNYLN (SEQ ID NO: 6), a CDR2 having the amino acid sequence YTSSLHS (SEQ ID NO: 7) and a CDR3 having the amino acid sequence QQYLAFPYTF (SEQ ID NO: 79); and
b) an antibody VH domain comprising a CDR1 having the amino acid sequence GYTMN (SEQ ID NO: 12), a CDR2 having the amino acid sequence LINPYNGGTSYNQNFKG (SEQ ID NO: 3) and a CDR3 having the amino acid sequence EDYDGYLYFAMDY (SEQ ID NO:20), wherein

the antibody or antigen binding fragment thereof has a binding affinity for IL-25 that is less than or equal to 53 pM as determined in a Biacore assay. The binding affinity for IL-25 may be less than or equal to about 50 pM, about 45 pM, about 40 pM, about 35 pM, about 30 pM, about 25 pM, or about 20 pM. The humanized antibody or antigen binding fragment thereof may comprise the VL domain of the antibody light chain of SEQ ID NO: 47. The humanized antibody or antigen binding fragment thereof of may comprise the VH domain of the antibody heavy chain of SEQ ID NO: 45.

In an embodiment, the inhibitor of the IL-25:IL-25R signalling pathway is capable of binding to IL-25, and comprises
an antibody VH domain, or functional fragment or derivative thereof, comprising:
   a VH CDR1 comprising the amino acid sequence shown in any one of SEQ ID NOs: 1, 2, 12, 13, 14, 15, 16, 17, 18, or 19;
   a VH CDR2 comprising the amino acid sequence shown in SEQ ID NO: 3; and
   a VH CDR3 comprising the amino acid sequence shown in any one of SEQ ID NOs: 4, 20, or 21; and
an antibody a VL domain, or functional fragment or derivative thereof, comprising:
   a VL CDR1 comprising the amino acid sequence shown in SEQ ID NO: 5 or SEQ ID NO: 6;
   a VL CDR2 comprising the amino acid sequence shown in SEQ ID NO: 7; and
   a VL CDR3 comprising the amino acid sequence shown in any one of SEQ ID NOs: 8, 9, 44, or 79.

The above recited CDRs may comprise one, two, or three amino acid substitutions compared to the recited sequence, which may be conservative substitutions. The substitutions may be such that the properties of the inhibitor are not affected, or are not negatively affected. For instance, binding affinity may be retained or improved.

In another embodiment, the inhibitor of the IL-25:IL-25R signalling pathway comprises an antibody VH domain comprising the amino acid sequence shown in any one of SEQ ID NOS: 10, 22, 24, 25, 26, 27, 28, 29, 30, 31, 32, 33, 34, 35, 36, 37, or 38, and an antibody VL domain comprising the amino acid sequence shown in any one of SEQ ID NOS: 11, 41, or 47. Optionally the VH and/or VL domain comprises at least one, from one to ten, from one to five, from one to three, or one, two, three, four, five, six, seven, eight, nine, or ten amino acid substitutions. The substitutions may be in residues that are not within a CDR. In some embodiments, said amino acid substitutions are conservative amino acid substitutions. The substitutions may be such that the properties of the inhibitor are not affected, or are not negatively affected. For instance, binding affinity may be retained or improved. In a particular embodiment, the domain comprises from one to three conservative amino acid substitutions.

The leader sequence for the VH domain of any 2C3 antibody, variant, or derivative may be any suitable sequence, for instance any sequence as follows:

(SEQ ID NO: 23)
MVLSLLYLLTALPGILS;
or
(SEQ ID NO: 39)
MGSTAILGLLLAVLQGVCA

The leader sequence for the VL domain of any 2C3 antibody, variant, or derivative may be any suitable sequence, for instance any sequence as follows:

(SEQ ID NO: 42)
MRVPAQLLGLLLLWLPDTRC;
or
(SEQ ID NO: 43)
MDMRVPAQLLGLLLLWLPDTRC

In another particular embodiment, the inhibitor of the IL-25:IL-25R signalling pathway is or comprises the antibody “D9.2” or any antibody or binding molecule as defined in WO2010/116123, EP2417161B1, U.S. Pat. No. 8,586,037B2, U.S. Pat. No. 8,852,589, or any member of said patent family (herein incorporated by reference in its entirety), or a functional fragment or derivative thereof.

The inhibitor of the IL-25:IL-25R signalling pathway may be or may comprise an antibody, or fragment or derivative thereof, that comprises the CDRs of D9.2. The CDRs may be determined by any technique or method, or a combination of such methods. The CDRs may be determined by any method discussed herein.

The CDRs of D9.2, according to one method of determination, are as follows:

D9.2 VH CDR 1:
(SEQ ID NO: 49)
SYWMN
D9.2 VH CDR 2:
(SEQ ID NO: 50)
RIDPYDSEIQYNQKFKD
D9.2 VH CDR 3:
(SEQ ID NO: 51)
SGGFDWFAY
D9.2 VL CDR 1:
(SEQ ID NO: 52)
RASENINSNLA
D9.2 VL CDR 2:
(SEQ ID NO: 53)
DVTNLAD
D9.2 VL CDR 3:
(SEQ ID NO: 54)
QHFWRPPYT

The VH domain of D9.2 may have a sequence as follows:

(SEQ ID NO: 55)
FFLATPTCVHSQVQLQQPGAELVRPGASVKLSCKTSGYTFISYWMNWVK
QGGPEQGLEWIGRIDPYDSEIQYNQKFKDKAILTVDKSSSAAYMQLISL
TSEDSAVYYCARSGGFDWFAYWGQGTLVTVS.

The VL domain of D9.2 may have a sequence as follows:

(SEQ ID NO: 56)
MSVLTQVLALLLLWLTDARCDIQMTQSPASLSVSVGETVTITCRASENI
NSNLAWYQQKKGKSPQLLVYDVTNLADGVPSRFSGSGSGTQYSLKINSL
QSEDFGSYYCQHFWRPPYTFGGGTNLEIK.

Thus, in an embodiment, the inhibitor of the IL-25:IL-25R signalling pathway is capable of binding to IL-25R, and comprises:
an antibody VH domain, or functional fragment or derivative thereof, comprising:
   a VH CDR1 comprising the amino acid sequence shown in SEQ ID NO: 49;
   a VH CDR2 comprising the amino acid sequence shown in SEQ ID NO: 50; and
   a VH CDR3 comprising the amino acid sequence shown in SEQ ID NO: 51; and
an antibody a VL domain, or functional fragment or derivative thereof, comprising:
   a VL CDR1 comprising the amino acid sequence shown in SEQ ID NO: 52;
   a VL CDR2 comprising the amino acid sequence shown in SEQ ID NO: 53; and
   a VL CDR3 comprising the amino acid sequence shown in SEQ ID NO: 54.

Therefore, in an aspect of the invention, there is provided an anti-IL-25R antibody for use in a method of treating, preventing, or ameliorating cancer of the intestine, wherein the anti-IL-25R antibody comprises:
an antibody VH domain, or functional fragment or derivative thereof, comprising:
   a VH CDR1 comprising the amino acid sequence shown in SEQ ID NO: 49;
   a VH CDR2 comprising the amino acid sequence shown in SEQ ID NO: 50; and
   a VH CDR3 comprising the amino acid sequence shown in SEQ ID NO: 51; and
an antibody a VL domain, or functional fragment or derivative thereof, comprising:
   a VL CDR1 comprising the amino acid sequence shown in SEQ ID NO: 52;
   a VL CDR2 comprising the amino acid sequence shown in SEQ ID NO: 53; and
   a VL CDR3 comprising the amino acid sequence shown in SEQ ID NO: 54.

The above recited CDRs may comprise one, two, or three amino acid substitutions compared to the recited sequence, which may be conservative substitutions. The substitutions may be such that the properties of the inhibitor are not affected, or are not negatively affected. For instance, binding affinity may be retained or improved.

In some embodiments, the antibody VH domain comprising the amino acid sequence shown in SEQ ID NO: 55, optionally comprising at least one, from one to ten, from one to five, from one to three, or one, two, three, four, five, six, seven, eight, nine, or ten amino acid substitutions. The substitutions may be in residues that are not within a CDR. In some embodiments, said amino acid substitutions are conservative amino acid substitutions. The substitutions may be such that the properties of the inhibitor are not affected, or are not negatively affected. For instance, binding affinity may be retained or improved. In a particular embodiment, the domain comprises from one to three conservative amino acid substitutions.

In some embodiments, the antibody VL domain comprising the amino acid sequence shown in SEQ ID NO: 56, optionally comprising at least one, from one to ten, from one to five, from one to three, or one, two, three, four, five, six, seven, eight, nine, or ten amino acid substitutions. The substitutions may be in residues that are not within a CDR. In some embodiments, said amino acid substitutions are conservative amino acid substitutions. The substitutions may be such that the properties of the inhibitor are not affected, or are not negatively affected. For instance, binding affinity may be retained or improved. In a particular embodiment, the domain comprises from one to three conservative amino acid substitutions.

In an embodiment, the inhibitor of the IL-25:IL-25R signalling pathway is capable of binding to IL-25R, and comprises an antibody VH domain comprising the amino acid sequence shown in SEQ ID NO: 55, and an antibody VL domain comprising the amino acid sequence shown in SEQ ID NO: 56.

Thus, in a particular aspect of the invention, there is provided an anti-IL-25R antibody for use in a method of treating, preventing, or ameliorating cancer of the intestine, wherein the anti-IL-25R antibody comprises an antibody VH domain comprising the amino acid sequence shown in SEQ ID NO: 55, and an antibody VL domain comprising the amino acid sequence shown in SEQ ID NO: 56.

In an embodiment, the inhibitor of the IL-25:IL-25R signalling pathway is or comprises an antibody molecule which binds IL-25R and which comprises an antibody VH domain comprising a VH CDR3 with the amino acid sequence of SEQ ID NO: 51.

In an embodiment, the inhibitor of the IL-25:IL-25R signalling pathway is or comprises an antibody molecule that comprises a VH domain which comprises a VH CDR3 of SEQ ID NO: 51 together with a CDR1 of SEQ ID NO: 49 and a CDR2 of SEQ ID NO: 50. The VH domain may be paired with a VL domain, for example a VL domain with a CDR1 of SEQ ID NO: 52, a CDR2 of SEQ ID NO: 53 and a CDR3 of SEQ ID NO: 54. In some embodiments, a VH domain may be paired with a VL domain of SEQ ID NO: 55.

In an embodiment, the inhibitor of the IL-25:IL-25R signalling pathway is or comprises an antibody molecule that comprises a VH domain which comprises a VH CDR1 of SEQ ID NO: 49, a VH CDR2 of SEQ ID NO: 50 and a VH CDR3 of SEQ ID NO: 51 and a VL domain which comprises a VL CDR1 of SEQ ID NO: 52, a VL CDR2 of SEQ ID NO: 53 and a VL CDR3 of SEQ ID NO: 54.

The VH domain may further comprise human or non-human framework regions, for example the framework regions shown in SEQ ID NO: 55. In some embodiments, the antibody molecule may comprise the VH domain of SEQ ID NO: 55.

The VL domain may further comprise human or non-human framework regions, for example the framework region shown in SEQ ID NO: 56. In some embodiments, the antibody molecule may comprise the VL domain of SEQ ID NO: 56.

In a particular embodiment, the inhibitor of the IL-25:IL-25R signalling pathway is or comprises an antibody molecule that comprises the VH domain of SEQ ID NO: 55 and the VL domain of SEQ ID NO: 56.

In a particular embodiment, the inhibitor of the IL-25:IL-25R signalling pathway is or comprises an antibody molecule that specifically binds mouse and/or human IL-17BR (also known as IL-25R) and which comprises:
a VH domain comprising a CDR1 having the amino acid sequence of SEQ ID NO: 49, a CDR2 having the amino acid sequence of SEQ ID NO: 50, and a CDR3 having the amino acid sequence of SEQ ID NO: 51; and,
a VL domain comprising a CDR1 having the amino acid sequence of SEQ ID NO: 52, a CDR2 having the amino acid sequence of SEQ ID NO: 53, and a CDR3 having the amino acid sequence of SEQ ID NO: 54.

The VH domain may comprise a human framework region or SEQ ID NO: 55.

The antibody may be cross reactive with both human IL-17BR and mouse IL-17BR.

The VL domain may comprise a human framework region or SEQ ID NO: 56.

The D9.2 antibody may comprise a constant region, for instance an IgG1 or IgG4 constant region.

In another particular embodiment, the inhibitor is or comprises a humanised form of the antibody D9.2 or any antibody or binding molecule as defined in WO2020/115319 (herein incorporated by reference in its entirety), or a functional fragment or derivative thereof.

The inhibitor of the IL-25:IL-25R signalling pathway may be or may comprise an antibody, or fragment or derivative thereof, that comprises the CDRs of humanised

D9.2. The CDRs may be determined by any technique or method, or a combination of such methods. Such methods include use of the Kabat numbering system, Chothia numbering system, IMGT numbering system, and/or the extended definition. The CDRs may be determined by modelling software (such as AbM or WAM), or by structural or crystallographic techniques to determine contact residues.

The CDRs of humanised D9.2, according to one method of determination, may have any of the following sequences.

Humanised D9.2 VH CDR 1:
SEQ ID NO: 49
Humanised D9.2 VH CDR 2:
(SEQ ID NO: 57)
RIDPYDSEIQYX₁QKFX₂X₃ (X₁ is N or A, X₂ is K or Q,
and X₃ is D or G);
SEQ ID NO: 50
;
(SEQ ID NO: 58)
RIDPYDSEIQYNQKFKG;
(SEQ ID NO: 59)
RIDPYDSEIQYNQKFQD;
(SEQ ID NO: 60)
RIDPYDSEIQYNQKFQG;
(SEQ ID NO: 61)
RIDPYDSEIQYAQKFKD;
(SEQ ID NO: 62)
RIDPYDSEIQYAQKFKG;
(SEQ ID NO: 63)
RIDPYDSEIQYAQKFQD;
or
(SEQ ID NO: 64)
RIDPYDSEIQYAQKFQG
Humanised D9.2 VH CDR 3:
SEQ ID NO: 51
Humanised D9.2 VL CDR 1:
SEQ ID NO: 52
Humanised D9.2 VL CDR 2:
SEQ ID NO: 53
Humanised D9.2 VL CDR 3:
SEQ ID NO: 54
;
or
(SEQ ID NO: 65)
QHFWGPPYT

The VH domain of humanised D9.2 may have a sequence as follows:

(SEQ ID NO: 66)
EVQLVQSGAEVKKPGASVKVSCKTSGYTFISYWMNWVRQAPGQGLEWMG
RIDPYDSEIQYNQKFKDRVTLTRDTSISTAYMELSRLRSDDTAVYYCAT
SGGFDWFAYWGQGTLVTVSS;
(SEQ ID NO: 67)
X₁VQLVQSGAEVKKPGASVKVSCKX₂SGYTFISYWMNWVRQAPGQGLEWM
GRIDPYDSEIQYX₃QKFX₄X₅RVTX6TRDTSISTAYMELSRLRSDDTAVY
YCARSGGFDWFAYWGQGTLVTVSS (X₁ is Q or E, X₂ is A
or T, X₃ is A or N, X₄ is Q or K, X₅ is G or D
and X₆ is M or L);
(SEQ ID NO: 68)
QVQLVQSGAEVKKPGASVKVSCKASGYTFISYWMNWVRQAPGQGLEWMG
RIDPYDSEIQYNQKFKDRVTMTRDTSISTAYMELSRLRSDDTAVYYCAR
SGGFDWFAYWGQGTLVTVSS;
(SEQ ID NO: 69)
EVQLVQSGAEVKKPGASVKVSCKTSGYTFISYWMNWVRQAPGQGLEWMG
RIDPYDSEIQYNQKFKDRVTLTRDTSISTAYMELSRLRSDDTAVYYCAR
SGGFDWFAYWGQGTLVTVSS;
(SEQ ID NO: 70)
EVQLVQSGAEVKKPGASVKVSCKTSGYTFISYWMNWVRQAPGQGLEWMG
RIDPYDSEIQYAQKFQGRVTLTRDTSISTAYMELSRLRSDDTAVYYCAR
SGGFDWFAYWGQGTLVTVSS;
(SEQ ID NO: 80)
QVQLQQPGAELVRPGASVKLSCKTSGYTFISYWMNWVKQGPEQGLEWIG
RIDPYDSEIQYNQKFKDKAILTVDKSSSAAYMQLISLTSEDSAVYYCAR
SGGFDWFAYWGQGTLVTVSA;
(SEQ ID NO: 81)
EVQLVQSGAEVKKPGASVKVSCKTSGYTFISYWMNWVRQAPGQGLEWIG
RIDPYDSEIQYNQKFKDKAILTVDTSISTAYMELSRLRSDDTAVYYCAR
SGGFDWFAYWGQGTLVTVSS;
(SEQ ID NO: 82)
EVQLVQSGAEVKKPGASVKVSCKTSGYTFISYWMNWVRQAPGQGLEWIG
RIDPYDSEIQYNQKFKDRVTLTRDTSISTAYMELSRLRSDDTAVYYCAT
SGGFDWFAYWGQGTLVTVSS;
(SEQ ID NO: 83)
EVQLVQSGAEVKKPGASVKVSCKTSGYTFISYWMNWVRQAPGQGLEWMG
RIDPYDSEIQYNQKFKDKVTLTRDTSISTAYMELSRLRSDDTAVYYCAT
SGGFDWFAYWGQGTLVTVSS;
(SEQ ID NO: 84)
EVQLVQSGAEVKKPGASVKVSCKTSGYTFISYWMNWVRQAPGQGLEWMG
RIDPYDSEIQYNQKFKDRATLTRDTSISTAYMELSRLRSDDTAVYYCAT
SGGFDWFAYWGQGTLVTVSS;
(SEQ ID NO: 85)
EVQLVQSGAEVKKPGASVKVSCKTSGYTFISYWMNWVRQAPGQGLEWMG
RIDPYDSEIQYNQKFKDRVILTRDTSISTAYMELSRLRSDDTAVYYCAT
SGGFDWFAYWGQGTLVTVSS;
or
(SEQ ID NO: 86)
EVQLVQSGAEVKKPGASVKVSCKTSGYTFISYWMNWVRQAPGQGLEWMG
RIDPYDSEIQYNQKFKDRVTLTVDTSISTAYMELSRLRSDDTAVYYCAT
SGGFDWFAYWGQGTLVTVSS

The VL domain of humanised D9.2 may have a sequence as follows:

(SEQ ID NO: 71)
DIQMTQSPSSLSASVGDRVTITCRASENINSNLAWYQQKPGKAPKLLLY
DVTNLADGVPSRFSGSGSGTDYTLTISSLQPEDFATYYCQHFWRPPYTF
GGGTKVEIK;
(SEQ ID NO: 72)
DIQMTQSPSSLSASVGDRVTITCRASENINSNLAWYQQKPGKAPKLLLY
DVTNLADGVPSRFSGSGSGTDYTLTISSLQPEDFATYYCQHFWGPPYTF
GGGTKVEIK
(SEQ ID NO: 88)
DIQMTQSPSSLSASVGDRVTITCRASENINSNLAWYQQKPGKAPKLLVY
DVTNLADGVPSRFSGSGSGTQYTLTISSLQPEDFATYYCQHFWRPPYTF
GGGTKVEIK
(SEQ ID NO: 89)
DIQMTQSPSSLSASVGDRVTITCRASENINSNLAWYQQKPGKAPKLLVY
DVTNLADGVPSRFSGSGSGTDYTLTISSLQPEDFATYYCQHFWRPPYTF
GGGTKVEIK
(SEQ ID NO: 90)
DIQMTQSPSSLSASVGDRVTITCRASENINSNLAWYQQKPGKAPKLLLY
DVTNLADGVPSRFSGSGSGTQYTLTISSLQPEDFATYYCQHFWRPPYTF
GGGTKVEIK
(SEQ ID NO: 91)
DIQMTQSPSSLSASVGDRVTITCRTSENIYSNLAWYQQKPGKAPKLLLY
DATNLGDGVPSRFSGSGSGTDYTLTISSLQPEDFATYYCQHFWGPPYTF
GGGTKVEIK

The humanised D9.2 antibody may comprise a constant region, for instance an IgG1 or IgG4 constant region. The constant region may be a hinge stabilised IgG4 constant region according to the following sequence:

(SEQ ID NO: 73)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK

The heavy chain of the humanised D9.2 antibody may have the following sequence:

(SEQ ID NO: 74)
QVQLVQSGAEVKKPGASVKVSCKASGYTFISYWMNWVRQAPGQGLEWMG
RIDPYDSEIQYNQKFKDRVTMTRDTSISTAYMELSRLRSDDTAVYYCAR
SGGFDWFAYWGQGTLVTVSSASTKGPSVFPLAPCSRSTSESTAALGCLV
KDYFPEPVTVSWNSGALTSGVHTFPAVLQSSGLYSLSSVVTVPSSSLGT
KTYTCNVDHKPSNTKVDKRVESKYGPPCPPCPAPEFLGGPSVFLFPPKP
KDTLMISRTPEVTCVVVDVSQEDPEVQFNWYVDGVEVHNAKTKPREEQF
NSTYRVVSVLTVLHQDWLNGKEYKCKVSNKGLPSSIEKTISKAKGQPRE
PQVYTLPPSQEEMTKNQVSLTCLVKGFYPSDIAVEWESNGQPENNYKTT
PPVLDSDGSFFLYSRLTVDKSRWQEGNVFSCSVMHEALHNHYTQKSLSL
SLGK

Thus, in an embodiment, the inhibitor of the IL-25:IL-25R signalling pathway is capable of binding to IL-25R, and comprises
an antibody VH domain, or functional fragment or derivative thereof, comprising:
   a VH CDR1 comprising the amino acid sequence shown in SEQ ID NO: 49;
   a VH CDR2 comprising the amino acid sequence shown in any one of SEQ ID NOs: 50, 57, 58, 59, 60, 61, 62, 63, or 64; and
   a VH CDR3 comprising the amino acid sequence shown in SEQ ID NO: 51; and
an antibody a VL domain, or functional fragment or derivative thereof, comprising:
   a VL CDR1 comprising the amino acid sequence shown in SEQ ID NO: 52;
   a VL CDR2 comprising the amino acid sequence shown in SEQ ID NO: 53; and
   a VL CDR3 comprising the amino acid sequence shown in SEQ ID NO: 54 or SEQ ID NO: 65.

Therefore, in an aspect of the invention, there is provided an anti-IL-25R antibody for use in a method of treating, preventing, or ameliorating cancer of the intestine, wherein the anti-IL-25R antibody comprises:
an antibody VH domain, or functional fragment or derivative thereof, comprising:
   a VH CDR1 comprising the amino acid sequence shown in SEQ ID NO: 49;
   a VH CDR2 comprising the amino acid sequence shown in any one of SEQ ID NOs: 50, 57, 58, 59, 60, 61, 62, 63, or 64; and
   a VH CDR3 comprising the amino acid sequence shown in SEQ ID NO: 51; and
an antibody a VL domain, or functional fragment or derivative thereof, comprising:
   a VL CDR1 comprising the amino acid sequence shown in SEQ ID NO: 52;
   a VL CDR2 comprising the amino acid sequence shown in SEQ ID NO: 53; and
   a VL CDR3 comprising the amino acid sequence shown in SEQ ID NO: 54 or SEQ ID NO: 65.

The above recited CDRs may comprise one, two, or three amino acid substitutions compared to the recited sequence, which may be conservative substitutions. The substitutions may be such that the properties of the inhibitor are not affected, or are not negatively affected. For instance, binding affinity may be retained or improved.

In another embodiment, the inhibitor of the IL-25:IL-25R signalling pathway is capable of binding to IL-25R, and comprises

an antibody VH domain, or functional fragment or derivative thereof, comprising:
a VH CDR1 comprising the amino acid sequence shown in SEQ ID NO: 49;
a VH CDR2 comprising the amino acid sequence shown in SEQ ID NO: 50 or
SEQ ID NO: 64; and
a VH CDR3 comprising the amino acid sequence shown in SEQ ID NO: 51; and

an antibody a VL domain, or functional fragment or derivative thereof, comprising:
a VL CDR1 comprising the amino acid sequence shown in SEQ ID NO: 52;
a VL CDR2 comprising the amino acid sequence shown in SEQ ID NO: 53; and
a VL CDR3 comprising the amino acid sequence shown in SEQ ID NO: 65.

Therefore, in an aspect of the invention, there is provided an anti-IL-25R antibody for use in a method of treating, preventing, or ameliorating cancer of the intestine, wherein the anti-IL-25R antibody comprises:

an antibody VH domain, or functional fragment or derivative thereof, comprising:
a VH CDR1 comprising the amino acid sequence shown in SEQ ID NO: 49;
a VH CDR2 comprising the amino acid sequence shown in SEQ ID NO: 50 or
SEQ ID NO: 64; and
a VH CDR3 comprising the amino acid sequence shown in SEQ ID NO: 51; and

an antibody a VL domain, or functional fragment or derivative thereof, comprising:
a VL CDR1 comprising the amino acid sequence shown in SEQ ID NO: 52;
a VL CDR2 comprising the amino acid sequence shown in SEQ ID NO: 53; and

a VL CDR3 comprising the amino acid sequence shown in SEQ ID NO: 65.

The above recited CDRs may comprise one, two, or three amino acid substitutions compared to the recited sequence, which may be conservative substitutions. The substitutions may be such that the properties of the inhibitor are not affected, or are not negatively affected. For instance, binding affinity may be retained or improved.

In another embodiment, the inhibitor of the IL-25:IL-25R signalling pathway comprises an antibody VH domain comprising the amino acid sequence shown in any one of SEQ ID NOs: 66, 67, 68, 69, or 70, optionally comprising at least one, from one to ten, from one to five, from one to three, or one, two, three, four, five, six, seven, eight, nine, or ten amino acid substitutions. The substitutions may be in residues that are not within a CDR. In some embodiments, said amino acid substitutions are conservative amino acid substitutions. The substitutions may be such that the properties of the inhibitor are not affected, or are not negatively affected. For instance, binding affinity may be retained or improved. In a particular embodiment, the domain comprises from one to three conservative amino acid substitutions.

In another embodiment, the inhibitor of the IL-25:IL-25R signalling pathway comprises an antibody VH domain comprising the amino acid sequence shown in any one of SEQ ID NOs: 66, 67, 68, 69, 70, 80, 81, 82, 83, 84, 85, or 86, optionally comprising at least one, from one to ten, from one to five, from one to three, or one, two, three, four, five, six, seven, eight, nine, or ten amino acid substitutions. The substitutions may be in residues that are not within a CDR. In some embodiments, said amino acid substitutions are conservative amino acid substitutions. The substitutions may be such that the properties of the inhibitor are not affected, or are not negatively affected. For instance, binding affinity may be retained or improved. In a particular embodiment, the domain comprises from one to three conservative amino acid substitutions.

In another embodiment, the inhibitor of the IL-25:IL-25R signalling pathway comprises an antibody VL domain comprising the amino acid sequence shown in SEQ ID NO: 71 or SEQ ID NO: 72, optionally comprising at least one, from one to ten, from one to five, from one to three, or one, two, three, four, five, six, seven, eight, nine, or ten amino acid substitutions. The substitutions may be in residues that are not within a CDR. In some embodiments, said amino acid substitutions are conservative amino acid substitutions. The substitutions may be such that the properties of the inhibitor are not affected, or are not negatively affected. For instance, binding affinity may be retained or improved. In a particular embodiment, the domain comprises from one to three conservative amino acid substitutions.

In another embodiment, the inhibitor of the IL-25:IL-25R signalling pathway comprises an antibody VL domain comprising the amino acid sequence shown in any one of SEQ ID NOS: 71, 72, 87, 88, 89, 90, or 91, optionally comprising at least one, from one to ten, from one to five, from one to three, or one, two, three, four, five, six, seven, eight, nine, or ten amino acid substitutions. The substitutions may be in residues that are not within a CDR. In some embodiments, said amino acid substitutions are conservative amino acid substitutions. The substitutions may be such that the properties of the inhibitor are not affected, or are not negatively affected. For instance, binding affinity may be retained or improved. In a particular embodiment, the domain comprises from one to three conservative amino acid substitutions.

In a particular embodiment, the inhibitor of the IL-25:IL-25R signalling pathway is capable of binding to IL-25R, and comprises an antibody VH domain comprising the amino acid sequence shown in any one of SEQ ID NOs: 68, 69, or 70, and an antibody VL domain comprising the amino acid sequence shown in SEQ ID NO: 72.

Thus, in a particular aspect of the invention, there is provided an anti-IL-25R antibody for use in a method of treating, preventing, or ameliorating cancer of the intestine, wherein the anti-IL-25R antibody comprises an antibody VH domain comprising the amino acid sequence shown in any one of SEQ ID NOs: 68, 69, or 70, and an antibody VL domain comprising the amino acid sequence shown in SEQ ID NO: 72.

In another embodiment, the inhibitor of the IL-25:IL-25R signalling pathway is or comprises a humanised antibody molecule comprising a heavy chain variable domain and a light chain variable domain, wherein a) the heavy chain variable domain (VH) comprises a VHCDR1 of SEQ ID NO: 49, a VHCDR2 of SEQ ID NO: 57, and a VHCDR3 of SEQ ID NO: 51, and b) the light chain variable domain (VL domain) comprises a VLCDR1 of SEQ ID NO: 52, a VLCDR2 of SEQ ID NO: 53, and a VLCDR3 of SEQ ID NO: 65. The antibody molecule may specifically bind to interleukin-17 receptor B (IL17BR), preferably the extracellular domain of IL17BR. Optionally the antibody may bind to the interleukin-17 receptor with a binding affinity of at least 75% of the binding affinity of the murine antibody D9.2 to interleukin-17 receptor B, as measured by ELISA. The expression level of the antibody may be at least 40% of the expression level of the murine antibody D9.2 in mammalian cells.

In another embodiment, the inhibitor of the IL-25:IL-25R signalling pathway is or comprises a humanised form of the antibody D9.2 that comprises the VH domain of SEQ ID NO: 67, optionally with up to four additional framework substitutions. For example, the antibody may comprise a VH domain of SEQ ID NO: 68, SEQ ID NO: 69 or SEQ ID NO: 70, optionally with up to four additional framework substitutions. The antibody may comprise the VL domain of SEQ ID NO: 71, optionally with up to four additional framework substitutions. In some embodiments, the antibody molecule of may comprise the VH domain of SEQ ID NO: 68 and the VL domain of SEQ ID NO: 72.

The heavy chain variable domain (VH domain) of the humanised form of the antibody D9.2 may comprise SEQ ID NO: 67 wherein X₃ is N. The heavy chain variable domain (VH domain) may comprise SEQ ID NO: 67 wherein X₄is K. The heavy chain variable domain (VH domain) may comprise SEQ ID NO: 67 wherein X₅ is D. The heavy chain variable domain (VH domain) may comprise SEQ ID NO: 67 wherein X₁ is Q. The heavy chain variable domain (VH domain) may comprise SEQ ID NO: 67 wherein X₂ is A.

The heavy chain variable domain (VH domain) may comprise SEQ ID NO: 67 wherein X₆ is M.

The heavy chain variable domain (VH domain) of the humanised form of the antibody D9.2 may comprise SEQ ID NO: 67 wherein X, is E. . . . The heavy chain variable domain (VH domain) may comprise SEQ ID NO: 67 wherein X₂ is T. The heavy chain variable domain (VH domain) may comprise SEQ ID NO: 67 wherein X₆ is L.

The heavy chain variable domain (VH domain) of the humanised form of the antibody D9.2 may comprise SEQ ID NO: 67 wherein X₃ is A. The heavy chain variable domain (VH domain) may comprise SEQ ID NO: 67 wherein X₄ is Q. The heavy chain variable domain (VH domain) may comprise SEQ ID NO: 67 wherein X₅ is G. The heavy chain variable domain (VH domain) may comprise SEQ ID NO: 67 wherein X, is E. The heavy chain variable domain (VH domain) may comprise SEQ ID NO: 67 wherein X₂ is T. The heavy chain variable domain (VH domain) comprises SEQ ID NO: 67 wherein X₆ is L.

The VH domain of the humanised form of the antibody D9.2 may be fused to an antibody constant region comprising SEQ ID NO: 73. The humanised D9.2 may comprise an amino acid sequence of the VH domain and constant region which comprises SEQ ID NO: 74.

The humanised D9.2 antibody may further comprise a second heavy chain variable domain and a second light chain variable domain which form an antigen binding site for an antigen other than IL17BR.

The following sequences relate to the humanised D9.2 antibodies.

VH sequence HA (CDRs shaded) (Kabat positions 1, 24, 60, 64, 65, 69 and 94 underlined)
SEQ ID NO: 66

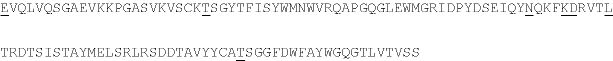

VH sequence (CDRs shaded) with modifications at Kabat positions 1, 24, 60, 64, 65 and 69
SEQ ID NO: 67

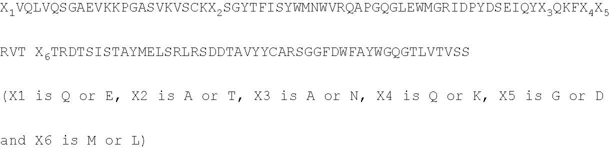

VH sequence HJ (CDRs shaded)
SEQ ID NO: 68

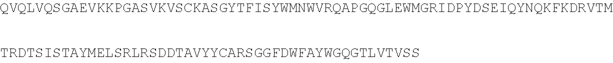

VH sequence HH (CDRs shaded)
SEQ ID NO: 69

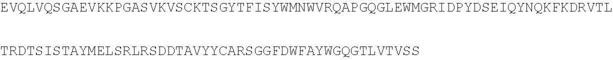

VH sequence HI (CDRs shaded)
SEQ ID NO: 70

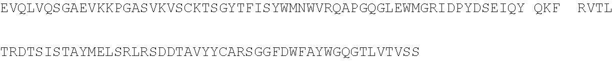

VL sequence KA (CDRs shaded) (Kabat position 93 underlined)
SEQ ID NO: 71

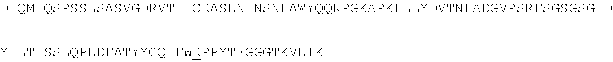

VL sequence KE (CDRs shaded)
SEQ ID NO: 72

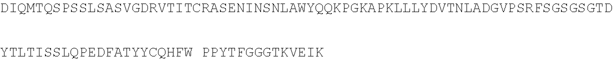

The following tables relate to the humanised D9.2 antibodies.

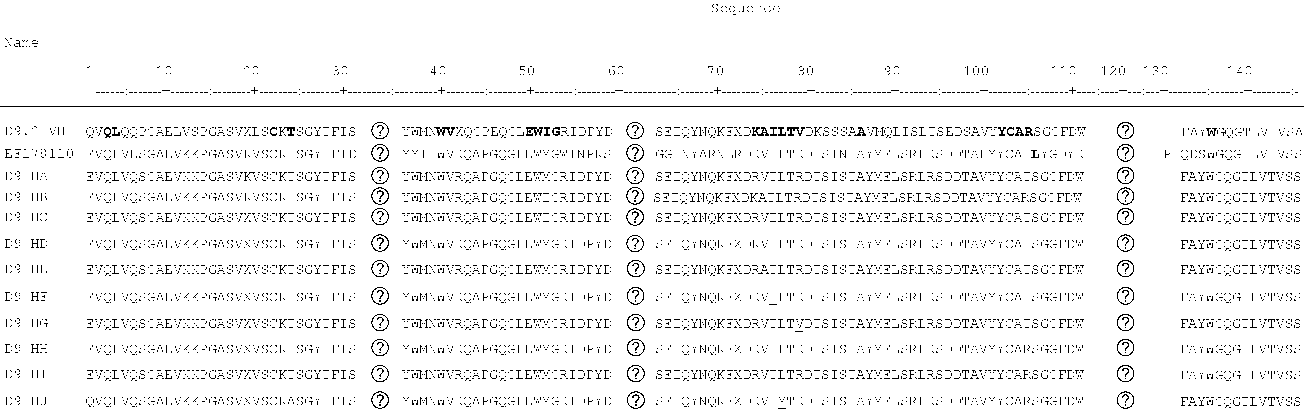

Dotted underline residues indicate back-translations to the Mouse Residue and solid underline residues indicate back-mutations to human germline

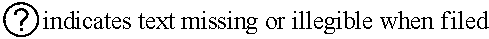

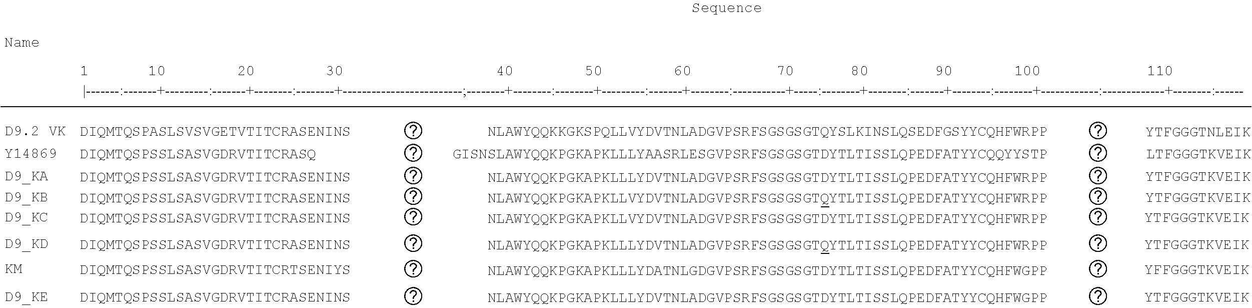

Dotted underline indicates back-translations to the Mouse Residue and the solid underline indicates the residue identified by molecular modelling.

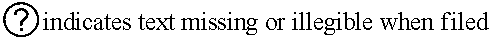

In the tables above, D9.2 VH is SEQ ID NO: 80, EF178110 is SEQ ID NO: 94. D9 HA is SEQ ID NO: 66, D9 HB is SEQ ID NO: 81, D9 HC is SEQ ID NO: 82, D9 HD is SEQ ID NO: 83, D9 HE is SEQ ID NO: 84, D9 HF is SEQ ID NO: 85, D9 HG is SEQ ID NO: 86, D9 HH is SEQ ID NO: 69, D9 HI is SEQ ID NO: 70, D9 HJ is SEQ ID NO: 68, D9.2 VK is SEQ ID NO: 87, Y14869 is SEQ ID NO: 95, D9_KA is SEQ ID NO: 71, D9_KB is SEQ ID NO: 88, D9_KC is SEQ ID NO: 89, D9_KD is SEQ ID NO: 90, KM is SEQ ID NO: 91, and D9_KE is SEQ ID NO: 72.

A CDR may be determined by technique or method, or a combination of such methods. Such methods include use of the Kabat numbering system, Chothia numbering system, the accumulation of both Kabat and Chothia, and/or the IMGT numbering system. The CDRs may be determined by modelling software (such as AbM, Accelrys, or WAM), or by structural or crystallographic techniques to determine contact residues. The CDRs may be determined by the contact definition which is based on observed antigen contacts and is described in MacCallum et al. (J. Mol. Biol., 262:732-745, 1996). The CDRs may be determined by the conformational definition, which identified residues that make enthalpic contributions to antigen binding (Makabe et al., Journal of Biological Chemistry, 283:1156-1166, 2008). Thus, while examples of CDRs are given herein, the inhibitor of the IL-25:IL-25R signalling pathway may comprise CDRs that are determined from any VH or VL sequence disclosed herein according to any suitable method.

As used herein, a conservative substitution is a replacement of an amino acid residue with an amino acid residue of a different type that has similar properties to the original residue. For instance, a conservative substitution may be one that does not change the functional properties of the polypeptide in which the original residue is found. In an example, a conservative substitution may be any replacement within the group: G, A, V, L, or I; within the group: S, C, T, M; within the group: F, Y, W; within the group: H, K, R; or within the group: D, E, N, Q.

In embodiments of the invention, the cancer of the intestine may be any cancer involving abnormal cell growth in the intestine of a subject. The cancer may be a primary tumour arising in the intestine. The cancer of the intestine may be secondary tumour, which has spread from a primary tumour in another tissue. The cancer of the intestine may be colorectal cancer (CRC). CRC may also be referred to as bowel cancer, colon cancer, or rectal cancer. CRC is caused by the abnormal growth of cells within the large intestine, for instance the abnormal growth of cells within the colon or rectum. CRC may encompass: colorectal adenocarcinomas, such as mucinous adenocarcinoma or signet ring cell adenocarcinoma; gastrointestinal carcinoid tumours; primary colorectal lymphomas; gastrointestinal stromal tumours;

leiomyosarcomas; and colitis-associated cancer (CAC). The CRC may be mismatch-repair-deficient (dMMR) CRC or mismatch-repair-proficient (pMMR) CRC. The CRC may be consensus molecular subtype (CMS) 1, CMS2, CMS3, and/or CMS4.

In some embodiments, the cancer of the intestine does not include CAC. In some embodiments, the cancer of the intestine is pMMR CRC. In a particular embodiment, the CRC comprises intestinal epithelial cells with mutations in the APC tumour suppressor gene.

In some embodiments, the disease to be treated is IL25high CRC. In some embodiments, the disease to be treated has a high frequency of ILC2s. In an embodiment, the tumour has an increased frequency of ILC2s compared to the ILC2 frequency in adjacent normal intestine. This may be determined by comparison of a tumour biopsy to non-tumour intestinal tissue from the same subject, or to the expected ILC2 frequency in non-tumour intestinal tissue. “Adjacent normal intestine” may mean intestinal tissue that does not comprise tumour cells or tumour stroma, and which is taken from a part of the intestine in which the tumour is situated.

In some embodiments, a tumour biopsy from the subject is tested before treatment. The tumour biopsy may be tested in order to determine if the tumour comprises mutations in the APC tumour suppressor gene. Alternatively, or in addition, the tumour biopsy may be tested in order to determine if the tumour is IL25high. Alternatively, or in addition, the tumour biopsy may be tested in order to determine if the tumour has a high frequency of ILC2s.

The inventors have noted that response to immune checkpoint inhibitor therapy in CRC patients is largely limited to mismatch-repair-deficient (dMMR) CRC, comprising only a small proportion (15%) of total CRC cases. By contrast, the vast majority of CRC, following the conventional pathway initiated by mutations in the APC gene, are mismatch-repair-proficient (pMMR), and rarely respond to immune checkpoint inhibitors. Furthermore, in the past decade, efforts to develop novel immunotherapies for CRC, such as tumour vaccines and adoptive T cell transfer, have shown little efficacy. Recently, the phase III clinical trial IMblaze370 using the anti-PD-L1 checkpoint inhibitor atezolizumab to treat CRC patients (>98% had mismatch-repair-proficient CRC) failed to meet the primary end point.

However, the inventors demonstrate herein that, when treating intestinal cancer, inhibitors of the IL-25:IL-25R signalling pathway shift the balance of the immune response away from MDSCs and towards infiltration by anti-tumoural CD8 T cell infiltration. As such, the blockade of the IL-25:IL-25R signalling pathway may increase the efficacy of immune checkpoint inhibitor therapy.

As such, in an embodiment, there is provided an inhibitor of the IL-25:IL-25R signalling pathway for use in a method of treating, preventing, or ameliorating cancer of the intestine, wherein the method further comprises immune checkpoint inhibitor therapy.

The inhibitor of the IL-25:IL-25R signalling pathway may be any disclosed herein. In a particular embodiment, there is provided an anti-IL-25 antibody, derivative, or functional fragment thereof for use in a method of treating, preventing, or ameliorating cancer of the intestine, wherein the method further comprises immune checkpoint inhibitor therapy. In another embodiment, there is provided there is provided an anti-IL-25R antibody, derivative, or functional fragment thereof for use in a method of treating, preventing, or ameliorating cancer of the intestine, wherein the method further comprises immune checkpoint inhibitor therapy.

The cancer of the intestine may be any disclosed herein. In a particular embodiment, the cancer of the intestine is pMMR CRC. The pMMR CRC may comprise intestinal epithelial cells with mutations in the APC tumour suppressor gene.

The immune checkpoint inhibitor therapy may be administered before, at the time of, or after the administration of the inhibitor of the IL-25:IL-25R signalling pathway. The immune checkpoint inhibitor may be physically separate from, or combined with, the inhibitor of the IL-25:IL-25R signalling pathway.

The immune checkpoint inhibitor therapy may comprise the administration of an inhibitor of the PD-1: PD-L1 pathway. The inhibitor of the PD-1: PD-L1 pathway may specifically bind to PD-1 or specifically bind to PD-L1. For instance, the inhibitor of the PD-1: PD-L1 pathway may be an anti-PD-1 antibody or an anti-PD-L1 antibody. Also encompassed are compositions comprising functional fragments or derivatives of such antibodies.

The immune checkpoint inhibitor therapy may comprise the administration of an inhibitor of the CTLA-4:CD80/CD86 pathway. The inhibitor of the CTLA-4:CD80/CD86 pathway may specifically bind to CTLA-4. For instance, the inhibitor of the CTLA-4:CD80/CD86 pathway may be an anti-CTLA-4 antibody. Also encompassed are compositions comprising functional fragments or derivatives of such antibodies.

The immune checkpoint inhibitor therapy may target LAG-3, TIM-3, TIGIT, VISTA, B7-H4, BTLA, and/or Siglec-15. Inhibitors targeting any of these molecules may specifically bind to said molecule, for instance the inhibitor may be an antibody, functional fragment, or derivative thereof, capable of specifically binding to any of said molecules.

Thus, in an embodiment, there is provided an inhibitor of the IL-25:IL-25R signalling pathway for use in a method of treating, preventing, or ameliorating cancer of the intestine, wherein the method further comprises an immune checkpoint inhibitor therapy targeted towards a molecule selected from the group: PD-1, PD-L1, CTLA-4, LAG-3, TIM-3, TIGIT, VISTA, B7-H4, BTLA, Siglec-15, and any combination thereof. In a particular embodiment, the immune checkpoint inhibitor therapy is a combination targeted towards CTLA-4 and PD-1/PD-L1.

In an aspect of the invention, there is provided a kit comprising an immune checkpoint inhibitor and an inhibitor of the IL-25:IL-25R signalling pathway. In another aspect of the invention, there is provided a pharmaceutical composition comprising an immune checkpoint inhibitor and an inhibitor of the IL-25:IL-25R signalling pathway.

In an embodiment, there is provided an inhibitor of the IL-25:IL-25R signalling pathway for use in a method of treating, preventing, or ameliorating cancer of the intestine, wherein the method does not comprise the administration of a HER inhibitor. In an embodiment, there is provided an inhibitor of the IL-25:IL-25R signalling pathway for use in a method of treating, preventing, or ameliorating cancer of the intestine, wherein the method does not comprise the administration of an EGFR (HER1) inhibitor. The HER inhibitor or EGFR inhibitor may be any as disclosed in WO2017/194554 (herein incorporated by reference).

In another aspect of the invention, there is provided a method of treating, preventing, or ameliorating cancer of the intestine in a subject in need thereof, comprising administering an effective amount of an inhibitor of the IL-25:IL-25R signalling pathway. The inhibitor of the IL-25:IL-25R signalling pathway any be any such inhibitor, including any disclosed herein. The cancer of the intestine may be any cancer of the intestine, including any disclosed herein. The subject of the treatment may be any subject, including any disclosed herein.

Also provided is a method of reducing ILC2s, M-MDSCs, and/or G-MDSCs in a tumour in the intestine, comprising administering an effective amount of an inhibitor of the IL-25:IL-25R signalling pathway. The inhibitor of the IL-25:IL-25R signalling pathway any be any such inhibitor, including any disclosed herein. The tumour of the intestine may be the result of any cancer of the intestine, including any disclosed herein. The subject of the treatment may be any subject, including any disclosed herein. The method may result in the treatment, prevention, or amelioration of the cancer.

Also provided is a method of altering the immune composition in the microenvironment of in a tumour in the intestine, comprising administering an effective amount of an inhibitor of the IL-25:IL-25R signalling pathway. The immune microenvironment may be altered to reduce ILC2s, M-MDSCs, and/or G-MDSCs and to increase ILC1s and/or CD8 T cells. The inhibitor of the IL-25:IL-25R signalling pathway any be any such inhibitor, including any disclosed herein. The tumour of the intestine may be the result of any cancer of the intestine, including any disclosed herein. The subject of the treatment may be any subject, including any disclosed herein. The method may result in the treatment, prevention, or amelioration of the cancer.

In another aspect of the invention, there is provided use of an inhibitor of the IL-25:IL-25R signalling pathway for the manufacture of a medicament for the treatment, prevention, or amelioration of cancer of the intestine.

As used herein, a therapeutically effective amount is the amount sufficient to effect any one or more beneficial or desired results.

As used herein, ameliorating means a lessening or improvement in one or more symptoms as compared to not administering the inhibitor of the IL-25:IL-25R signalling pathway.

In an embodiment, the subject of the treatments disclosed herein is an animal subject, for instance a jawed-vertebrate, mammal, or domestic animal. Hence, the inhibitor of the IL-25:IL-25R signalling pathway may be used to treat any mammal, for example livestock (for example, a horse), pets, or may be used in other veterinary applications. In a particular embodiment, the subject is a human. In some embodiments, the subject has been diagnosed with cancer of the intestine.

The inhibitors disclosed herein may be present as part of a pharmaceutical composition. The pharmaceutical composition may further comprise a pharmaceutically acceptable vehicle, a pharmaceutically acceptable carrier, a pharmaceutically acceptable excipient, a pharmaceutically acceptable stabilizer, or a pharmaceutically acceptable preservative, or any combination thereof.

To be pharmaceutically acceptable, a substance or combination of substances must be suitable for the formulation of pharmaceutical compositions or a medicament.

The pharmaceutical compositions of the invention may be manufactured, in a further aspect, using a process comprising contacting a therapeutically effective amount of any of inhibitors disclosed herein and a pharmaceutically acceptable vehicle, a pharmaceutically acceptable carrier, a pharmaceutically acceptable excipient, a pharmaceutically acceptable stabilizer, or pharmaceutically acceptable preservative, or any combination thereof.

A composition comprising any one of inhibitors disclosed herein may take any number of forms known in the art depending on the manner in which it is to be used. For example, in the form of a liquid, powder, suspension, tablet, capsule, ointment, cream, gel, hydrogel, aerosol, spray, micellar solution, transdermal patch, or any other suitable form for storage, shipping, or administration.

In some embodiments, the composition may be in a format suitable for storage or shipping. For instance, as part of lyophilized compositions which may include other components if necessary for lyophilization or storage, such as stabilizers. Alternatively, the composition may be in solution with a diluent, and may be present at a concentration appropriate for storage or shipping.

In some embodiments, the composition may be in a format suitable for administration to a patient. For instance, in the form of a liquid suitable for injection, or in a concentrated form suitable for administration after a preparatory step. The composition may be in association with carriers for administration, and may be sterile. The composition may be in a format suitable for injection into the patient, for instance intravenous injection or subcutaneous injection. The composition may be in a format suitable for topical administration to the intestine, rectum, or bowel.

In some embodiments, the composition may be present in slow-release compositions.

It will be appreciated that the amount of the composition that is required is determined by its biological activity and bioavailability, which in turn depends on the mode of administration, the physiochemical properties of the composition, and whether it is being used as a monotherapy or in a combined therapy. The frequency of administration will also be influenced by the half-life of the agent within the subject to be treated. Optimal dosages to be administered may be determined by those skilled in the art and will vary depending on the context. Additional factors may depend on the particular subject being treated, the advancement of the disease, disorder, or condition, subject age, subject weight, subject sex, subject diet, and time of administration.

In some examples, the composition may be administered as a single dose, yearly dose, monthly dose, weekly dose, or daily dose. The dose may be administered two or more times a day. In an example, the dose may be from 0.001 μg to 10 mg per kg of body weight, 0.01 μg to 1 mg per kg of body weight, 0.05 μg to 100 μg per kg of body weight, or 0.1 μg to 10 μg per kg of body weight. In an example, the dose may be 0.07 μg to 700 mg, 0.7 μg to 70 mg, 3.5 μg to 7 mg, or 7 μg to 0.7 mg.

It is understood that wherever embodiments are described herein with the language “comprising,” otherwise analogous embodiments described in terms of “consisting of” and/or “consisting essentially of” are also provided.

Unless otherwise defined, all technical and scientific terms used herein have the same meaning as commonly understood by one of ordinary skill in the art to which this invention belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular.

All of the features described herein (including any accompanying claims, abstract and drawings), and/or all of the steps of any method or process so disclosed, may be combined with any of the above aspects in any combination, except combinations where at least some of such features and/or steps are mutually exclusive. Methods and materials similar to or equivalent to those described herein can be used in the practice and testing of the invention, and suitable methods and materials are described below.

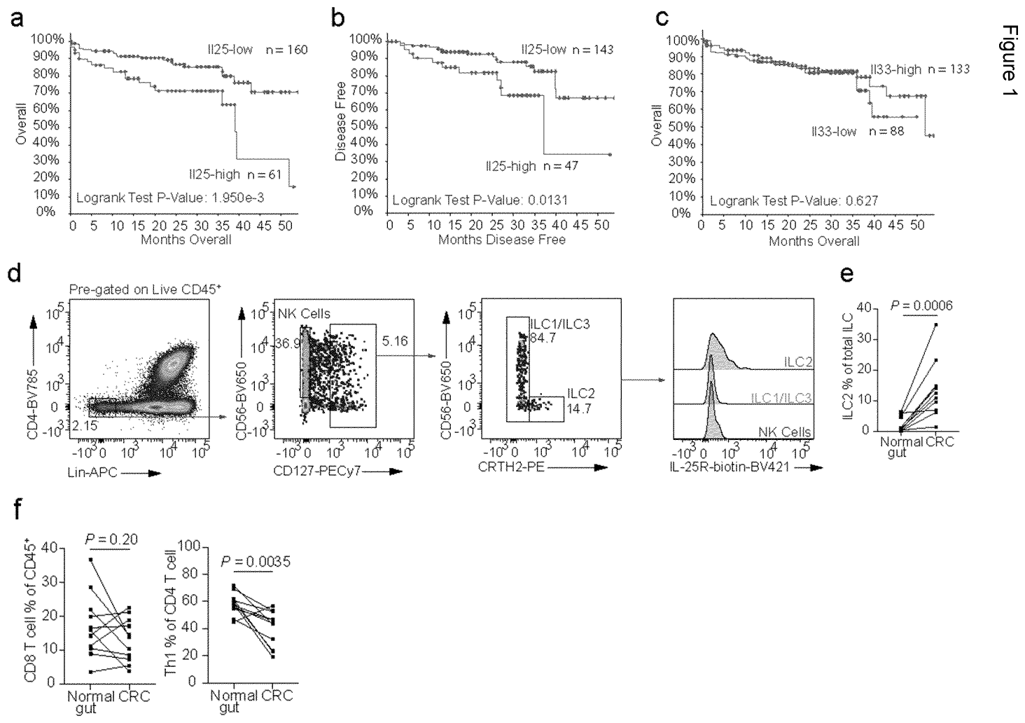

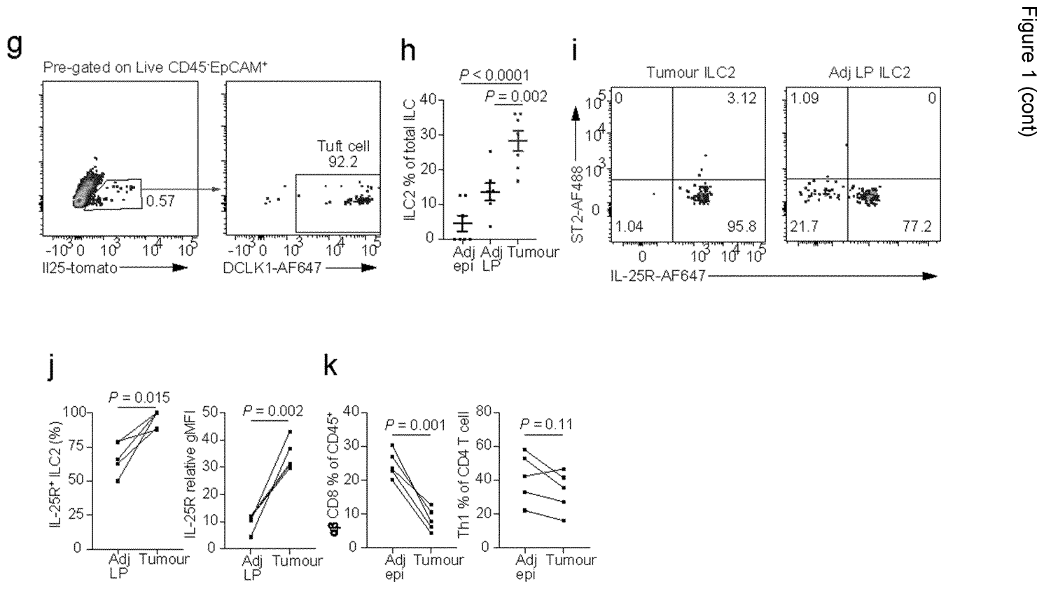

FIG. 1: IL-25R expressing ILC2s infiltrate human and Apc^{1322T/+} mouse model of colorectal cancer. a, b, Overall (a) and disease-free (b) survival of CRC patients from the TCGA Firehose Legacy microarray dataset, stratified into two groups by tumour Il25 expression. Il25-high and Il25-low groups are defined as patients with CRC that had tumour Il25 expression above and below the mean of the total samples, respectively. c, Overall survival of CRC patients from the TCGA Firehose Legacy microarray dataset, stratified into two groups by tumour Il33 expression. Il33-high and Il33-low groups are defined as patients with CRC that had tumour Il33 expression above and below the mean of the total samples, respectively. d, Gating strategy and IL-25R expression on human ILCs in primary patient CRC samples. e-f, Frequency of ILC2s (e), CD8 and Th1 T cells (f) in paired CRC and adjacent normal tissue from unselected patients with CRC. g, Representative FACS plots showing DCLK1⁺ tuft cell frequency within Il25-tomato expressing CD45⁻EpCAM⁺ epithelial cells in Apc^{1322T/+} mice. h, Frequency of ILC2s in paired tumours, adjacent normal epithelium (Adj epi) and lamina propria (Adj LP) in Apc^{1322T/+} mice (n=7). i, IL-25R and ST2 expression in mouse ILC2s from Apc^{1322T/+} mouse tumours and Adj LP. j, Percentage expression and expression level of IL-25R on ILC2s from paired tumours and Adj LP from Apc^{1322T/+} mice (n=5). Relative gMFI, geometric mean fluorescent intensity relative to isotype control. k, Frequency of ab CD8 T cells and Th1 cells in paired tumour and Adj epi from Apc^{1322T/+} mice (n=5). e, f, Each pair of dots indicates an individual human CRC patient; n=11. h, j, k, Data collected from female mice between 77-83 days of age; each dot or pair of dots indicates an individual mouse. Data pooled from two or more independent experiments and error bars show mean±SEM. Statistical significance determined by log-rank test (a-c), paired two-tailed t-test (e, f, j, k), one-way ANOVA with Tukey's post hoc (h).

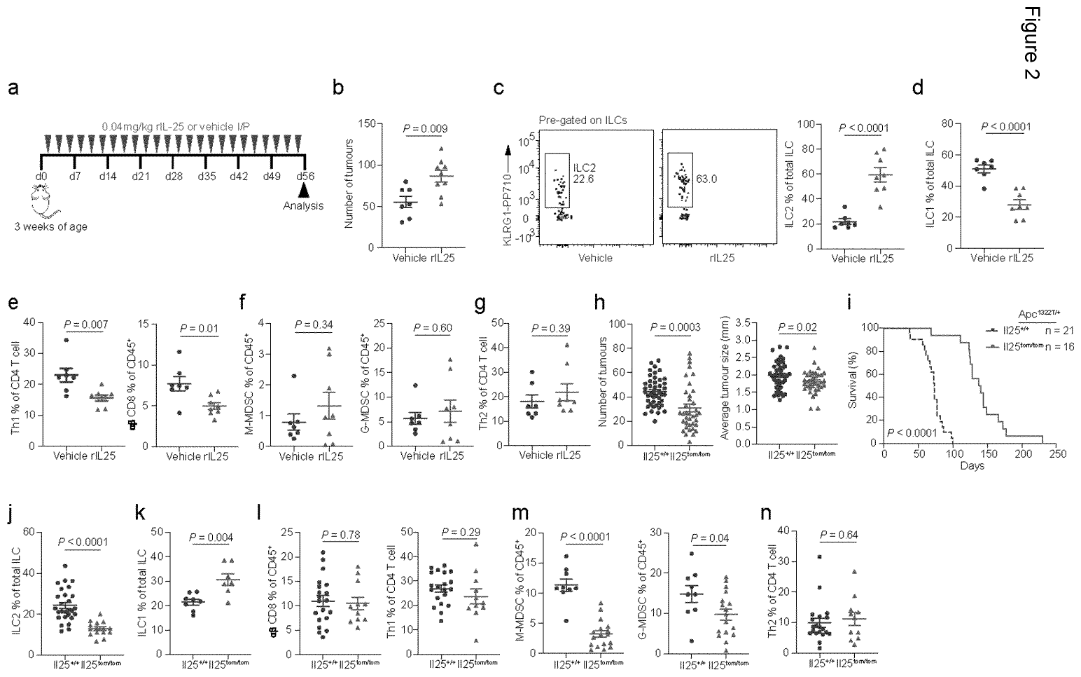

FIG. 2: IL-25 promotes intestinal tumours. a, Schematic of rIL-25 or vehicle treatment in Apc^{1322T/+} mice. b, Number of tumours in vehicle and rIL-25-treated Apc^{1322T/+} mice (vehicle, n=7; rIL25, n=9). c, Representative FACS plot and tumour ILC2 frequency in vehicle and rIL-25-treated Apc^{1322T/+} mice. d-g, Frequency of tumour infiltrating Tbet⁺ ILC1s (d), Th1 and ab CD8 T cells (e), M-MDSCs and G-MDSCs (f), and Th2 T cells (g), in vehicle and rIL-25-treated Apc^{1322T/+} mice (vehicle, n=7; rIL25, n=8). h, i, Number of tumours and average tumour size (Il25^{+/+}, n=44; Il25^{tom/tom}, n=37) (h) and survival (i) of Il25^{+/+} and Il25^{tom/tom} Apc^{1322T/+} mice. j-n, Frequency of tumour ILC2s (Il25^{+/+}, n=27; Il25^{tom/tom}, n=15) (j), ILC1s (Il25^{+/+}, n=8; Il25^{tom/tom}, n=7) (k), ab CD8 and Th1 T cells (Il25^{+/+}, n=20; Il25^{tom/tom}, n=11) (l), M-MDSCs and G-MDSCs (Il25^{+/+}, n=9; Il25^{tom/tom}, n=17) (m), and Th2 T cells (Il25^{+/+}, n=20; Il25^{tom/tom}, n=11) (n), from Il25^{+/+} and Il25^{tom/tom} Apc^{1322T/+} mice. b-h, j-n, Data collected from female mice between 77 to 83 days of age; each dot indicates an individual mouse. Data pooled from two or more independent experiments and error bars show mean±SEM. Statistical significance determined by unpaired two-tailed t-test, except in (i) calculated by two-sided log-rank test.

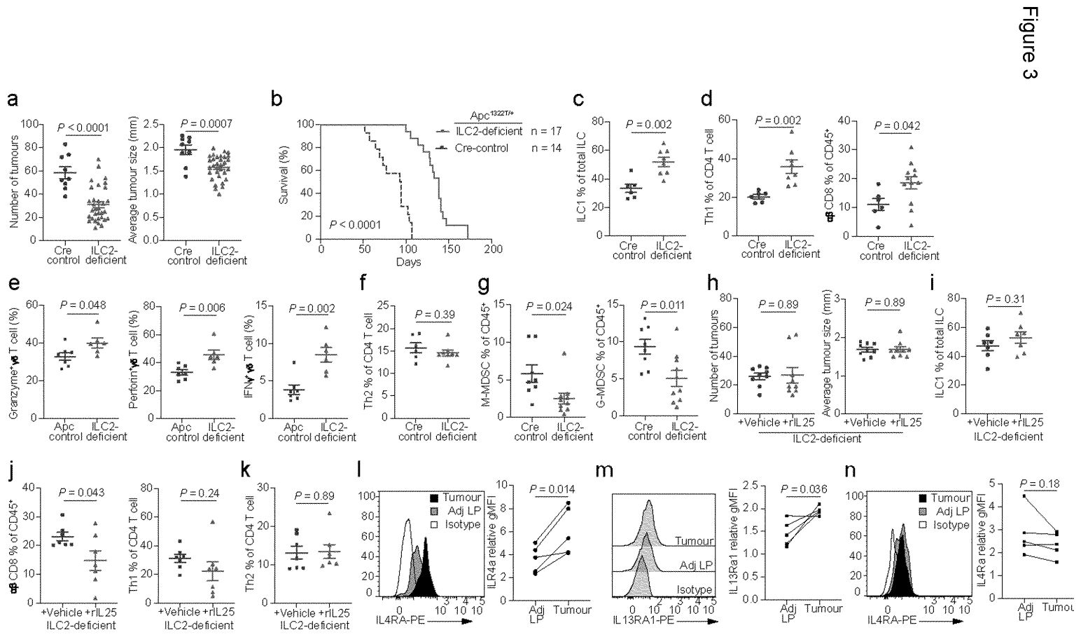

FIG. 3: ILC2s promote intestinal tumours through inhibiting anti-tumour immunity. a, b, Number of tumours and average tumour size (a) and survival (b) of ILC2-deficient (Rora^{f/f}Il7^{Cre/+}) and Cre control (Rora^{+/+}Il7r^{Cre/+}) Apc^{1322T/+} mice (Cre control, n=9; ILC2-deficient, n=32). c, d, Frequency of tumour ILC1s (Cre control, n=6; ILC2-deficient, n=8) (c), Th1 (Cre control, n=6; ILC2-deficient, n=8) and ab CD8 T cells (Cre control, n=6; ILC2-deficient, n=12) (d), in ILC2-deficient and Cre control Apc^{1322T/+} mice. e, Granzyme, perforin and IFNγ expression in tumour γδ T cells from ILC2-deficient and control (Rora^{+/+}Il7r^{+/+}) Apc^{1322T/+} mice (control, n=7; ILC2-deficient, n=6). f, g, Frequency of tumour Th2 T cells (Cre control, n=6; ILC2-deficient, n=8) (f) and M-MDSCs and G-MDSCs (Cre control, n=8; ILC2-deficient, n=10 (g) in ILC2-deficient and Cre control Apc^{1322T/+} mice. h, Number of tumours and average tumour size in vehicle or rIL-25-treated ILC2-deficient (Rora^{f/f}Il7^{Cre/+}) Apc^{1322T/+} mice (vehicle, n=9; rIL25, n=9). i-k, Frequency of tumour ILC1s (i), ab CD8 and Th1 T cells (j), and Th2 T cells (k), in vehicle or rIL-25-treated ILC2-deficient Apc^{1322T/+} mice (vehicle, n=7; rIL25, n=7). l, m, Representative FACS plots and expression level (relative gMFI) of IL4RA (l) and IL13RA1 (m), on M-MDSCs in tumour and adjacent lamina propria (Adj LP) from Apc^{1322T/+} mice (n=5). Relative gMFI, geometric mean fluorescent intensity relative to isotype control. n, Representative FACS plot and expression level (relative gMFI) of IL4RA on G-MDSCs from tumour and Adj LP of Apc^{1322T/+} mice (n=5). a, c-n, Data collected from female mice between 77 to 83 days of age; each dot indicates an individual mouse. Data pooled from two or more independent experiments and error bars show mean±SEM. Statistical significance determined by unpaired two-tailed t-test (a, c-k), two-sided log-rank test (b), and paired two-tailed t-test (l-n).

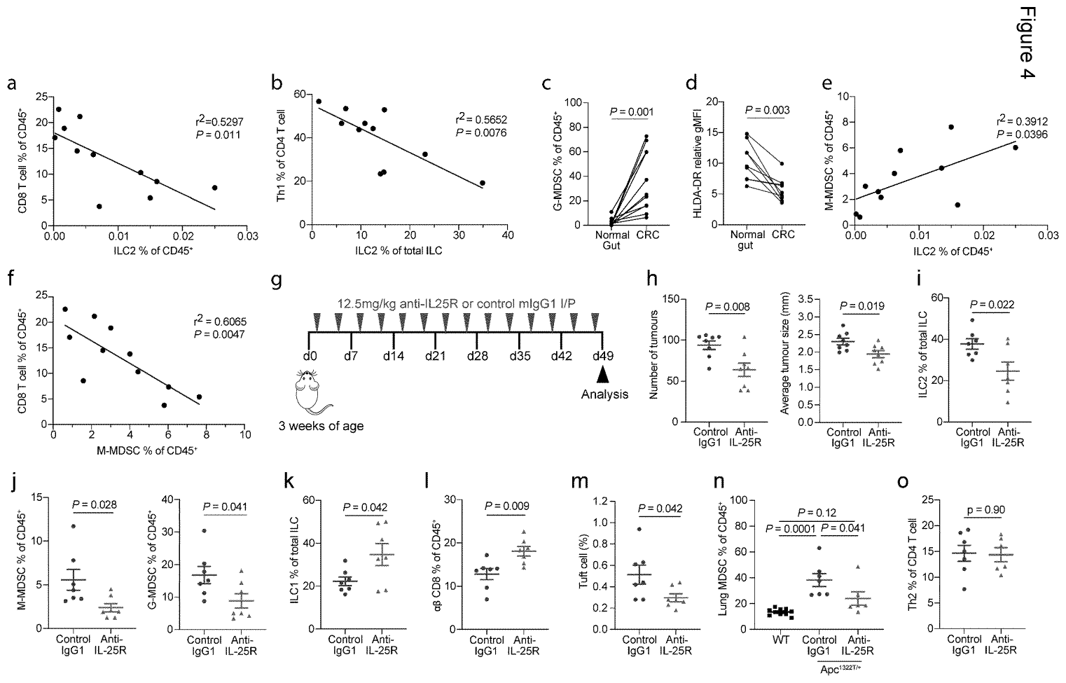

FIG. 4: Therapeutic pathway conservation in human CRC. a, Correlation of CD8 T cells and ILC2s in unselected human CRC samples. b, Correlation of Th1 T cells and ILC2s in unselected human CRC samples. c, Frequency of G-MDSCs in human CRC and adjacent normal gut. d, HLA-DR expression level (relative gMFI) of M-MDSCs in human CRC and adjacent normal gut. Relative gMFI, geometric mean fluorescent intensity relative to FMO control. FMO, full-minus-one. e, Correlation of M-MDSCs and ILC2s in unselected human CRC samples. f, Correlation of M-MDSC and CD8 T cells in unselected human CRC samples. g, Schematic of anti-IL25R or vehicle treatment in Apc^{1322T/+} mice. h, Number of tumours and average tumour size in anti-IL25R or control IgG1-treated Apc^{1322T/+} mice (control IgG1, n=8; anti-IL-25R, n=8). i-m, Frequency of ILC2 (i), M-MDSCs and G-MDSCs (j), ILC1s (k), αβ CD8 T cells (l), and tuft cells (m), in anti-IL25R or control IgG1-treated Apc^{1322T/+} mice (control IgG1, n=7; anti-IL-25R, n=7). n, Frequency of total CD11b⁺Gr1⁺ MDSCs in lung from wild-type non-tumour-bearing C57BL/6 Apc^{+/+} mice (WT) and anti-IL25R or control IgG1-treated Apc^{1322T/+} mice (WT, n=10; control IgG1, n=7; anti-IL-25R, n=6). o, Frequency of Th2 T cells in anti-IL25R or control IgG1-treated Apc^{1322T/+} mice (control IgG1, n=7; anti-IL-25R, n=6). a-f, Each dot or pair of dots represents an individual human CRC patient; (a, b, c, e, f, n=11; d, n=9). h-o, Data collected from age-matched female mice treated with anti-IL25R or control IgG1, and pooled from two or more independent experiments; error bars show mean±SEM. Each dot indicates an individual mouse. Statistical significance determined by Pearson's rank correlation coefficient (a, b, e, f), paired two-tailed t-test (c, d), unpaired two-tailed t test (h-m, o) and one-way ANOVA with Tukey's post hoc (n).

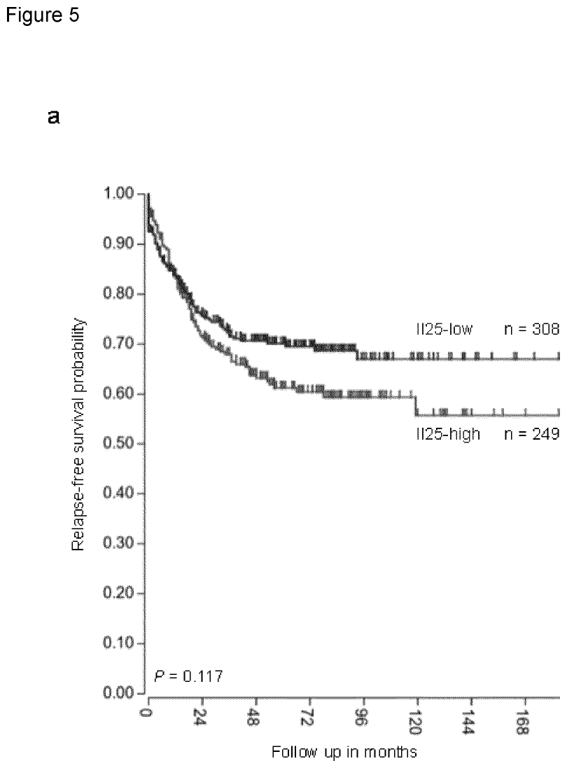

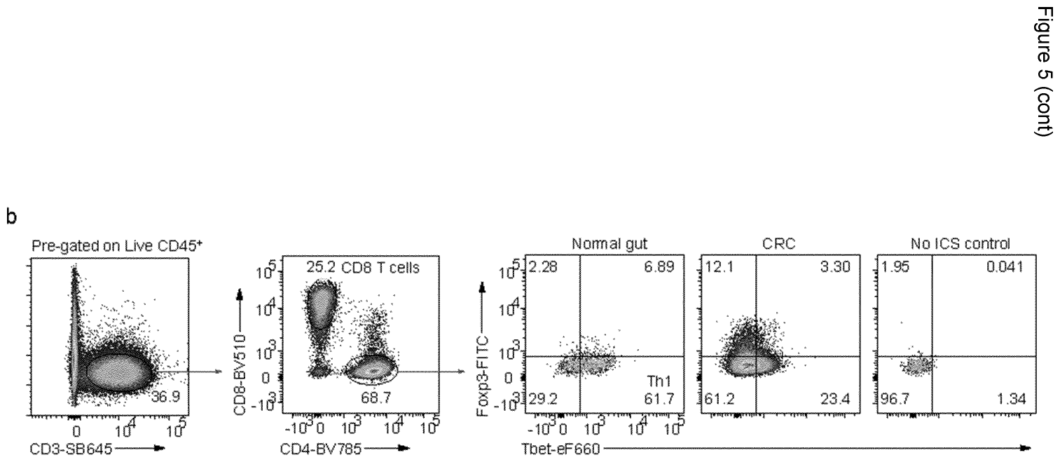

FIG. 5: Human CRC tumour characterisation. a, Graph shows relapse-free survival of CRC patients from the large publicly available RNA dataset by Marisa et al. 2013 (Gene Expression Classification of Colon Cancer into Molecular 454 Subtypes: Characterization, Validation, and Prognostic Value. PLOS Medicine 10, 455 e1001453, doi: 10.1371/journal.pmed.1001453 (2013)), analysed through the R2 platform, and stratified into two groups by tumour Il25 expression. Il25-high and Il25-low groups are defined as patients with CRC that had tumour Il25 expression above and below the mean expression respectively. Statistical significance determined by log-rank test. b, Gating strategy of CD8 T cells and Th1 T cells in human CRC and adjacent normal gut. SB645, Super Bright 645 fluorochrome.

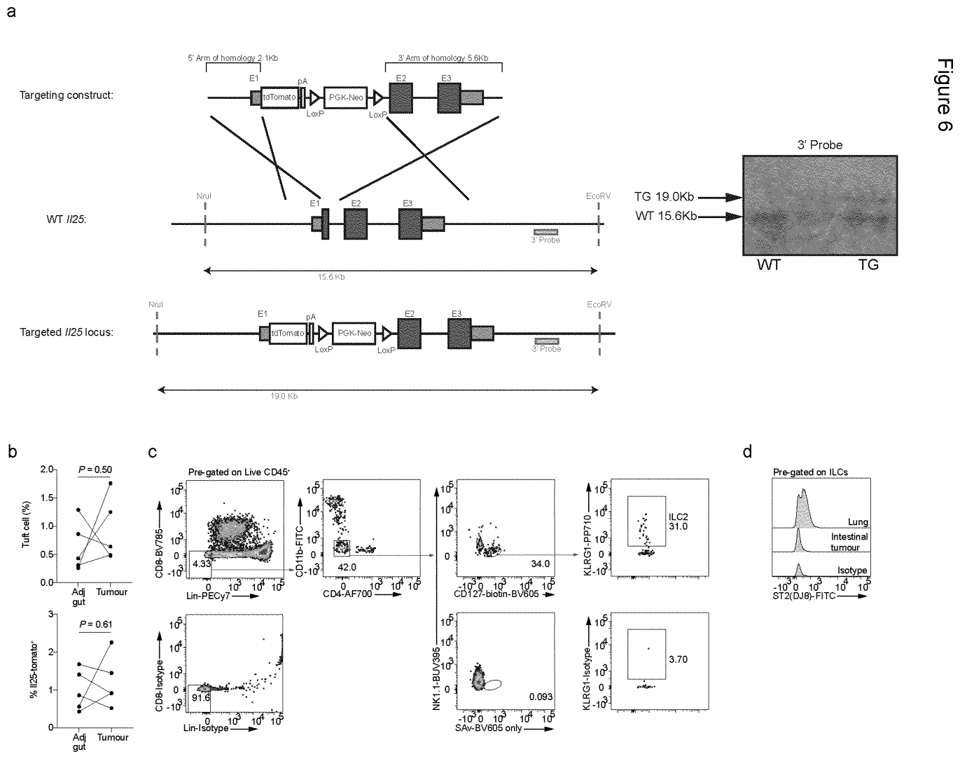

FIG. 6: IL-25 and immune cells in mouse Apc^{1322T/+} tumour model. a, Schematic of Il25-tomato construct and Southern analysis. b, Tuft cell frequency (top) and Il25-tomato expression (bottom) in EpCAM⁺ cells in tumour and adjacent gut from Il25^{tom/+}Apc^{1322T/+} mice (n=5). c, Gating strategy of ILC2s in Apc^{1322T/+} mice. d, Plot shows ST2 (clone DJ8) expression on total ILCs in intestinal tumours and lung from Apc^{1322T/+} mice. e, Gating strategy of γδ, αβ CD4 and αβ CD8 T cells in Apc^{1322T/+} mice. f, Frequency of γδ T cells in tumour and adjacent epithelium (Adj epi) in Apc^{1322T/+} mice (n=5). g, Granzyme, perforin and IFNγ expression in ab CD8 T cells in tumour and Adj epi from Apc^{1322T/+} mice (n=8). h, Granzyme, perforin and IFNγ expression in γδ T cells in tumour and Adj epi from Apc^{1322T/+} mice (n=6). i, j, Gating strategy (i) and frequency (j) of M-MDSCs and G-MDSCs in tumour, Adj epi, and adjacent lamina propria (Adj LP) from Apc^{1322T/+} mice (n=7). b, f-h, j, Data collected from female mice between 77 to 83 days of age; each dot or pair of dots indicate an individual mouse. Data pooled from two or more independent experiments and error bars show mean±SEM. Statistical significance determined by paired two-tailed t-test (b, f, g, h), and one-way ANOVA with Tukey's post hoc (j).

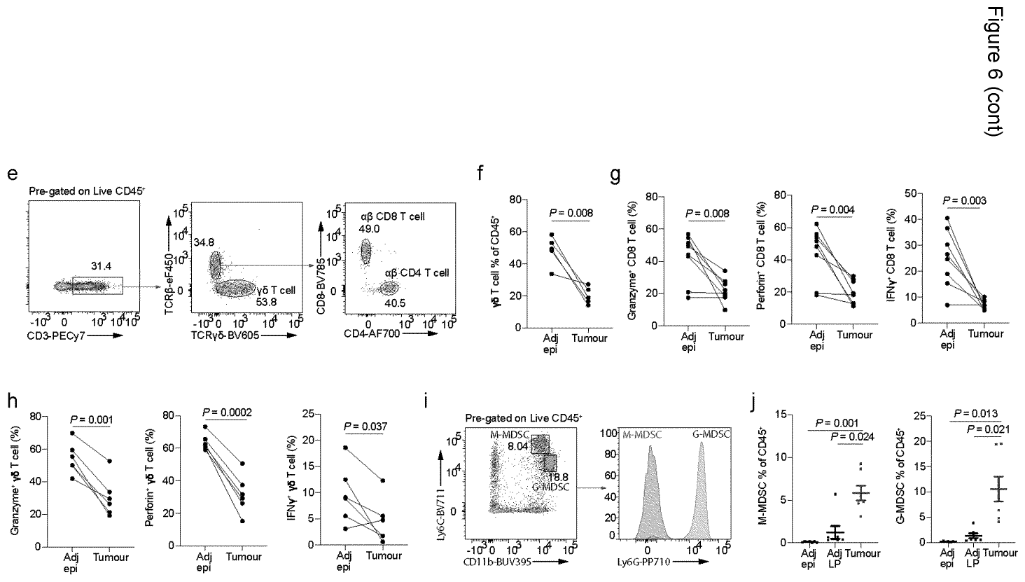

FIG. 7: IL-25 promotes tumours in Apc^{1322T/+} mice. a-c, Gut length and number of tumour normalised to length (a) and average tumour size (b) (vehicle, n=7; rIL25, n=9), and frequency of Ki67⁺ EpCAM⁺ tumour cells (c) (vehicle, n=7; rIL25, n=8), in vehicle and rIL-25 treated Apc^{1322T/+} mice. d, Gating strategy of ILC1s in Apc^{1322T/+} mouse tumours. e, Frequency of ILC2s in mesenteric lymph nodes (MLN) of vehicle and rIL-25 treated Apc^{1322T/+} mice (vehicle, n=7; rIL25, n=8). f, Frequency of tuft cells in tumours of vehicle and rIL-25 treated Apc^{1322T/+} mice (vehicle, n=7; rIL25, n=8). g-i, Gating strategy and frequency of macrophages and conventional dendritic cells (cDC) (g), plasmacytoid dendritic cells (pDC) (h), and mast cells and basophils (i), in vehicle and rIL-25 treated Apc^{1322T/+} mice (vehicle, n=7; rIL25, n=8). j, Frequency of tumour γδ T cells in vehicle and rIL-25 treated Apc^{1322T/+} mice (vehicle, n=7; rIL25, n=8). k, Gating strategy and frequency of tumour Th1, Th2 and Tregs in vehicle and rIL-25 treated Apc^{1322T/+} mice (vehicle, n=7; rIL25, n=8). Data collected from female mice between 77 to 83 days of age; each dot indicates an individual mouse. Data pooled from two or more independent experiments and error bars show mean±SEM. Statistical significance determined by unpaired two-tailed t-test.

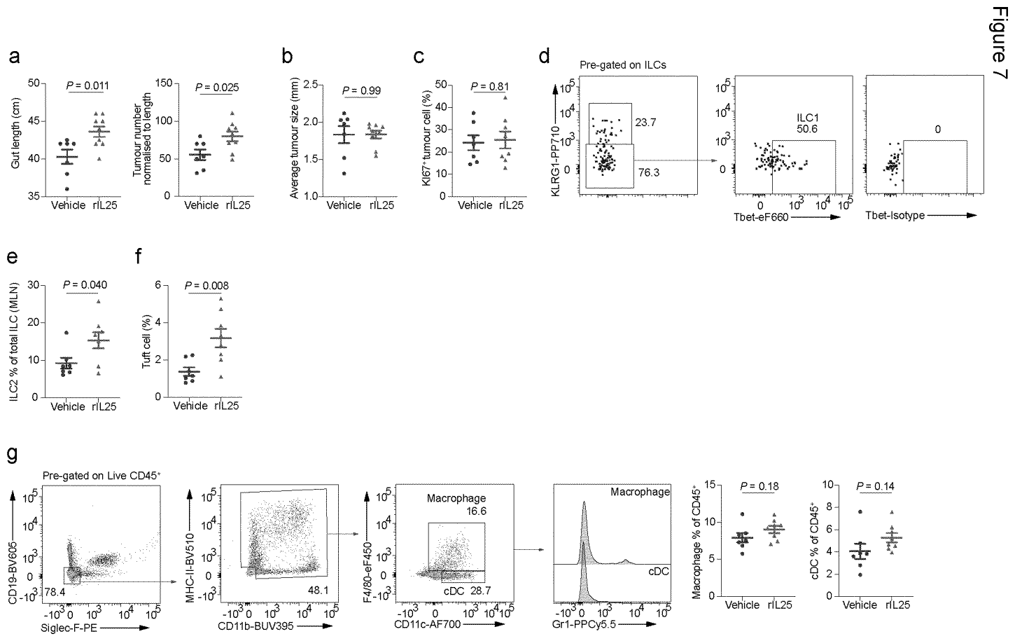

FIG. 8: Deletion of IL-25 protects from intestinal tumours. a, Survival of Il25^{+/+} and Il25^{tom/tom} Apc^{1322T/+} male breeders. b, Frequency of ILC2s in mesenteric lymph nodes (MLN) of Il25^{+/+} and Il25^{tom/tom} Apc^{1322T/+} mice (Il25^{+/+}, n=10; Il25^{tom/tom}, n=13). c-h, Frequency of tumour macrophages (c) and conventional dendritic cells (cDC) (d) (Il25^{+/+}, n=8; Il25^{tom/tom}, n=9), plasmacytoid dendritic cells (pDC) (e) and mast cells and basophils (f) (Il25^{+/+}, n=15; Il25^{tom/tom}, n=10), γδ T cells (g) and Tregs (h) (Il25^{+/+}, n=20; Il25^{tom/tom}, n=11), in Il25^{+/+} and Il25^{tom/tom} Apc^{1322T/+} mice. b-h, Data collected from female mice between 77 to 83 days of age; each dot indicates an individual mouse. Data pooled from two or more independent experiments and error bars show mean±SEM. Statistical significance determined by two-sided log-rank test (a), and unpaired two-tailed t-test (b-h).

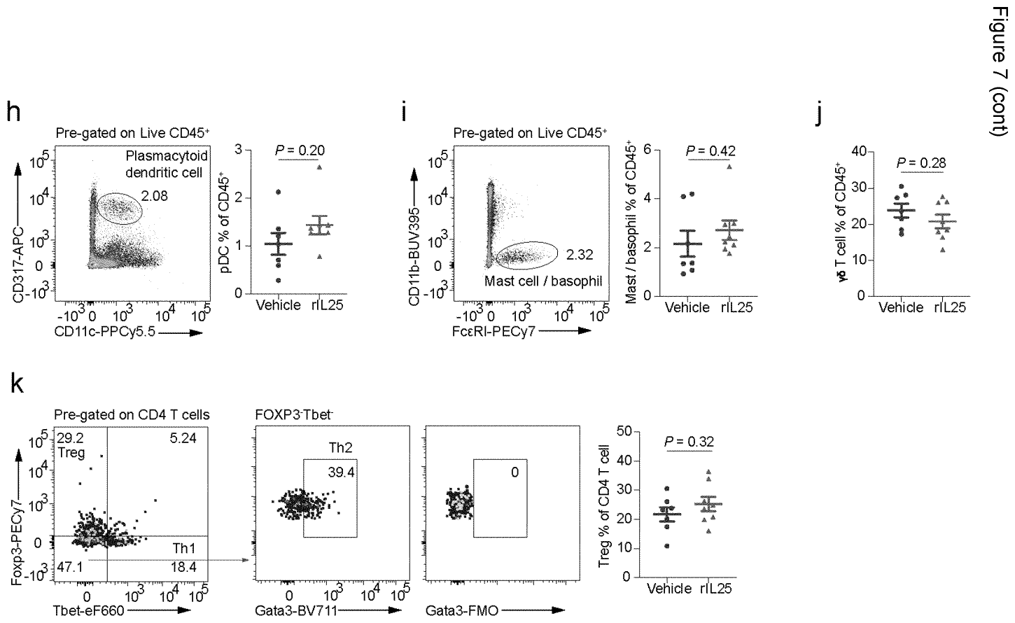

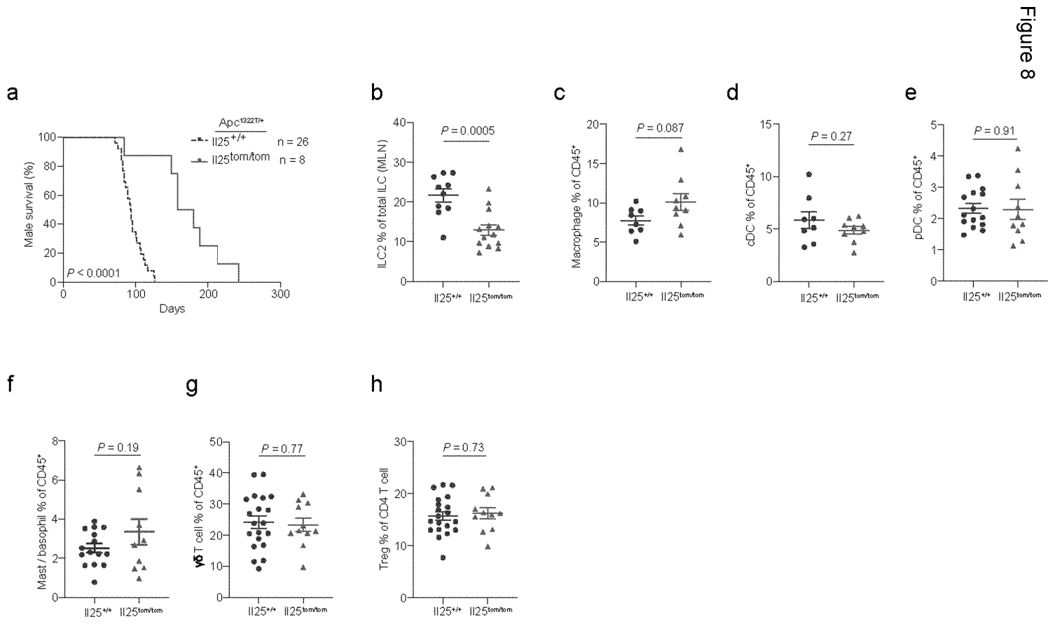

FIG. 9: Ablation of ILC2s protects from intestinal tumours. a, Representative FACS plot showing ILC2s in control ILC2-replete (Rora^{+/+}Il7r^{Cre/+}) and ILC2-deficient (Rora^{f/f}Il7^{Cre/+}) Apc^{1322T/+} mice. b, Survival of ILC2-deficient and control Apc^{1322T/+} male breeders. c-g, Frequency of tumour γδ T cells (c), macrophages (d), conventional dendritic cells (CDC) (e), plasmacytoid dendritic cells (pDC) (f), mast cells and basophils (g), in ILC2-deficient and control Apc^{1322T/+} mice (Cre control, n=6; ILC2-deficient, n=12). h, Frequency of tumour Tregs in ILC2-deficient and control Apc^{1322T/+} mice (Cre control, n=6; ILC2-deficient, n=8). c-h, Data collected from female mice between 77 to 83 days of age; each dot indicates an individual mouse. Data pooled from two or more independent experiments and error bars show mean±SEM. Statistical significance determined by two-sided log rank test (b), and unpaired two-tailed t-test (c-h).

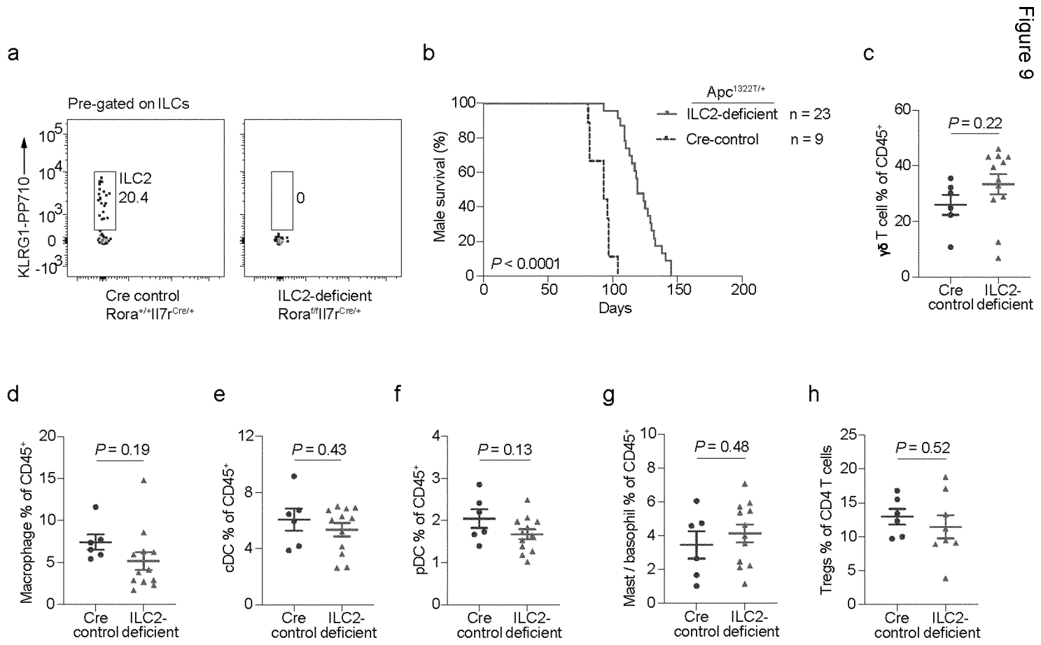

FIG. 10: IL-25 does not induce tumours in ILC2-deficient mice. a, Gut length in vehicle or rIL-25 treated ILC2-deficient (Rora^{f/f}Il7r^{Cre/+}) Apc^{1322T/+} mice (vehicle, n=9; rIL25, n=9). b, Representative FACS plot showing lack-of ILC2s in vehicle and rIL-25 treated ILC2-deficient Apc^{1322T/+} mice. c-j, Frequency of tumour M-MDSCs and G-MDSCs (c), macrophages (d), conventional dendritic cells (CDC) (e), plasmacytoid dendritic cells (pDC) (f), mast cells and basophils (g), Tregs (h), γδ T cells (i) and tuft cells (j), in vehicle and rIL-25 treated ILC2-deficient Apc^{1322T/+} mice (vehicle, n=7; rIL25, n=7). Data collected from female mice between 77 to 83 days of age; each dot indicates an individual mouse. Data pooled from two or more independent experiments and error bars show mean±SEM. Statistical significance determined by unpaired two-tailed t-test.

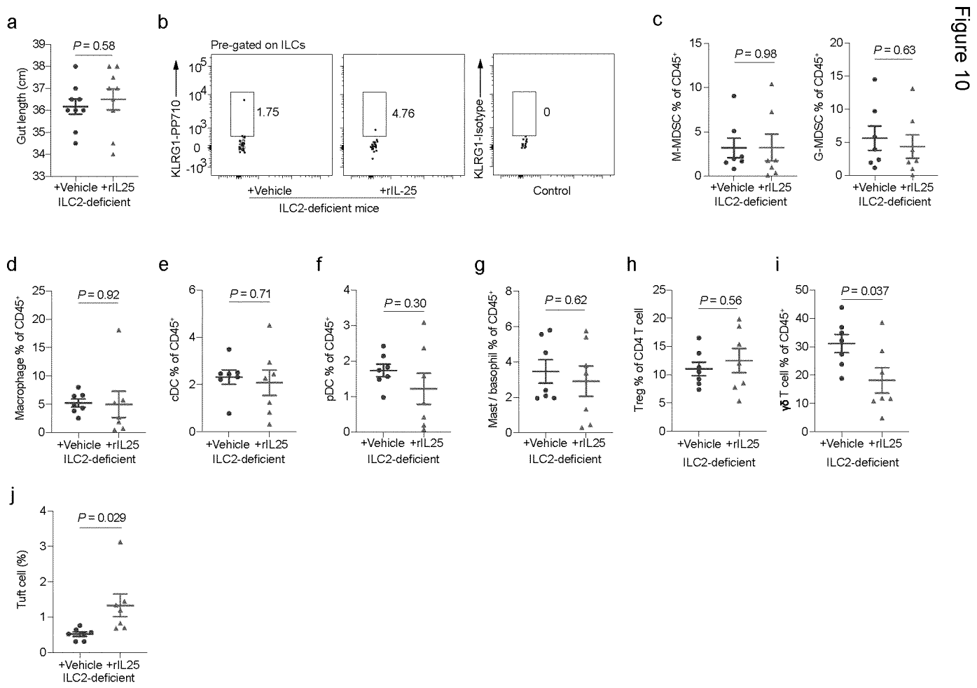

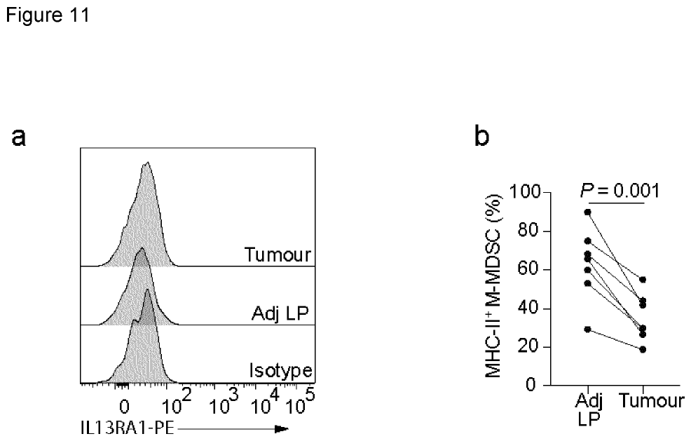

FIG. 11: Receptor expression on MDSCs. a, Representative FACS plot showing IL13RA1 receptor staining on G-MDSCs from Apc^{1322T/+} mice. b, Frequency of MHC-II expression on tumour and adjacent lamina propria (Adj LP) M-MDSCs in Apc^{1322T/+} mice (n=7). Relative gMFI, geometric mean fluorescent intensity relative to isotype control. Data collected from female mice between 77 to 83 days of age; each dot indicates an individual mouse. Data pooled from two or more independent experiments. Statistical significance determined by paired two tailed t-test.

FIG. 12: Characterisation of MDSCs in human CRC samples. a, Gating strategy of M-MDSCs and G-MDSCs in human CRC. b, Representative FACS plots showing expression of CD86, PD-L1, HVEM, CD155 and CD112 on M-MDSCs and G-MDSCs from human CRC. c, Representative FACS plots showing expression of PD-1, CTLA-4 and LAG-3 on CD8 T cells from human CRC. d, e, Quantification of CTLA-4, PD-1, LAG-3 (d), and Tbet (e) expression on CD8 T cells in human CRC. Relative gMFI, geometric mean fluorescent intensity relative to isotype control. d, e, Each pair of dots represents an individual human CRC patient; n=9. Statistical significance determined by paired two-tailed t-test.

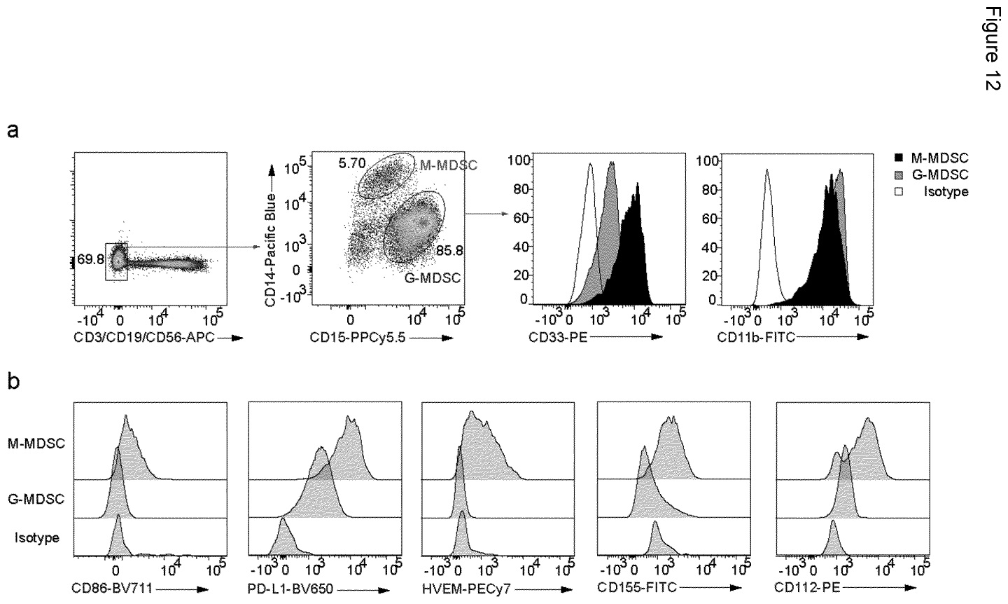

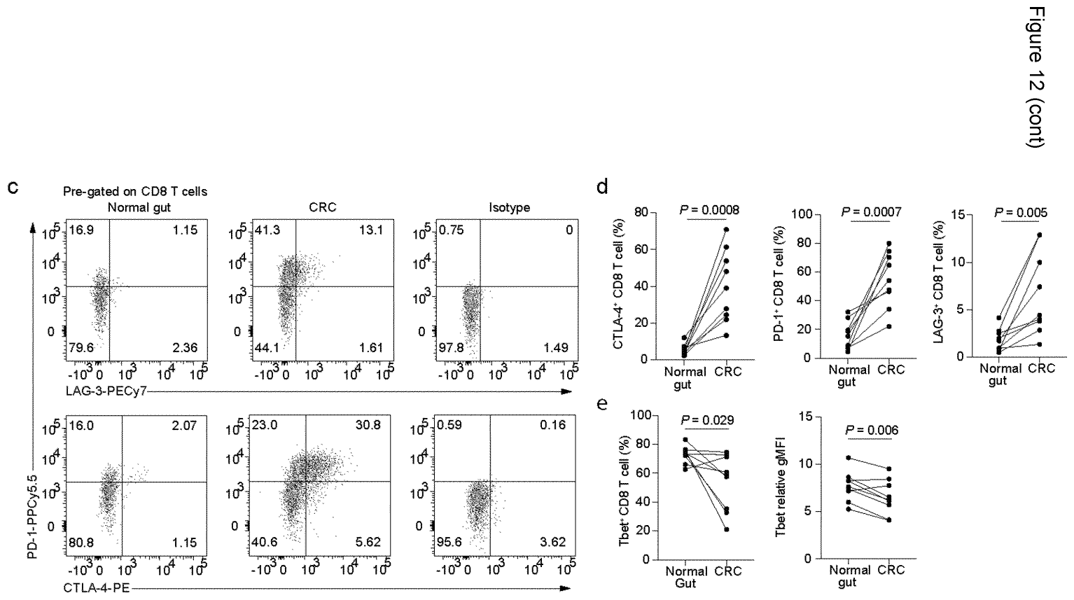

FIG. 13: Characterisation of immune cells in tumours following anti-IL25R treatment. a, Frequency of ILC2s in mesenteric lymph nodes (MLN) of anti-IL25R or control IgG1-treated Apc^{1322T/+} mice (control IgG1, n=7; anti-IL-25R, n=7). b, Representative FACS plots show gating of total lung MDSCs in wild-type non-tumour bearing Apc^{+/+} mice and anti-IL25R or vehicle treated Apc^{1322T/+} mice. C i, Frequency of tumour Th1 T cells (control IgG1, n=7; anti-IL-25R, n=7) (c), macrophages (control IgG1, n=7; anti-IL-25R, n=6) (d), conventional dendritic cells (CDC) (control IgG1, n=7; anti-IL-25R, n=6) (e), plasmacytoid dendritic cells (pDC) (control IgG1, n=7; anti-IL-25R, n=6) (f), mast cells and basophils (control IgG1, n=7; anti-IL-25R, n=6) (g), γδ T cells (control IgG1, n=7; anti-IL-25R, n=7) (h), and Tregs (control IgG1, n=7; anti-IL-25R, n=6) (i), in anti-IL25R or control IgG1-treated Apc^{1322T/+} mice. Data collected from age-matched female mice treated with anti-IL25R or control IgG1, and pooled from two or more independent experiments; error bars show mean±SEM. Each dot indicates an individual mouse. Statistical significance determined by unpaired two-tailed t-test.

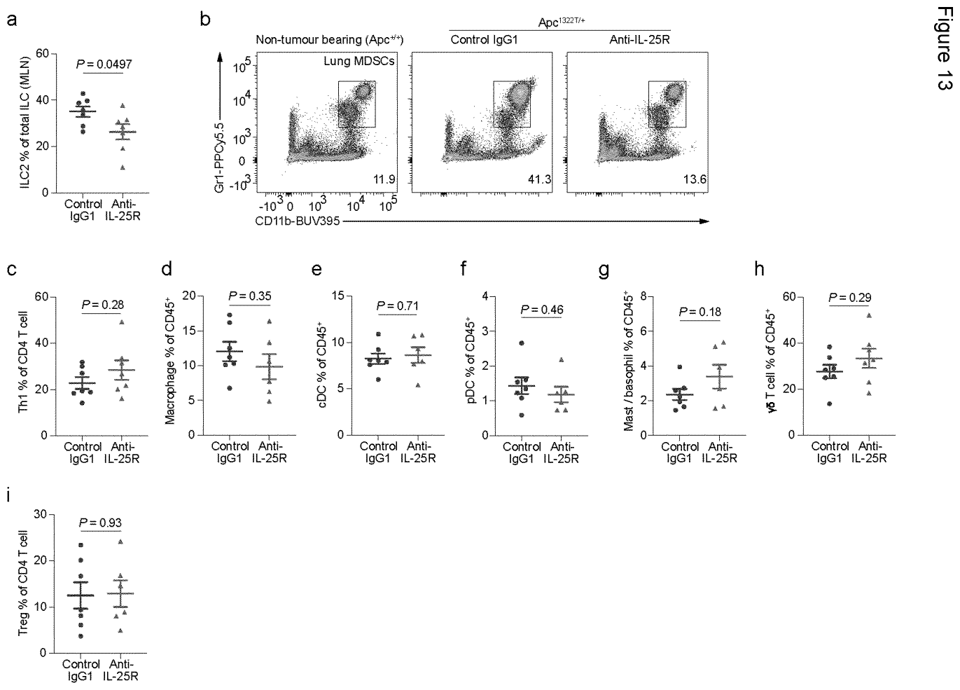

FIG. 14: TCGA data of IL25 expression across different CMS subtypes. a, IL25 expression in CRC tumours segregated by consensus molecular subtypes (CMS) 1, CMS2, CMS3, and CMS4, analysed from the online publicly available TCGA database.

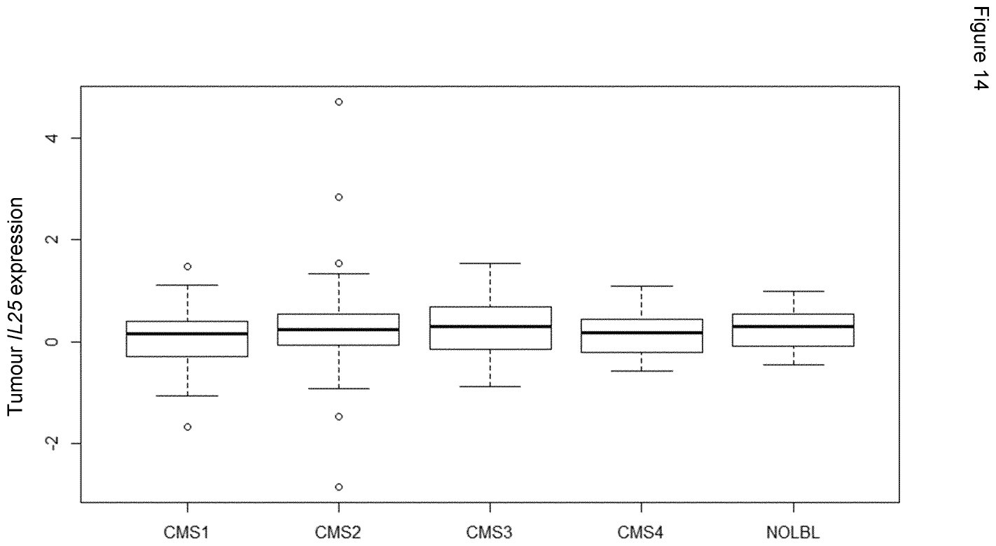

FIG. 15: Therapeutic intervention blocking the IL-25-ILC2 axis promotes anti-tumour type 1 immunity and decreases tumour burden a, Schematic of anti-IL25R or vehicle treatment starting in adult (7-weeks of age) Apc^{1322T/+} mice. b, Number of tumours and average tumour size in anti-IL25R or control IgG1-treated Apc^{1322T/+} mice (control IgG1, n=7; anti-IL-25R, n=7). c,d, Frequency (c), and IL-4 and IL-13 expression (d) in tumour ILC2s in anti-IL25R or control IgG1-treated Apc^{1322T/+} mice (control IgG1, n=7; anti-IL-25R, n=7). e, Argi expression in tumour M-MDSCs in anti-IL25R or control IgG1-treated Apc^{1322T/+} mice (control IgG1, n=7; anti-IL-25R, n=7). f-h, Frequency and IFNγ expression in tumour αβ CD8 T cells (f), γδ T cells (g), and This (h), in anti-IL25R or control IgG1-treated Apc^{1322T/+} mice (control IgG1, n=7; anti-IL-25R, n=7). i, Frequency of tuft cells in anti-IL25R or control IgG1-treated Apc^{1322T/+} mice (control IgG1, n=7; anti-IL-25R, n=7). Data collected from female mice at 11-weeks of age, treated with anti-IL25R or control IgG1 (treatment started in adults at 7-weeks of age), and pooled from two or more independent experiments; error bars show mean±SEM. Each dot indicates an individual mouse. Statistical significance determined by unpaired two-tailed t-test.

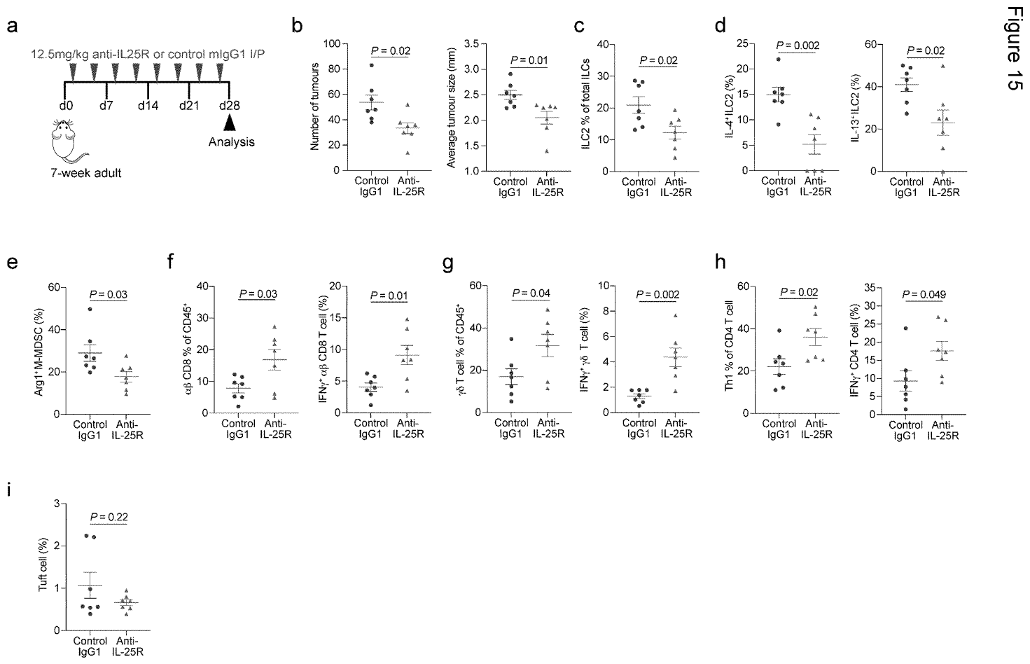

FIG. 16: Neutralisation of IL-25 with anti-IL-25 antibody inhibits the IL-25-ILC2-M-MDSC axis and reduces tumour burden a, Number of tumours and average tumour size in anti-IL-25 or control antibody-treated Apc^{1322T/+} mice (control, n=21; anti-IL-25, n=21). b,c, Frequency of tumour ILC2s (b) and G-MDSCs (c) in anti-IL-25 or control antibody-treated Apc^{1322T/+} mice (control, n=13; anti-IL-25, n=13). d, Frequency (left) and Arg1 expression (right) in tumour M-MDSCs in anti-IL-25 or control antibody-treated Apc^{1322T/+} mice (control, n=13; anti-IL-25, n=13). e, IFNγ expression in tumour αβ CD4 T cells and αβ CD8 T cells in anti-IL-25 or control antibody-treated Apc^{1322T/+} mice (control, n=13; anti-IL-25, n=13). Data collected from gender-matched mice at 11-weeks of age, treated with anti-IL-25 or control antibody for 8 weeks, starting from 3-weeks of age. Data pooled from two or more independent experiments; error bars show mean±SEM. Each dot indicates an individual mouse. Statistical significance determined by unpaired two-tailed t-test.

### EXAMPLES

### Summary of Examples 1 to 4

IL-25 and ILC2s help defend the host against intestinal parasitic helminth infection, and are associated with inappropriate allergic reactions. Recently, it was reported that IL-33-activated ILC2s augment protective tissue-specific cancer immunity. Here the inventors show a diametrically opposite role for IL-25-activated ILC2s in the intestine where they create a cancer-permissive microenvironment. Higher tumour IL25 gene expression is associated with reduced survival of colorectal cancer (CRC) patients, and IL-25R-expressing ILC2s are elevated in human CRC tumours. In a mouse model of spontaneous intestinal tumorigenesis, exogenous IL-25 increases tumours, whilst ablation of IL-25-signalling reduces tumour burden and virtually doubles life-expectancy, while reducing IL-25-responsive tumour ILC2s and shifting the balance towards cytotoxic CD8 T cell infiltration. Mice genetically lacking ILC2s are also more resistant to tumour development and showed prolonged survival, even when treated with exogenous IL-25. Like IL-25-deficient mice, ILC2-deficient mice had fewer tumour-resident immunosuppressive myeloid-derived suppressor cells (MDSCs), and showed improved anti-tumour innate and adaptive immunity. Thus, the roles of the innate epithelium-derived cytokines IL-25 and IL-33, and ILC2s in cancer cannot be generalised and may take pro-or anti-cancer phenotypes dependent on local microenvironmental cues within specific tissues or organs. Notably, IL-25-signalling blockade decreased ILC2s, MDSCs and tumorigenesis suggesting immunotherapeutic studies may be warranted in CRC.

### Example 1: IL-25 is Associated With Human and Mouse Colorectal Cancer

ILC2s are known to act as sentinels within barrier tissues, responding rapidly to acute immune perturbation by releasing effector cytokines. However, the tissue specific microenvironmental cues that differentially regulate innate ILC2-mediated immunity, in the context of a chronic cancer-permissive tissue niche, are poorly understood. Colorectal cancer (CRC) is the second most common cause of cancer-related death worldwide, and is expected to increase by a further 60% by 2030. Analysis of CRC tumour gene expression on large publicly available databases, stratified by IL25-high or IL25-low gene expression, identified significantly decreased overall, and disease-free, patient survival in the IL25-high group (FIG. 1a, b, FIG. 5a). Conversely, tumour IL33 gene expression, another potential ILC2-activating cytokine, did not associate with differential patient survival (FIG. 1c). Furthermore, human CRC tumours were enriched for IL-25 receptor IL-17BR (IL-25R) positive Lin-CD127+CRTH2+ILC2s, within the total ILC populations, as compared to normal colonic tissue (FIG. 1d, e), similar to recent reports. By contrast, CD8 T cells and Th1 cells (T-bet+CD4+T cells), normally associated with anti-tumour activity, did not increase or were reduced (FIG. 1f, FIG. 5b).

Approximately 80% of human CRC have mutations in the adenomatous polyposis coli (APC) tumour suppressor gene. Consequently, the inventors investigated IL-25 expression in the Apc^{1322T/+} model of spontaneous intestinal tumorigenesis that produces a mutant APC protein that retains one β-catenin binding degradation repeat—a scenario that closely mirrors human APC protein truncations. Analysis of IL25-tomato reporter mice (IL25^{tom/+}Apc^{1322T/+}) identified EpCAM⁺DCLK⁺ tuft cells as the main source of IL25-tomato in mouse tumours (FIG. 1g, FIG. 6a), though there was no difference in tuft cell frequency and epithelial IL25-tomato expression between tumours and adjacent normal gut (FIG. 6b). It has been reported that ILC2s participate in a reciprocal circuit with tuft cells in anti-parasitic immunity. Intestinal ILC2s express KLRG1 but not the IL-33-receptor (ST2), and similar to human CRC, tumours from Apc^{1322T/+} mice also harboured increased frequencies of ILC2s (FIG. 1h, FIG. 6c), that expressed higher levels of the IL-25R (FIG. 1i, j). Conversely, intestinal tumour ILC2s did not express ST2, confirmed using two different antibody clones (FIG. 1i, FIG. 6d). Similar to human samples, tumours from Apc^{1322T/+} mice harboured fewer CD8 than adjacent normal epithelium, and a trend to fewer Th1 cells (FIG. 1k, FIG. 6e). γδ T cells also decreased in tumours (FIG. 6f). Furthermore, tumour CD8 T cells and γδ T cells showed decreased granzyme, perforin and IFNγ expression (FIG. 6g, h), suggesting impaired anti-tumour cytotoxic functions. By contrast monocytic and granulocytic myeloid-derived suppressor cells (M-MDSCs and G-MDSCs), which can suppress anti-tumour T cell immunity and effector functions, were significantly increased in tumours compared to the adjacent normal gut in Apc^{1322T/+} mice (FIG. 6i, j).

Collectively, these results demonstrate that IL-25 expression is associated with both human and mouse intestinal cancer, along with an increase in IL-25R-expressing ILC2s and immunosuppressive MDSCs, but fewer anti-tumour-associated T cells.

### Example 2: IL-25 Promotes Intestinal Tumorigenesis

To investigate the role of IL-25 in intestinal tumorigenesis the inventors injected Apc^{1322T/+} mice with recombinant IL-25 (rIL-25) (FIG. 2a) and found it elicited a substantial increase in tumour numbers (FIG. 2b, FIG. 7a, b). This was not due to a direct effect on tumour cell proliferation, as there was no difference in the percentage of Ki67-expressing EpCAM⁺ tumour cells between rIL-25 and vehicle-treated mice (FIG. 7c). Instead, there was a dramatic increase in tumour infiltrating ILC2s (FIG. 2c), and a decrease in T-bet⁺ILC1s (FIG. 2d, FIG. 7d), Th1 and CD8 T cells (FIG. 2e), all known producers of IFNγ which decreases intestinal tumours. ILC2s also increased in the draining mesenteric lymph nodes (MLN), to a smaller degree (FIG. 7e). Tumour tuft cells increased in rIL-25-treated mice, suggesting positive feedback (FIG. 7f). There were no changes in other immune cell types analysed, including MDSCs, macrophages, cDCs, pDCs, mast cells, γδ T cells and Tregs (FIG. 2f, FIG. 7g-k). Notably, there was no change in Gata3⁺ CD4 Th2 cells indicating that IL-25 preferentially acts on innate ILC2s in the tumour microenvironment (FIG. 2g).

The inventors next generated IL-25-deficient (Il25^{tom/tom}) Apc^{1322T/+} mice and compared their tumorigenesis to the IL-25-replete Apc^{1322T/+} mice. Il25^{tom/tom} Apc^{1322T/+} mice developed considerably fewer and smaller tumours (FIG. 2h) and showed a striking increase in survival when compared to Apc^{1322T/+} mice, with a 90% and 81% increase in median survival in female and male mice, respectively (FIG. 2i, FIG. 8a). This was associated with a dramatic decrease in tumour and MLN ILC2s (FIG. 2j, FIG. 8b) and a concomitant increase in tumour ILC1s (FIG. 2k).

Although the inventors did not observe a change in tumour Th1 or CD8 T cell frequency (FIG. 2l), tumour M-MDSCs and G-MDSCs were substantially decreased in Il25^{tom/tom} Apc^{1322T/+} mice (FIG. 2m). Other immune cells were unchanged (FIG. 8c-h). The frequency of Th2 cells was also unaffected by IL-25-deficiency (FIG. 2n), suggesting again that IL-25 preferentially regulates type-2 innate, rather than adaptive, lymphocytes in tumours. Together, these data show that IL-25 promotes intestinal tumorigenesis by supporting ILC2s and suppressive MDSCs.

### Example 3: ILC2s Promote a Tumour-Permissive Microenvironment With Reduced Anti-Tumoural Immunity

To determine whether ILC2s could be responsible for IL-25-dependent intestinal tumorigenesis, the inventors generated ILC2-deficient (Rora^{f/f}Il7r^{Cre/+}) Apc^{1322T/+} mice (FIG. 9a), and revealed a substantial reduction in tumour numbers and size (FIG. 3a) and a notable increase in life-expectancy compared to Cre-control Apc^{1322T/+} mice (Rora^{+/+}Il7r^{Cre/+}) (FIG. 3b, FIG. 9b). In keeping with this improved survival, ILC2-deficiency led to increased tumour infiltrating ILC1s, Th1 and CD8 T cells (FIG. 3c, d), and restored γδ T cell anti-tumour effector functions, with increased granzyme, perforin and IFNγ expression (FIG. 3e, FIG. 9c), while Th2 cells were unchanged (FIG. 3f). This anti-tumour environment was likely further enhanced by the reduction of immune-suppressive M-MDSCs and G-MDSCs in tumours (FIG. 3g), which have been proposed to inhibit the anti-tumoural functions of innate γδ and adaptive T cells, while there was no change in other immune cell types including Tregs (FIG. 9d-h).

To confirm that the pro-tumoural effect of exogenous IL-25 is ILC2 dependent, the inventors injected ILC2-deficient Apc^{1322T/+} mice with rIL-25 using the same protocol as above.

Critically, there was no increase in tumour burden or tumour size in ILC2-deficient mice treated with rIL-25 (FIG. 3h, FIG. 10a). rIL-25 treatment did not induce ILC2-like cells in tumours from ILC2-deficient Apc^{1322T/+} mice (FIG. 10b), or affect the frequency of tumour infiltrating ILC1s (FIG. 3i). Interestingly, the inventors still observed a decrease in CD8 T cells and a similar trend in Th1 cells (FIG. 3j), suggesting that exogenous rIL-25 may act on CD8 T cells independently of ILC2s. However, in this scenario the reduction in CD8 T cells alone did not result in increased tumorigenesis in the absence of ILC2s, perhaps due to the fact that ILC2-deficient mice already have more elevated CD8 T cell infiltration (FIG. 3d). Similar to rIL-25 treatment in ILC2-replete mice, there was no difference in other immune cell types, including Th2 cells, with the exception of γδ T cells (FIG. 3k, FIG. 10c-i). The inventors again observed an increase in tuft cell frequency in tumours (FIG. 10j), which did not promote tumours in the absence of ILC2S.

ILC2s are defined by their expression of IL-13, and several studies have shown that IL-13, and more recently ILC2s, can promote MDSCs to suppress anti-tumour T cells in both mouse and human cancers. IL-13, and another ILC2 cytokine IL-4, signal through STAT6, and STAT6 deletion reduces MDSCs and tumours, while restoring anti-tumour CD8 immunity in Apc^{min/+} mice. The inventors found that M-MDSCs from Apc^{1322T/+} intestinal tumours express and upregulate the receptors IL-4RA and IL-13RA1 subunits as compared to the few M-MDSCs in the normal gut (FIG. 3l, m), whilst G-MDSCs expressed IL-4RA but not IL-13RA1 (FIG. 3n, FIG. 11a), indicating that MDSCs are capable of responding to IL-4 and IL-13 produced by ILC2s. The decrease in MHC-II expressing M-MDSCs in tumours is in line with a less differentiated and more suppressive phenotype (FIG. 11b). MDSCs are enriched in Apc^{1322T/+} intestinal tumours (FIG. 6j) and decreased in ILC2-deficient tumours (FIG. 3g). Taken together the data demonstrate that MDSCs act downstream of the IL-25:ILC2 axis.

### Example 4: Therapeutic Pathway Conservation in Human CRC

The results presented herein demonstrate that the innate IL-25:ILC2s axis supports intestinal tumour progression at least in part through promoting MDSCs and suppressing anti-tumoural T cells. To investigate whether these mechanisms are conserved in humans, the inventors analysed immune cells in paired unselected human CRC and normal colonic tissue samples. Th1 and CD8 T cell infiltration is amongst the strongest positive prognostic factors for survival of human CRC patients across all stages of disease. Notably, correlation analysis showed a significant negative correlation between tumour CD8 T cells and ILC2s in human CRC (FIG. 4a), and between Th1 cells and ILC2s (FIG. 4b), suggesting that CRCs with a higher ILC2 frequency may have impaired anti-tumour T cell responses. This is in line with the mouse data presented herein, where ILC2-deficient mice had improved CD8 and Th1 cell infiltration in tumours (FIG. 3d) and decreased tumour burden (FIG. 3a).

Furthermore, human CRCs had increased CD11b⁺CD14⁻CD15⁺CD33^{int} G-MDSCs (FIG. 4c, FIG. 12a), and CD11b⁺CD14⁺CD15⁻CD33^{high}HLA-DR^{low/-}M-MDSCs had decreased HLA-DR compared to the adjacent normal gut (FIG. 4d), similar to in Apc^{1322T/+} mice. Strikingly, tumour M-MDSCs positively correlated with ILC2 frequency in CRC patients (FIG. 4e). This association suggests that human M-MDSC recruitment in CRC mirrors the ILC2-dependent requirement for M-MDSC maintenance observed in mouse intestinal tumours. Notably, tumour infiltrating CD8 T cells negatively correlated with M-MDSCs in human CRC (FIG. 4f), consistent with the ability of M-MDSCs to suppress CD8 T cells.

The similar immune cell representation in human CRC and Apc^{1322T/+} mouse tumours suggest potential mechanistic conservation. The inventors assessed whether interrupting the innate IL-25:ILC2 immune axis could be targeted therapeutically, given the need for new drugs to treat CRC patients and the disappointing outcome of the recent phase III clinical trial IMblaze370 using the anti-PD-L1 checkpoint inhibitor atezolizumab to treat CRC patients (>98% had mismatch-repair-proficient CRC), which failed to meet the primary end point. The inventors confirmed expression of several checkpoint ligands on CRC MDSCs including CD86 (ligand for CTLA-4), PD-L1 (ligand for PD-1), HVEM (ligand for BTLA), CD155 and CD112 (ligand for TIGIT) on M-MDSCs, and PD-L1 and CD112 on G-MDSCs (FIG. 12b). CD8 T cells in CRC showed an exhausted phenotype with increased expression of checkpoint receptors PD-1, CTLA-4 and LAG-3 and decreased T-bet (FIG. 12c-e), which is associated with poor CD8 T cell effector function. This suggests that CD8 T cells in CRC have the potential to respond to checkpoint therapy. However, the limited efficacy of checkpoint inhibitors in cancer treatments has been linked to MDSCs, and the data presented herein raised the possibility that blocking the IL-25:ILC2 response would also ameliorate the “difficult to treat” immunosuppressive MDSCs.

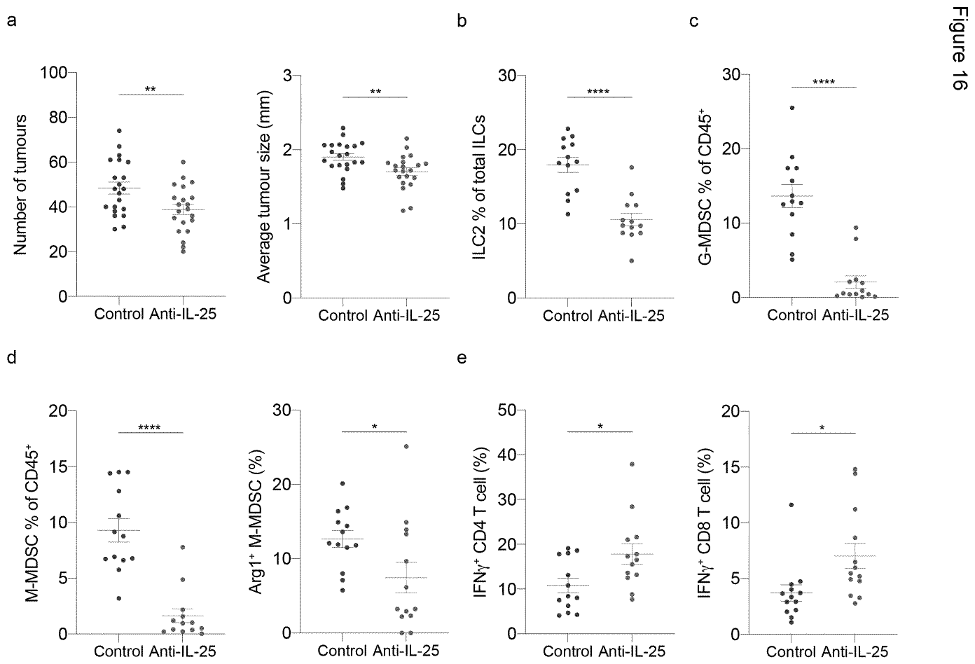

The inventors treated young-adult Apc^{1322T/+} mice with neutralising anti-IL25R antibody (clone D9.2) or control (FIG. 4g). Strikingly, anti-IL25R-treated mice developed considerably fewer and smaller tumours (FIG. 4h), had fewer tumour and MLN ILC2s (FIG. 4i, FIG. 13a), a reduction in tumour M-MDSCs and G-MDSCs (FIG. 4j), and shifting the balance to anti-tumour infiltrating ILC1s (FIG. 4k) and CD8 T cells (FIG. 4l). Tuft cells, the main source of intestinal IL-25, also decreased in anti-IL25R treated mice (FIG. 4m), in line with the decreased tumour ILC2s and interrupted ILC2-tuft cell circuit. Tumour-bearing mice and human cancer patients have a systemic increase in MDSCs, and anti-IL25R treatment in Apc^{1322T/+} mice also restored distant lung MDSCs to levels equivalent to wild type non-tumour bearing mice (FIG. 4n, FIG. 13b), suggesting systemic improvement and reflecting the reduced intestinal tumour burden. Again, blocking IL-25 signalling did not alter adaptive Th2 cell frequency (FIG. 40) or other cells analysed in tumours (FIG. 13c-i). Thus, therapeutic anti-IL-25R treatment shifts the intestinal immune response from a suppressive innate-immune tumour microenvironment to active anti-tumoural immunity.

Similar results were obtained when the inventors treated Apc^{1322T/+} mice with neutralising anti-IL25 antibody (RH2.5_R71V—with the variable domains expressed within the mouse IgG1 framework to enable in vivo studies in mice). Anti-IL-25 significantly reduced intestinal tumour numbers and size in the Apc-induced model of intestinal tumourigenesis as compared to isotype control (FIG. 16a). The anti-IL-25 mAb also markedly reduced the frequency of ILC2s (FIG. 16b) and this correlated with a striking reduction in myeloid-derived suppressor cells (MDSC) (FIG. 16c and d) which are known to inhibit anti-cancer responses. Indeed, by inhibiting IL-25 there was a concomitant increase in anti-cancer IFN-gamma-producing T cells (FIG. 16e) that correlated with the decrease in tumours. Thus, therapeutic anti-IL-25 treatment shifts the intestinal immune response from a suppressive innate-immune tumour microenvironment to active anti-tumoural immunity.

### Discussion—Examples 1 to 4

Our results demonstrate critical roles for IL-25 and ILC2s in maintaining the chronic cancer-permissive microenvironment that prevents the ascendency of anti-tumoural immunity in intestinal cancer. Notably, this immune-suppressive niche was unlikely to be dependent on adaptive Th2 cells, which did not change numerically in intestinal tumours in response to IL-25-deficiency, rIL-25 treatment, or ILC2-deficiency, but was dependent on ILC2s. A recent study reported that tissue-specific IL-33 can activate ILC2s to induce anti-tumour immunity in mouse models of orthotopic pancreatic cancer, but not in subcutaneous grafts of pancreatic cancer (Moral, J. A. et al. ILC2s amplify PD-1 blockade by activating tissue-specific cancer immunity. Nature 579, 130-135, doi: 10.1038/s41586-020-2015-4 (2020)). Strikingly, the data presented herein now demonstrate that tissue specialisation of IL-25-responsive intestinal ILC2s have diametrically opposite function, instead leading to them sustaining a tumorigenic niche in the intestine. Thus, the roles of the epithelium-derived cytokines IL-25 and IL-33, and ILC2s in tumorigenesis are likely to be location-dependent and organ specific, and not easily generalisable. Although it has been suggested that a small proportion of tuft cells may act as tumour stem cells, ILC2-deficiency decreased tumour burden but not tuft cell frequency, emphasising the critical role of ILC2s in this process. Notably, serum IL-25 is elevated in advanced CRC, and CRC is largely resistant to current immunotherapy. The results presented herein identify the IL-25-ILC2-MDSC axis as a novel immunotherapeutic target, and indicate that equivalent mechanisms may operate in human CRC. Since the anti-IL25R monoclonal antibody (clone D9.2) and the anti-IL25 monoclonal antibody (RH2.5_R71V) also inhibit human IL-25R signalling, this raises the possibility for future human immunotherapeutic studies in treating CRC. Furthermore, as anti-IL25R and anti-IL25 shift the balance away from MDSCs and towards anti-tumoural CD8 T cell infiltration, the beneficial effects may be potentiated by combined therapies with currently ineffectual checkpoint inhibitors.

### Methods—Examples 1 to 4

### Mice

All wild-type and Apc^{1322T/+} mice (Pollard, P. et al. The Apc 1322T mouse develops severe polyposis associated with submaximal nuclear beta-catenin expression. Gastroenterology 136, 2204-2213.e2201-473 2213, doi: 10.1053/j.gastro.2009.02.058 (2009)) in this study were on a C57BL/6 background and bred in-house. Mice were maintained at the Medical Research Council ARES animal facility under specific pathogen-free conditions. Breeders were placed on breeding diet (ENVIGO, 2919), while all experimental animals were on standard diet (SDS, 801722). All mice used in this study were females and were euthanised between 11 to 12 weeks of age, unless stated otherwise. For survival studies, mice were culled when showing clinical signs of pale feet, piloerection, hunching and weight loss, and the age recorded. No statistical tests were used to predetermine sample sizes. All animal experiments performed were under the approval of the UK Home Office.

### Generation of Il25-Tomato Gene-Targeted Mice

The Il25-knockout-tomato reporter mice were generated through recombineering (Liu, P., Jenkins, N. A. & Copeland, N. G. A highly efficient recombineering-based method for generating conditional knockout mutations. Genome Res 13, 476-484, doi: 10.1101/gr.749203 (2003); Warming, S., Costantino, N., Court, D. L., Jenkins, N. A. & Copeland, N. G. Simple and highly efficient BAC recombineering using galK selection. Nucleic Acids Res 33, e36, doi: 10.1093/nar/gni035 (2005)).

Neomycin-negative and Cre-recombinase-negative mice were backcrossed onto C57BL/6 background at least six times. Genotyping was through using PCR primers (forward, GACAGAATTGCAGATGCTATTACTACGACC; SEQ ID NO: 75) and (reverse, ACCTCCTCGCCCTTGCTCACCAT; SEQ ID NO: 76) for the targeted product (380bp), and (forward, GGCTAGGCTTCCAGGCTTCCAG; SEQ ID NO: 77) and (reverse, GGGGTTCTTGCTCTTTGCTGGG; SEQ ID NO: 78) for the wild-type product (200bp).

### Recombinant IL-25 Treatment and IL-17BR Blockade

Mice were injected intraperitoneally with 0.04 mg/kg of recombinant mouse IL-25 (Janssen) in PBS, three times per week for eight weeks, starting from weaning at three weeks of age. For IL-25R (IL-17BR) blockade, mice were injected intraperitoneally with 12.5 mg/kg of anti-IL25R (clone D9.2) 284 or control mIgG1 (LifeArc), twice a week for seven weeks, starting from three weeks of age. After the final injection, mice were euthanised and samples collected.

### Animal Procedures

Gender, background and age-matched mice, as described above, were euthanised and tissues harvested. Small intestines were flushed with cold PBS+10 mM HEPES (Gibco), opened longitudinally, and fixed in methacarn (60% methanol, 30% chloroform and 10% acetic acid glacial) for 24 hours and subsequently washed with PBS. Tumour numbers were counted, and tumour size measured with a ruler under a light microscope. For tumour scoring, mouse genotypes and/or treatment conditions were blind to the examiner whenever possible, except in occasional unavoidable cases where only one genotype was present at an age-matched timepoint. Spleens were collected and weighed immediately.

### Human Samples

All tumour samples were surgically resected primary CRCs performed at Cambridge University Hospitals NHS Trust and processed on the same day for analysis. All patients gave written consent to participate in this study. The study was approved by the Wales Research Ethics Committee 7 (15/WA/0131). No statistical tests were used to predetermine sample sizes.

### Cell Isolation

To obtain single cell suspensions of mouse tumours and adjacent normal gut, tumours and 2 cm pieces of adjacent normal gut were collected separately, followed by mechanical dissociation in RPMI-1640 containing 10 mM HEPES 309 (Gibco) and 2% foetal calf serum (FCS). Samples were digested with 62.5 μg/mL Liberase TL (Roche) and 0.125 KU/mL DNase I (Sigma) for 30 minutes at 37° C. whilst shaking.

Subsequently, cells were passed through a 50 μm filter and washed at least twice with PBS+2% FCS before antibody staining for flow cytometry. In experiments where adjacent normal epithelial fraction and lamina propria were analysed separately, tumours were removed and the remaining intestine was cut into 2 cm pieces and incubated with 1 mM dithiothreitol (Melford) and 5 mM EDTA in RPMI+2% FCS+10 mM HEPES for 2×20 minutes, shaking at 37° C., to collect the epithelial fraction.

Subsequently, samples were digested with 62.5 μg/mL Liberase TL (Roche) and 0.125 KU/mL DNase I (Sigma) in RPMI+2% FCS+10 mM HEPES at 37° C., shaking, for 45 minutes to collect lamina propria cells. After digestion, cells were passed through a 70 μm cell strainer and washed with PBS+2% FCS before antibody staining. Mesenteric lymph nodes were mechanically dissociated in RPMI, filtered through a 50 μm cell strainer, and washed with PBS+2% FCS before antibody staining. Fresh, surgically-resected human CRC and adjacent normal tissue were collected in sterile PBS and transported on ice before sample processing. Samples were mechanically dissociated into small pieces and digested with 100 μg/mL Liberase TM (Roche) and 0.2 KU/mL DNase I (Sigma) in RPMI, and incubated at 37° C. for 30 minutes. Subsequently, cells were filtered through a 70 μm cell strainer and washed with PBS+2% FCS before antibody staining for flow cytometry.

### Flow Cytometry

For surface staining, single cell suspensions were 333 blocked with anti-CD16/32 antibody (Fc block, clone 2.4G2) and Human TruStain FcX (Biolegend, 422302) for mouse and human samples in PBS+2% FCS respectively, and incubated with fluorochrome-conjugated or biotinylated antibodies on ice for 30 minutes in the dark. If necessary, samples were then incubated with fluorochrome-conjugated streptavidin on ice for a further 30 minutes.

For intracellular and nuclear staining of mouse samples, cells were fixed in 2% paraformaldehyde (ThermoFisher) for 45 minutes at room temperature in the dark, washed and subsequently stained with fluorochrome-conjugated antibodies for 45 minutes at room temperature in 1× Permeabilisation Buffer (eBioscience, 00-8333-56). For intracellular DCLK1 staining for tuft cell identification, fixed cells were incubated with primary anti-DCLK1 antibody (rabbit polyclonal, ab31704) for 45 minutes at room temperature, washed, and subsequently incubated with a secondary donkey-anti-rabbit AF647-conjugated antibody (ab150075) for another 45 minutes in 1× Permeabilisation Buffer. For intracellular and nuclear staining of human samples, cells were fixed using the True-Nuclear Transcription Factor Buffer Set (Biolegend, 424401) according to the manufacturer's protocol, and stained for intracellular and nuclear markers with fluorochrome-conjugated antibodies for 30 minutes, at room temperature and in the dark.

For analysis of IFNγ, perforin and granzyme expression by CD8 and γδ T cells, tumours and adjacent normal epithelium were processed into single cell suspensions, and stained with fluorochrome-conjugated antibodies as above. Subsequently, CD8 and γδ T cells were sorted using an iCyt Synergy cell sorter (Sony Biotechnology), and cultured with plate-coated anti-CD3e (Biolegend, 100360) and 10 ng/mL rm-IL-2 (Biolegend, 575406) for 16 hours. Protein Transport Inhibitor Cocktail (eBioscience, 00-4980-93) was added for the last 4 hours of culture, before antibody staining. Cells were blocked with anti-CD16/32 and stained for surface markers in PBS+2% FCS, on ice for 30 minutes and in the dark. Subsequently, cells were fixed using the Foxp3 Transcription Factor Staining Buffer Set (ebioscience, 00-5523-00) according to the manufacturer's protocol, and stained for intracellular markers with fluorochrome-conjugated antibodies for 30 minutes, at room temperature and in the dark.

Following intracellular staining, samples were washed serially with indicated 1× Permeabilisation Buffer and PBS+2% FCS, and resuspended in PBS+2% FCS for analyses through the BD LSRFortessa Flow Cytometer and FlowJo v10.2

### Antibodies

For mouse experiments, antibodies used for surface staining include NK1.1 (PK136, BUV395), CD45 (30-F11, BV510), IL7Rα (SB/199, biotin), streptavidin BV605, CD8 (53-6.7, BV785), CD11b (M1/70, FITC), KLRG1 (2F1, PerCP-eFluor 710), CD3 (145-2C11, PE-Cy7), CD11c (N418, PE-Cy7), CD19 (eBio1D3, PE-Cy7), Gr1 (RB6-8C5, PE-Cy7), FcεRI (MAR-1, PE-Cy7), TER119 (TER-119, PE-Cy7), CD4 (GK1.5, AF700), CD3 (17A2, eF450), CD11b (M1/70, eF450), CD11c (N418, eF450), CD19 (eBio1D3, eF450), F4/80 (BM8, eF450), Gr1 (RB6-8C5, eF450), FcεRI (MAR-1, eF450), TCRβ (H57-597, eF450), TER119 (TER-119, eF450), ST2 (RMST2-2, AF488), streptavidin PE, CD11b (M1/70, BUV395), EpCAM (G8.8, BV421), Ly6C (HK1.4, BV711), Ly6G (1A8-Ly6g, PerCP-eFluor 710), MHC-II (M5/114.15.2, PE),

TCRγδ (GL3, BV605), FcεRI (MAR-1, FITC), CD11c (N418, 383 PerCP-Cy5.5), CD317 (eBio927, APC), MHC-II (M5/114.15.2, BV510), CD19 (6D5, BV605), CD45 (30-F11, BV785), Gr1 (RB6-8C5, PerCP-Cy5.5), Siglec-F (E50-2440, PE), CD11C (N418, AF700), CD3 (17A2, BV510), CD8 (53-6.7, PE-Cy7), CD45 (30-F11, AF700), CD4 (GK1.5, PerCP-Cy5.5), TCRγδ (GL3, APC), IL17BR (D9.2, AF647), IL13RA1 (13MOKA, PE), IL4RA (mIL4R-M1, PE), ST2 (D.J8, FITC), Fixable Viability Dye eFluor 780 (eBioscience) and appropriate isotype controls. Lineage includes CD3, CD4, CD8, CD11b, CD11c, CD19, Gr1, FceRI, NK1.1, and TER119, and in some cases individual lineage markers were analysed in a separate channel as indicated.

Antibodies used for intracellular and nuclear staining include GATA3 (L50-823, BV711), FOXP3 (FJK-16s, PE-Cy7), Tbet (eBio4B10, eF660), Ki67 (SolA15, PE-Cy7), IFNγ (XMG1.2, BV785), Granzyme (NGZB, PE), Perforin (S16009B, APC) and appropriate isotype controls.

For human samples, antibodies used for surface staining include IL17BR (D9.2, biotin), streptavidin BV421, CD56 (HCD56, BV650), CD4 (OKT4, BV785), CD45 (HI30, AF488), CRTH2 (BM16, PE), CD127 (A019D5, PE-Cy7), CD3 (OKT3, APC), FcεRI (AER-37 (CRA-1), APC), CD11b (ICRF44, APC), CD11c (Bu15, APC), CD14 (HCD14, APC), CD19 (HIB19, APC), CD8 (RPA-T8, APC), CD123 (6H6, APC), CD14 (HCD14, Pacific Blue), CD86 (IT2.2, BV711), CD15 (W6D3, PerCP-Cy5.5), CD33 (WM53, PE), HLA-DR (L243, PE-Cy7), CD56 (HCD56, APC), CD45 (H130, AF700), PD-L1 (29E.2A3, BV650), CD33 (WM53, BV785), CD155 (SKII.4, FITC), CD112 (TX₃₁, PE), HVEM (122, PE-Cy7), CD8 (RPA-T8, BV510), CD3 (OKT3, Super Bright 645), PD1 (EH12.2H7, PerCP-Cy5.5), LAG3 (11C3C65, PE-Cy7), CD11b (ICRF44, FITC), Fixable Viability Dye eFluor 780 (eBioscience) and appropriate isotype controls. Lineage includes CD3, CD4, CD8, CD14, CD19,CD11b, CD11c, FcεRI, and CD123. Antibodies used for intracellular and nuclear staining include FOXP3 (206D, FITC), Tbet (eBio4B10, eF660), CTLA4 (L3D10, PE) and appropriate isotype controls.

### Human Survival Analysis

The publicly available TCGA Firehose Legacy Colorectal Adenocarcinoma dataset was analysed using the online open-access cBioPortal resource (https://www.cbioportal.org/)5,6. All patient samples (222) with Agilent microarray mRNA data were included in the analysis, and the mRNA expression z-scores were calculated relative to all samples. Using the query “IL25: EXP>0”, CRC patients were separated into two groups based on tumour IL25 expression above (IL25high) or below (IL25low) the mean IL25 expression. The two groups were compared for available survival data, including overall and disease-free survival. The same method was repeated for tumour IL33 expression, and survival between IL33high and IL33low groups compared. Relapse-free survival and tumour IL25 expression data by Marisa, L. et al. (Gene Expression Classification of Colon Cancer into Molecular 454 Subtypes: Characterization, Validation, and Prognostic Value. PLOS Medicine 10, 455 e1001453, doi: 10.1371/journal.pmed.1001453 (2013)) were analysed using the online open-access R2: Genomics Analysis and Visualization Platform (http://r2.amc.nl). All patient samples (566) were included in the analysis, and segregated into two groups based on tumour IL25 expression above (IL25high) and below (IL25low) the mean tumour IL25 expression of all samples, and relapse-free survival compared.

### Statistical Analysis

Statistical significance for human survival analyses was calculated automatically by the built-in system on cBioPortal and the R2 platform, for analysis of the TCGA Firehose Legacy (Cerami, E. et al. The cBio Cancer Genomics Portal: An Open Platform for Exploring Multidimensional Cancer Genomics Data. Cancer Discovery 2, 401-404, doi: 10.1158/2159-8290.Cd-12-0095 (2012); Gao, J. et al. Integrative analysis of complex cancer genomics and clinical profiles using the cBioPortal. Sci Signal 6, pl1, doi: 10.1126/scisignal.2004088 (2013)) and Marisa et al. 2013 CRC datasets respectively. All other statistical analyses in this study were performed using the GraphPad Prism 8 software.

### Example 5—TCGA Data of IL25 Expression Across Different CMS Subtypes

Publicly available TCGA data of CRC patients were separated into the four CMS subtypes, and analysed for IL25 expression (FIG. 14). y-axis shows IL25 expression.

1. A method of treating, or ameliorating cancer of the intestine in a subject in need thereof, comprising administering an inhibitor of the IL-25:IL-25R signaling pathway to the subject, wherein the inhibitor is or comprises an anti-IL-25 antibody, or functional fragment or derivative thereof.

2. The inhibitor for use according to method of claim 1, wherein the cancer is colorectal cancer.

3. The method of claim 1, wherein the cancer comprises a mutation in the adenomatous polyposis coli (APC) tumour suppressor gene.

4. The method of, wherein the cancer comprises a tumour that has an increased frequency of group 2 innate lymphoid cells (ILC2s) compared to the ILC2 frequency in adjacent normal intestine.

5-7. (canceled)

8. The method of claim 1, wherein the inhibitor comprises:
an antibody VH domain, or functional fragment or derivative thereof, comprising:
a VH CDR 1 comprising the amino acid sequence shown in any one of SEQ ID NOs: 1, 2, 12, 13, 14, 15, 16, 17, 18, or 19;
a VH CDR2 comprising the amino acid sequence shown in SEQ ID NO: 3; and
a VH CDR3 comprising the amino acid sequence shown in any one of SEQ ID NOs: 4, 20, or 21; and
an antibody a VL domain, or functional fragment or derivative thereof, comprising:
a VL CDR1 comprising the amino acid sequence shown in SEQ ID NO: 5 or SEQ ID NO: 6;
a VL CDR2 comprising the amino acid sequence shown in SEQ ID NO: 7; and
a VL CDR3 comprising the amino acid sequence shown in any one of SEQ ID NOs: 8, 9, 44, or 79.

9. The method of claim 1, wherein the inhibitor comprises:
an antibody VH domain, or functional fragment or derivative thereof, comprising:
a VH CDR1 comprising the amino acid sequence shown in SEQ ID NO: 12;
a VH CDR2 comprising the amino acid sequence shown in SEQ ID NO: 3; and
a VH CDR3 comprising the amino acid sequence shown in SEQ ID NO: 20; and
an antibody a VL domain, or functional fragment or derivative thereof, comprising:
a VL CDR1 comprising the amino acid sequence shown in SEQ ID NO: 5 or SEQ ID NO: 6;
a VL CDR2 comprising the amino acid sequence shown in SEQ ID NO: 7, and
a VL CDR3 comprising the amino acid sequence shown in SEQ ID NO: 8 or SEQ ID NO: 9.

10. The method of claim 1, wherein the inhibitor comprises:
an antibody VH domain, or functional fragment or derivative thereof, comprising:
a VH CDR1 comprising the amino acid sequence shown in SEQ ID NO: 12;
a VH CDR2 comprising the amino acid sequence shown in SEQ ID NO: 3; and
a VH CDR3 comprising the amino acid sequence shown in SEQ ID NO: 20; and
an antibody a VL domain, or functional fragment or derivative thereof, comprising:
a VL CDR1 comprising the amino acid sequence shown in SEQ ID NO: 6;
a VL CDR2 comprising the amino acid sequence shown in SEQ ID NO: 7, and
a VL CDR3 comprising the amino acid sequence shown in SEQ ID NO: 44 or SEQ ID NO: 79.

11. (canceled)

12. The method of claim 1, wherein the inhibitor comprises:
an antibody VH domain comprising the amino acid sequence shown in SEQ ID NO: 24, and
an antibody VL domain comprising the amino acid sequence shown in SEQ ID NO: 11 or SEQ ID NO: 41,
wherein the antibody VH domain and/or antibody VL domain comprises 1 to 10, from 1 to 5, from 1 to 3, one amino acid substitution, or no amino acid substitutions, compared to the recited sequence, in residues that are not within a CDR.

13. The method of claim 1, wherein the inhibitor comprises:
an antibody VH domain comprising the amino acid sequence shown in SEQ ID NO: 25, and
an antibody VL domain comprising the amino acid sequence shown in SEQ ID NO: 41,
wherein the antibody VH domain and/or antibody VL domain comprises from 1 to 10, from 1 to 5, from 1 to 3, one amino acid substitution, or no amino acid substitutions, compared to the recited sequence, in residues that are not within a CDR.

14. The method of claim 1, wherein the inhibitor comprises:
an antibody VH domain comprising the amino acid sequence shown in SEQ ID NO: 26, and
an antibody VL domain comprising the amino acid sequence shown in SEQ ID NO: 41.
wherein the antibody VH domain and/or antibody VL domain comprises from 1 to 10, from 1 to 5. from 1 to 3. one amino acid substitution, or no amino acid substitutions, compared to the recited sequence, in residues that are not within a CDR.

15. The method of claim 1, wherein the inhibitor comprises:
an antibody VH domain comprising the amino acid sequence shown in SEQ ID NO: 25, and
an antibody VL domain comprising the amino acid sequence shown in SEQ ID NO: 46, wherein the antibody VH domain and/or antibody VL domain comprises from 1 to 10, from 1 to 5. from 1 to 3. one amino acid substitution, or no amino acid substitutions, compared to the recited sequence, in residues that are not within a CDR

16. (canceled)

17. A method of treating or ameliorating cancer of the intestine in a subject in need thereof, comprising administering an inhibitor of the IL-25:IL-25R signalling pathway to the subject, wherein the inhibitor is or comprises an anti-IL-25R antibody, or functional fragment or derivative thereof.

18. The method of claim 17, wherein the inhibitor comprises:
an antibody VH domain, or functional fragment or derivative thereof, comprising:
a VH CDR 1 comprising the amino acid sequence shown in SEQ ID NO: 49;
a VH CDR2 comprising the amino acid sequence shown in any one of SEQ ID NOs: 50, 57, 58, 59, 60, 61, 62, 63, or 64; and
a VH CDR3 comprising the amino acid sequence shown in SEQ ID NO: 51; and
an antibody a VL domain, or functional fragment or derivative thereof, comprising:
a VL CDR1 comprising the amino acid sequence shown in SEQ ID NO: 52;
a VL CDR2 comprising the amino acid sequence shown in SEQ ID NO: 53; and
a VL CDR3 comprising the amino acid sequence shown in SEQ ID NO: 54 or SEQ ID NO: 65.

19. The method of claim 17, wherein the inhibitor comprises:
an antibody VH domain, or functional fragment or derivative thereof, comprising:
a VH CDR1 comprising the amino acid sequence shown in SEQ ID NO: 49;
a VH CDR2 comprising the amino acid sequence shown in SEQ ID NO: 50 or SEQ ID NO: 64; and
a VH CDR3 comprising the amino acid sequence shown in SEQ ID NO: 51; and
an antibody a VL domain, or functional fragment or derivative thereof, comprising:
a VL CDR1 comprising the amino acid sequence shown in SEQ ID NO: 52;
a VL CDR2 comprising the amino acid sequence shown in SEQ ID NO: 53; and
a VL CDR3 comprising the amino acid sequence shown in SEQ ID NO: 65.

20. The method of claim 17, wherein the inhibitor comprises:
an antibody VH domain comprising the amino acid sequence shown in any one of SEQ ID NOs: 66, 67, 68, 69, 70, 80, 81, 82, 83, 84, 85, or 86, and
an antibody VL domain comprising the amino acid sequence shown in any one of SEQ ID NOs: 56, 71, 72, 87, 88, 89, 90, or 91;
wherein the antibody VH domain and/or antibody VL domain comprises from 1 to 10, from 1 to 5, from 1 to 3, one amino acid substitution, or no amino acid substitutions, compared to the recited sequence, in residues that are not within a CDR.

21. The method of claim 17, wherein the inhibitor comprises:
an antibody VH domain comprising the amino acid sequence shown in any one of SEQ ID NOs: 55, 66, 67, 68, 69, or 70, and
an antibody VL domain comprising the amino acid sequence shown in any one of SEQ ID NOs: 56, 71, or 72;
wherein the antibody VH domain and/or antibody VL domain comprises from 1 to 10, from 1 to 5, from 1 to 3, one amino acid substitution, or no amino acid substitutions, compared to the recited sequence, in residues that are not within a CDR.

22. The method of claim 17, wherein the inhibitor comprises:
an antibody VH domain comprising the amino acid sequence shown in any one of SEQ ID NOs: 68, 69, or 70, and
an antibody VL domain comprising the amino acid sequence shown in SEQ ID NO: 72.

23-25. (canceled)

26. The method of claim 1, wherein the method further comprises the administration of an immune checkpoint inhibitor.

27. The method of claim 26, wherein the immune checkpoint inhibitor is an inhibitor of the PD-1: PD-L1 pathway.

28. The method of claim 17, wherein the cancer is colorectal cancer.

29. The method of claim 17, wherein the cancer comprises a mutation in the adenomatous polyposis coli (APC) tumour suppressor gene.

30. The method of claim 17, wherein the cancer comprises a tumour that has an increased frequency of group 2 innate lymphoid cells (ILC2s) compared to the ILC2 frequency in adjacent normal intestine.

31. The method of claim 17, wherein the method further comprises the administration of an immune checkpoint inhibitor

32. The method of claim 31, wherein the immune checkpoint inhibitor is an inhibitor of the PD-1: PD-L I pathway.


SEQUENCE LISTING (95 sequences)

SEQ ID NO: 1 - protein, 5 aa, Artificial Sequence
  2c3 VH CDR 1
DYTMN

SEQ ID NO: 2 - protein, 4 aa, Artificial Sequence
  2c3 VH CDR 1
YTMN

SEQ ID NO: 3 - protein, 17 aa, Artificial Sequence
  2c3 VH CDR 2
LINPYNGGTSYNQNFKG

SEQ ID NO: 4 - protein, 13 aa, Artificial Sequence
  2c3 VH CDR 3
EGYDGYLYFAMDY

SEQ ID NO: 5 - protein, 15 aa, Artificial Sequence
  2c3 VL CDR 1
SASQGISNYLNWYQQ

SEQ ID NO: 6 - protein, 11 aa, Artificial Sequence
  2c3 VL CDR 1
SASQGISNYLN

SEQ ID NO: 7 - protein, 7 aa, Artificial Sequence
  2c3 VL CDR 2
YTSSLHS

SEQ ID NO: 8 - protein, 9 aa, Artificial Sequence
  2c3 VL CDR 3
QQYSKLPYT

SEQ ID NO: 9 - protein, 10 aa, Artificial Sequence
  2c3 VL CDR 3
QQYSKLPYTF

SEQ ID NO: 10 - protein, 121 aa, Artificial Sequence
  2C3 VH
EVQLQQSGPELVKPGASMKISCKASGYSFTDYTMNWVKQSHGKNLEWIGLINPYNGGTSYNQNFKGKATLTVDKSSSTAYMELLSLTSEDSAVYYCAREGYDGYLYFAMDYWGQGTSVTVS

SEQ ID NO: 11 - protein, 107 aa, Artificial Sequence
  2C3 VL
DIQMTQTTSSLSASLGDRVTISCSASQGISNYLNWYQQKADGTVELLIYYTSSLHSGVPSRFSGSGSGTDYSLTISNLEPEDIATYYCQQYSKLPYTFGGGTKLEIK

SEQ ID NO: 12 - protein, 5 aa, Artificial Sequence
  humanised 2c3 VH CDR 1
GYTMN

SEQ ID NO: 13 - protein, 5 aa, Artificial Sequence
  humanised 2c3 VH CDR 1
AYTMN

SEQ ID NO: 14 - protein, 5 aa, Artificial Sequence
  humanised 2c3 VH CDR 1
SYTMN

SEQ ID NO: 15 - protein, 5 aa, Artificial Sequence
  humanised 2c3 VH CDR 1
VYTMN

SEQ ID NO: 16 - protein, 5 aa, Artificial Sequence
  humanised 2c3 VH CDR 1
NYTMN

SEQ ID NO: 17 - protein, 5 aa, Artificial Sequence
  humanised 2c3 VH CDR 1
KYTMN

SEQ ID NO: 18 - protein, 5 aa, Artificial Sequence
  humanised 2c3 VH CDR 1
YYTMN

SEQ ID NO: 19 - protein, 5 aa, Artificial Sequence
  humanised 2c3 VH CDR 1
MYTMN

SEQ ID NO: 20 - protein, 13 aa, Artificial Sequence
  humanised 2c3 VH CDR 3
EDYDGYLYFAMDY

SEQ ID NO: 21 - protein, 13 aa, Artificial Sequence
  humanised 2c3 VH CDR 3
ENYDGYLYFAMDY

SEQ ID NO: 22 - protein, 122 aa, Artificial Sequence
  2C3 VH
EVQLQQSGPELVKPGASMKISCKASGYSFTDYTMNWVKQSHGKNLEWIGLINPYNGGTSYNQNFKGKATLTVDKSSSTAYMELLSLTSEDSAVYYCAREGYDGYLYFAMDYWGQGTSVTVSS

SEQ ID NO: 23 - protein, 17 aa, Artificial Sequence
  2C3 VH leader sequence.
MVLSLLYLLTALPGILS

SEQ ID NO: 24 - protein, 122 aa, Artificial Sequence
  humanised 2C3 VH consensus
  SITE (30)..(30): Xaa is Ser or Thr
  SITE (31)..(31): Xaa is Gly, Asp, Ala, Ser, Val, Asn, Lys, Tyr or Met
  SITE (48)..(48): Xaa is Met or Ile
  SITE (72)..(72): Xaa is Val or Arg
  SITE (100)..(100): Xaa is Asn, Asn or Gly
EVQLVESGAEVKKPGASVKVSCKASGYSFXXYTMNWVRQAPGQRLEWXGLINPYNGGTSYNQNFKGRVTLTXDTSASTAYLELNSLRSEDTGVYYCAREXYDGYLYFAMDYWGQGTLVTVSS

SEQ ID NO: 25 - protein, 122 aa, Artificial Sequence
  humanised 2C3 VH
EVQLVESGAEVKKPGASVKVSCKASGYSFSGYTMNWVRQAPGQRLEWMGLINPYNGGTSYNQNFKGRVTLTVDTSASTAYLELNSLRSEDTGVYYCAREDYDGYLYFAMDYWGQGTLVTVSS

SEQ ID NO: 26 - protein, 120 aa, Artificial Sequence
  humanised 2C3 VH
EVQLVESGAEVKKPGASVKVSCKASGYSFTGYTMNWVRQAPGQRLEWMGLINPYNGGTSYNQNFKGRVTLTRDTSASTAYLELNSLREDTGVYYCAREDYDGYLYFAMDYWGQGTLVVSS

SEQ ID NO: 27 - protein, 122 aa, Artificial Sequence
  humanised 2C3 VH
EVQLVESGAEVKKPGASVKVSCKASGYSFSGYTMNWVRQAPGQRLEWMGLINPYNGGTSYNQNFKGRVTLTRDTSASTAYLELNSLRSEDTGVYYCAREDYDGYLYFAMDYWGQGTLVTVSS

SEQ ID NO: 28 - protein, 122 aa, Artificial Sequence
  humanised 2C3 VH
EVQLVESGAEVKKPGASVKVSCKASGYSFTGYTMNWVRQAPGQRLEWMGLINPYNGGTSYNQNFKGRVTLTVDTSASTAYLELNSLRSEDTGVYYCAREDYDGYLYFAMDYWGQGTLVTVSS

SEQ ID NO: 29 - protein, 122 aa, Artificial Sequence
  humanised 2C3 VH
EVQLVESGAEVKKPGASVKVSCKASGYSFSDYTMNWVRQAPGQRLEWMGLINPYNGGTSYNQNFKGRVTLTVDTSASTAYLELNSLRSEDTGVYYCAREDYDGYLYFAMDYWGQGTLVTVSS

SEQ ID NO: 30 - protein, 122 aa, Artificial Sequence
  humanised 2C3 VH
EVQLVESGAEVKKPGASVKVSCKASGYSFTDYTMNWVRQAPGQRLEWMGLINPYNGGTSYNQNFKGRVTLTRDTSASTAYLELNSLRSEDTGVYYCAREDYDGYLYFAMDYWGQGTLVTVSS

SEQ ID NO: 31 - protein, 122 aa, Artificial Sequence
  humanised 2C3 VH
EVQLVESGAEVKKPGASVKVSCKASGYSFTDYTMNWVRQAPGQRLEWMGLINPYNGGTSYNQNFKGRVTLTVDTSASTAYLELNSLRSEDTGVYYCAREDYDGYLYFAMDYWGQGTLVTVSS

SEQ ID NO: 32 - protein, 122 aa, Artificial Sequence
  humanised 2C3 VH
EVQLVESGAEVKKPGASVKVSCKASGYSFSGYTMNWVRQAPGQRLEWIGLINPYNGGTSYNQNFKGRVTLTVDTSASTAYLELNSLRSEDTGVYYCAREDYDGYLYFAMDYWGQGTLVTVSS

SEQ ID NO: 33 - protein, 122 aa, Artificial Sequence
  humanised 2C3 VH
EVQLVESGAEVKKPGASVKVSCKASGYSFTGYTMNWVRQAPGQRLEWIGLINPYNGGTSYNQNFKGRVTLTRDTSASTAYLELNSLRSEDTGVYYCAREDYDGYLYFAMDYWGQGTLVTVSS

SEQ ID NO: 34 - protein, 122 aa, Artificial Sequence
  humanised 2C3 VH
EVQLVESGAEVKKPGASVKVSCKASGYSFTGYTMNWVRQAPGQRLEWIGLINPYNGGTSYNQNFKGRVTLTVDTSASTAYLELNSLRSEDTGVYYCAREDYDGYLYFAMDYWGQGTLVTVSS

SEQ ID NO: 35 - protein, 122 aa, Artificial Sequence
  humanised 2C3 VH
EVQLVESGAEVKKPGASVKVSCKASGYSFSDYTMNWVRQAPGQRLEWIGLINPYNGGTSYNQNFKGRVTLTVDTSASTAYLELNSLRSEDTGVYYCAREDYDGYLYFAMDYWGQGTLVTVSS

SEQ ID NO: 36 - protein, 122 aa, Artificial Sequence
  humanised 2C3 VH
EVQLVESGAEVKKPGASVKVSCKASGYSFTDYTMNWVRQAPGQRLEWIGLINPYNGGTSYNQNFKGRVTLTRDTSASTAYLELNSLRSEDTGVYYCAREDYDGYLYFAMDYWGQGTLVTVSS

SEQ ID NO: 37 - protein, 122 aa, Artificial Sequence
  humanised 2C3 VH
EVQLVESGAEVKKPGASVKVSCKASGYSFTDYTMNWVRQAPGQRLEWIGLINPYNGGTSYNQNFKGRVTLTVDTSASTAYLELNSLRSEDTGVYYCAREDYDGYLYFAMDYWGQGTLVTVSS

SEQ ID NO: 38 - protein, 122 aa, Artificial Sequence
  humanised 2C3 VH, including leader sequence
EVRLVQSGAEVKKPGESLKISCKASGYSFTDYTMNWVRQMPGKGLEWIGLINPYNGGTSYNQNFKGQVTISVDKSINTAYLQWSSLKATDTAMYYCAREGYDGYLYFAMDYWGQGTLVIVSS

SEQ ID NO: 39 - protein, 19 aa, Artificial Sequence
  humanised 2C3 VH leader sequence
MGSTAILGLLLAVLQGVCA

SEQ ID NO: 40 - protein, 127 aa, Artificial Sequence
  humanised 2C3 VL, including leader sequence
MRVPAQLLGLLLLWLPDTRCDIQMTQSPSSLSASVGDRVTITCSASQGISNYLNWYQQKPGKVPKLLIYYTSSLHSGVPSRFSGSGSGTDFTLTISSLQPEDVATYYCQQYSKLPYTFGQGTKLEIK

SEQ ID NO: 41 - protein, 107 aa, Artificial Sequence
  humanised 2C3 VL
DIQMTQSPSSLSASVGDRVTITCSASQGISNYLNWYQQKPGKVPKLLIYYTSSLHSGVPSRFSGSGSGTDFTLTISSLQPEDVATYYCQQYSKLPYTFGQGTKLEIK

SEQ ID NO: 42 - protein, 20 aa, Artificial Sequence
  humanised 2C3 VL leader sequence
MRVPAQLLGLLLLWLPDTRC

SEQ ID NO: 43 - protein, 22 aa, Artificial Sequence
  Human VL leader sequence
MDMRVPAQLLGLLLLWLPDTRC

SEQ ID NO: 44 - protein, 9 aa, Artificial Sequence
  M6 VL CDR 3
QQYLAFPYT

SEQ ID NO: 45 - protein, 452 aa, Artificial Sequence
  M6 VH
EVQLVESGAEVKKPGASVKVSCKASGYSFSGYTMNWVRQAPGQRLEWMGLINPYNGGTSYNQNFKGRVTLTVDTSASTAYLELNSLRSEDTGVYYCAREDYDGYLYFAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 107 aa, Artificial Sequence
  M6 VL
DIQMTQSPSSLSASVGDRVTITCSASQGISNYLNWYQQKPGKVPKLLIYYTSSLHSGVPSRFSGSGSGTDFTLTISSLQPEDVATYYCQQYLAFPYTFGQGTKLEIK

SEQ ID NO: 47 - protein, 214 aa, Artificial Sequence
  M6 Light Chain
DIQMTQSPSSLSASVGDRVTITCSASQGISNYLNWYQQKPGKVPKLLIYYTSSLHSGVPSRFSGSGSGTDFTLTISSLQPEDVATYYCQQYLAFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 146 aa, Homo sapiens
MYSHWPSCCPSKGQDTSEELLRWSTVPVPPLEPARPNRHPESCRASEDGPLNSRAISPWRYELDRDLNRLPQDLYHARCLCPHCVSLQTGSHMDPRGNSELLYHNQTVFYRRPCHGEKGTHKGYCLERRLYRVSLACVCVRPRVMG

SEQ ID NO: 49 - protein, 5 aa, Artificial Sequence
  D9.2 VH CDR 1
SYWMN

SEQ ID NO: 50 - protein, 17 aa, Artificial Sequence
  D9.2 VH CDR 2
RIDPYDSEIQYNQKFKD

SEQ ID NO: 51 - protein, 9 aa, Artificial Sequence
  D9.2 VH CDR 3
SGGFDWFAY

SEQ ID NO: 52 - protein, 11 aa, Artificial Sequence
  D9.2 VL CDR 1
RASENINSNLA

SEQ ID NO: 53 - protein, 7 aa, Artificial Sequence
  D9.2 VL CDR 2
DVTNLAD

SEQ ID NO: 54 - protein, 9 aa, Artificial Sequence
  D9.2 VL CDR 3
QHFWRPPYT

SEQ ID NO: 55 - protein, 129 aa, Artificial Sequence
  D9.2 VH
FFLATPTCVHSQVQLQQPGAELVRPGASVKLSCKTSGYTFISYWMNWVKQGGPEQGLEWIGRIDPYDSEIQYNQKFKDKAILTVDKSSSAAYMQLISLTSEDSAVYYCARSGGFDWFAYWGQGTLVTVS

SEQ ID NO: 56 - protein, 127 aa, Artificial Sequence
  D9.2 VL
MSVLTQVLALLLLWLTDARCDIQMTQSPASLSVSVGETVTITCRASENINSNLAWYQQKKGKSPQLLVYDVTNLADGVPSRFSGSGSGTQYSLKINSLQSEDFGSYYCQHFWRPPYTFGGGTNLEIK

SEQ ID NO: 57 - protein, 17 aa, Artificial Sequence
  Humanised D9.2 VH CDR 2
  SITE (12)..(12): Xaa is Asn or Ala
  SITE (16)..(16): Xaa is Lys or Gln
  SITE (17)..(17): Xaa is Asp or Gly
RIDPYDSEIQYXQKFXX

SEQ ID NO: 58 - protein, 17 aa, Artificial Sequence
  humanised D9.2 VH CDR2
RIDPYDSEIQYNQKFKG

SEQ ID NO: 59 - protein, 17 aa, Artificial Sequence
  humanised D9.2 VH CDR2
RIDPYDSEIQYNQKFQD

SEQ ID NO: 60 - protein, 17 aa, Artificial Sequence
  humanised D9.2 VH CDR2
RIDPYDSEIQYNQKFQG

SEQ ID NO: 61 - protein, 17 aa, Artificial Sequence
  humanised D9.2 VH CDR2
RIDPYDSEIQYAQKFKD

SEQ ID NO: 62 - protein, 17 aa, Artificial Sequence
  humanised D9.2 VH CDR2
RIDPYDSEIQYAQKFKG

SEQ ID NO: 63 - protein, 17 aa, Artificial Sequence
  humanised D9.2 VH CDR2
RIDPYDSEIQYAQKFQD

SEQ ID NO: 64 - protein, 17 aa, Artificial Sequence
  humanised D9.2 VH CDR2
RIDPYDSEIQYAQKFQG

SEQ ID NO: 65 - protein, 9 aa, Artificial Sequence
  humanised D9.2 VL CDR3
QHFWGPPYT

SEQ ID NO: 66 - protein, 118 aa, Artificial Sequence
  humanised D9.2 VH
EVQLVQSGAEVKKPGASVKVSCKTSGYTFISYWMNWVRQAPGQGLEWMGRIDPYDSEIQYNQKFKDRVTLTRDTSISTAYMELSRLRSDDTAVYYCATSGGFDWFAYWGQGTLVTVSS

SEQ ID NO: 67 - protein, 118 aa, Artificial Sequence
  humanised D9.2 VH B
  SITE (1)..(1): Xaa is Gln or Glu
  SITE (24)..(24): Xaa is Ala or Thr
  SITE (61)..(61): Xaa is Ala or Asn
  SITE (65)..(65): Xaa is Gln or Lys
  SITE (66)..(66): Xaa is Gly or Asp
  SITE (70)..(70): Xaa is Met or Leu
XVQLVQSGAEVKKPGASVKVSCKXSGYTFISYWMNWVRQAPGQGLEWMGRIDPYDSEIQYXQKFXXRVTXTRDTSISTAYMELSRLRSDDTAVYYCARSGGFDWFAYWGQGTLVTVSS

SEQ ID NO: 68 - protein, 118 aa, Artificial Sequence
  humanised D9.2 VH C (D9 HJ)
QVQLVQSGAEVKKPGASVKVSCKASGYTFISYWMNWVRQAPGQGLEWMGRIDPYDSEIQYNQKFKDRVTMTRDTSISTAYMELSRLRSDDTAVYYCARSGGFDWFAYWGQGTLVTVSS

SEQ ID NO: 69 - protein, 118 aa, Artificial Sequence
  humanised D9.2 VH D (D9 HH)
EVQLVQSGAEVKKPGASVKVSCKTSGYTFISYWMNWVRQAPGQGLEWMGRIDPYDSEIQYNQKFKDRVTLTRDTSISTAYMELSRLRSDDTAVYYCARSGGFDWFAYWGQGTLVTVSS

SEQ ID NO: 70 - protein, 118 aa, Artificial Sequence
  humanised D9.2 VH E (D9 HI)
EVQLVQSGAEVKKPGASVKVSCKTSGYTFISYWMNWVRQAPGQGLEWMGRIDPYDSEIQYAQKFQGRVTLTRDTSISTAYMELSRLRSDDTAVYYCARSGGFDWFAYWGQGTLVTVSS

SEQ ID NO: 71 - protein, 107 aa, Artificial Sequence
  humanised D9.2 VL
DIQMTQSPSSLSASVGDRVTITCRASENINSNLAWYQQKPGKAPKLLLYDVTNLADGVPSRFSGSGSGTDYTLTISSLQPEDFATYYCQHFWRPPYTFGGGTKVEIK

SEQ ID NO: 72 - protein, 107 aa, Artificial Sequence
  humanised D9.2 VL (D9 KE)
DIQMTQSPSSLSASVGDRVTITCRASENINSNLAWYQQKPGKAPKLLLYDVTNLADGVPSRFSGSGSGTDYTLTISSLQPEDFATYYCQHFWGPPYTFGGGTKVEIK

SEQ ID NO: 73 - protein, 327 aa, Artificial Sequence
  hinge stabilised IgG4 constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 74 - protein, 445 aa, Artificial Sequence
  heavy chain of the humanised D9.2
QVQLVQSGAEVKKPGASVKVSCKASGYTFISYWMNWVRQAPGQGLEWMGRIDPYDSEIQYNQKFKDRVTMTRDTSISTAYMELSRLRSDDTAVYYCARSGGFDWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 75 - DNA, 30 bp, Artificial Sequence
  Primer
gacagaattgcagatgctattactacgacc

SEQ ID NO: 76 - DNA, 23 bp, Artificial Sequence
  Primer
acctcctcgcccttgctcaccat

SEQ ID NO: 77 - DNA, 22 bp, Artificial Sequence
  Primer
ggctaggcttccaggcttccag

SEQ ID NO: 78 - DNA, 22 bp, Artificial Sequence
  Primer
ggggttcttgctctttgctggg

SEQ ID NO: 79 - protein, 10 aa, Artificial Sequence
  M6 VL CDR 3
QQYLAFPYTF

SEQ ID NO: 80 - protein, 118 aa, Artificial Sequence
  D9.2 VH Alt
QVQLQQPGAELVRPGASVKLSCKTSGYTFISYWMNWVKQGPEQGLEWIGRIDPYDSEIQYNQKFKDKAILTVDKSSSAAYMQLISLTSEDSAVYYCARSGGFDWFAYWGQGTLVTVSA

SEQ ID NO: 81 - protein, 118 aa, Artificial Sequence
  D9.2 VH HB
EVQLVQSGAEVKKPGASVKVSCKTSGYTFISYWMNWVRQAPGQGLEWIGRIDPYDSEIQYNQKFKDKAILTVDTSISTAYMELSRLRSDDTAVYYCARSGGFDWFAYWGQGTLVTVSS

SEQ ID NO: 82 - protein, 118 aa, Artificial Sequence
  D9.2 VH HC
EVQLVQSGAEVKKPGASVKVSCKTSGYTFISYWMNWVRQAPGQGLEWIGRIDPYDSEIQYNQKFKDRVTLTRDTSISTAYMELSRLRSDDTAVYYCATSGGFDWFAYWGQGTLVTVSS

SEQ ID NO: 83 - protein, 118 aa, Artificial Sequence
  D9.2 VH HD
EVQLVQSGAEVKKPGASVKVSCKTSGYTFISYWMNWVRQAPGQGLEWMGRIDPYDSEIQYNQKFKDKVTLTRDTSISTAYMELSRLRSDDTAVYYCATSGGFDWFAYWGQGTLVTVSS

SEQ ID NO: 84 - protein, 118 aa, Artificial Sequence
  D9.2 VH HE
EVQLVQSGAEVKKPGASVKVSCKTSGYTFISYWMNWVRQAPGQGLEWMGRIDPYDSEIQYNQKFKDRATLTRDTSISTAYMELSRLRSDDTAVYYCATSGGFDWFAYWGQGTLVTVSS

SEQ ID NO: 85 - protein, 118 aa, Artificial Sequence
  D9.2 VH HF
EVQLVQSGAEVKKPGASVKVSCKTSGYTFISYWMNWVRQAPGQGLEWMGRIDPYDSEIQYNQKFKDRVILTRDTSISTAYMELSRLRSDDTAVYYCATSGGFDWFAYWGQGTLVTVSS

SEQ ID NO: 86 - protein, 118 aa, Artificial Sequence
  D9.2 VH HG
EVQLVQSGAEVKKPGASVKVSCKTSGYTFISYWMNWVRQAPGQGLEWMGRIDPYDSEIQYNQKFKDRVTLTVDTSISTAYMELSRLRSDDTAVYYCATSGGFDWFAYWGQGTLVTVSS

SEQ ID NO: 87 - protein, 107 aa, Artificial Sequence
  D9.2 VK Alt
DIQMTQSPASLSVSVGETVTITCRASENINSNLAWYQQKKGKSPQLLVYDVTNLADGVPSRFSGSGSGTQYSLKINSLQSEDFGSYYCQHFWRPPYTFGGGTNLEIK

SEQ ID NO: 88 - protein, 107 aa, Artificial Sequence
  D9.2 VL KB
DIQMTQSPSSLSASVGDRVTITCRASENINSNLAWYQQKPGKAPKLLVYDVTNLADGVPSRFSGSGSGTQYTLTISSLQPEDFATYYCQHFWRPPYTFGGGTKVEIK

SEQ ID NO: 89 - protein, 107 aa, Artificial Sequence
  D9.2 VL KC
DIQMTQSPSSLSASVGDRVTITCRASENINSNLAWYQQKPGKAPKLLVYDVTNLADGVPSRFSGSGSGTDYTLTISSLQPEDFATYYCQHFWRPPYTFGGGTKVEIK

SEQ ID NO: 90 - protein, 107 aa, Artificial Sequence
  D9.2 VL KD
DIQMTQSPSSLSASVGDRVTITCRASENINSNLAWYQQKPGKAPKLLLYDVTNLADGVPSRFSGSGSGTQYTLTISSLQPEDFATYYCQHFWRPPYTFGGGTKVEIK

SEQ ID NO: 91 - protein, 107 aa, Artificial Sequence
  D9.2 VL KM
DIQMTQSPSSLSASVGDRVTITCRTSENIYSNLAWYQQKPGKAPKLLLYDATNLGDGVPSRFSGSGSGTDYTLTISSLQPEDFATYYCQHFWGPPYTFGGGTKVEIK

SEQ ID NO: 92 - protein, 502 aa, Homo sapiens
MSLVLLSLAALCRSAVPREPTVQCGSETGPSPEWMLQHDLIPGDLRDLRVEPVTTSVATGDYSILMNVSWVLRADASIRLLKATKICVTGKSNFQSYSCVRCNYTEAFQTQTRPSGGKWTFSYIGFPVELNTVYFIGAHNIPNANMNEDGPSMSVNFTSPGCLDHIMKYKKKCVKAGSLWDPNITACKKNEETVEVNFTTTPLGNRYMALIQHSTIIGFSQVFEPHQKKQTRASVVIPVTGDSEGATVQLTPYFPTCGSDCIRHKGTVVLCPQTGVPFPLDNNKSKPGGWLPLLLLSLLVATWVLVAGIYLMWRHERIKKTSFSTTTLLPPIKVLVVYPSEICFHHTICYFTEFLQNHCRSEVILEKWQKKKIAEMGPVQWLATQKKAADKVVFLLSNDVNSVCDGTCGKSEGSPSENSQDLFPLAFNLFCSDLRSQIHLHKYVVVYFREIDTKDDYNALSVCPKYHLMKDATAFCAELLHVKQQVSAGKRSQACHDGCCSL

SEQ ID NO: 93 - protein, 288 aa, Homo sapiens
MSLVLLSLAALCRSAVPREPTVQCGSETGPSPEWMLQHDLIPGDLRDLRVEPVTTSVATGDYSILMNVSWVLRADASIRLLKATKICVTGKSNFQSYSCVRCNYTEAFQTQTRPSGGKWTFSYIGFPVELNTVYFIGAHNIPNANMNEDGPSMSVNFTSPGCLDHIMKYKKKCVKAGSLWDPNITACKKNEETVEVNFTTTPLGNRYMALIQHSTIIGFSQVFEPHQKKQTRASVVIPVTGDSEGATVQVKFSELLWGGKGHRRLFHHSLLLRMSSLLSNALLPADTS

SEQ ID NO: 94 - protein, 120 aa, Artificial Sequence
  EF178110
EVQLVESGAEVKKPGASVKVSCKTSGYTFIDYYIHWVRQAPGQGLEWMGWINPKSGGTNYARNLRDRVTLTRDTSINTAYMELSRLRSDDTALYYCATLYGDYRPIQDSWGQGTLVTVSS

SEQ ID NO: 95 - protein, 107 aa, Artificial Sequence
  Y14869
DIQMTQSPSSLSASVGDRVTITCRASQGISNSLAWYQQKPGKAPKLLLYAASRLESGVPSRFSGSGSGTDYTLTISSLQPEDFATYYCQQYYSTPLTFGGGTKVEIK